SEQUENCE LISTING (201 sequences)

SEQ ID NO: 1 - protein, 448 aa, synthetic construct
  REGION 1..448: note = FS28-256 heavy chain (without LALA)
  VARIANT 50: note = Xaa can be any naturally occurring amino acid
  VARIANT 52: note = Xaa can be any naturally occurring amino acid
  REGION 50..52: note = MISC_FEATURE - Cannot be a glycosylation site
EVQLLESGGGLVQPGGSLRLSCAASGFTFTETYMSWVRQAPGKGLEWVSXIXPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 448 aa, synthetic construct
  REGION 1..448: note = FS28-256 heavy chain (with LALA)
  VARIANT 50: note = Xaa can be any naturally occurring amino acid
  VARIANT 52: note = Xaa can be any naturally occurring amino acid
  REGION 50..52: note = Cannot be a glycosylation site
EVQLLESGGGLVQPGGSLRLSCAASGFTFTETYMSWVRQAPGKGLEWVSXIXPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 215 aa, synthetic construct
  REGION 1..215: note = FS28-256-001, FS28-256-021 and FS28-256-023 light chain
  VARIANT 97: note = Xaa can be any naturally occurring amino acid
  VARIANT 98: note = Xaa can be any naturally occurring amino acid
  REGION 97..98: note = Cannot be a deamidation site
EIVLTQSPGTLSLSPGERATLSCRASQSVSSSYLAWYQQKPGQAPRLLIYGASSRATGIPDRFSGSGSGTDFTLTISRLEPEDFAVYYCQQHNQYPXXFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 445 aa, synthetic construct
  REGION 1..445: note = heavy chain of FS28-024 mAb without LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 445 aa, synthetic construct
  REGION 1..445: note = heavy chain of FS28-024 mAb with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - DNA, 1335 bp, synthetic construct
  misc_feature 1..1335: note = heavy chain of FS28-024 mAb without LALA
  source 1..1335: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgacgttcgactactggggccagggaaccttggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 7 - DNA, 1335 bp, synthetic construct
  misc_feature 1..1335: note = heavy chain of FS28-024 mAb with LALA
  source 1..1335: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgacgttcgactactggggccagggaaccttggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 8 - protein, 116 aa, synthetic construct
  REGION 1..116: note = FS28-024 mAb variable domain
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSS

SEQ ID NO: 9 - DNA, 348 bp, synthetic construct
  misc_feature 1..348: note = FS28-024 mAb variable domain
  source 1..348: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgacgttcgactactggggccagggaaccttggtcaccgtctcgagt

SEQ ID NO: 10 - protein, 8 aa, synthetic construct
  REGION 1..8: note = FS28-024 mAb CDR1 (IMGT)
GFTLSYSS

SEQ ID NO: 11 - protein, 8 aa, synthetic construct
  REGION 1..8: note = FS28-024 mAb CDR2 (IMGT)
ITPSTGYT

SEQ ID NO: 12 - protein, 9 aa, synthetic construct
  REGION 1..9: note = FS28-024 mAb CDR3 (IMGT)
ARRALTFDY

SEQ ID NO: 13 - protein, 5 aa, synthetic construct
  REGION 1..5: note = FS28-024 mAb CDR1 (Kabat)
YSSMS

SEQ ID NO: 14 - protein, 17 aa, synthetic construct
  REGION 1..17: note = FS28-024 mAb CDR2 (Kabat)
FITPSTGYTHYADSVKG

SEQ ID NO: 15 - protein, 7 aa, synthetic construct
  REGION 1..7: note = FS28-024 mAb CDR3 (Kabat)
RALTFDY

SEQ ID NO: 16 - protein, 215 aa, synthetic construct
  REGION 1..215: note = light chain of FS28-024 mAb
EIVLTQSPGTLSLSPGERATLSCRASQSVSSSYLAWYQQKPGQAPRLLIYGASSRATGIPDRFSGSGSGTDFTLTISRLEPEDFAVYYCQQASSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 645 bp, synthetic construct
  misc_feature 1..645: note = light chain of FS28-024 mAb
  source 1..645: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaagcttcttcttatcctctcacgttcggccaagggaccaaggtggaaatcaaacgtactgtggccgctcctagcgtgttcatttttccgccatccgacgagcagctcaagtccggcaccgcctccgtggtctgcctgctcaacaacttctaccctcgcgaagctaaggtccagtggaaggtcgacaatgccctgcagtccggaaactcgcaggaaagcgtgactgaacaggactccaaggactccacctattcactgtcctcgactctgaccctgagcaaggcggattacgaaaagcacaaagtgtacgcatgcgaagtgacccaccagggtctttcgtcccccgtgaccaagagcttcaacagaggagagtgt

SEQ ID NO: 18 - protein, 108 aa, synthetic construct
  REGION 1..108: note = variable domain of FS28-024 mAb
EIVLTQSPGTLSLSPGERATLSCRASQSVSSSYLAWYQQKPGQAPRLLIYGASSRATGIPDRFSGSGSGTDFTLTISRLEPEDFAVYYCQQASSYPLTFGQGTKVEIK

SEQ ID NO: 19 - DNA, 324 bp, synthetic construct
  misc_feature 1..324: note = variable domain of FS28-024 mAb
  source 1..324: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaagcttcttcttatcctctcacgttcggccaagggaccaaggtggaaatcaaa

SEQ ID NO: 20 - protein, 7 aa, synthetic construct
  REGION 1..7: note = FS28-024 mAb (IMGT) CDR1
QSVSSSY

SEQ ID NO: 21 - skipped

SEQ ID NO: 22 - protein, 9 aa, synthetic construct
  REGION 1..9: note = FS28-024 mAb (IMGT) CDR3
QQASSYPLT

SEQ ID NO: 23 - protein, 12 aa, synthetic construct
  REGION 1..12: note = FS28-024 mAb (Kabat) CDR1
RASQSVSSSYLA

SEQ ID NO: 24 - protein, 7 aa, synthetic construct
  REGION 1..7: note = FS28-024 mAb (Kabat) CDR2
GASSRAT

SEQ ID NO: 25 - protein, 9 aa, synthetic construct
  REGION 1..9: note = FS28-256 mAb CDR3 (IMGT)
QQSYYYPIT

SEQ ID NO: 26 - protein, 445 aa, synthetic construct
  REGION 1..445: note = FS28-024-051 mAb heavy chain without LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - DNA, 1335 bp, synthetic construct
  misc_feature 1..1335: note = FS28-024-051 mAb heavy chain without LALA
  source 1..1335: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgattttcgactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 28 - protein, 445 aa, synthetic construct
  REGION 1..445: note = FS28-024-051 mAb heavy chain with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - DNA, 1335 bp, synthetic construct
  misc_feature 1..1335: note = FS28-024-051 mAb heavy chain with LALA
  source 1..1335: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgattttcgactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 30 - protein, 116 aa, synthetic construct
  REGION 1..116: note = FS28-024-051 mAb variable domain
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSS

SEQ ID NO: 31 - DNA, 348 bp, synthetic construct
  misc_feature 1..348: note = FS28-024-051 mAb variable domain
  source 1..348: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgattttcgactactggggccagggaaccctggtcaccgtctcgagt

SEQ ID NO: 32 - protein, 9 aa, synthetic construct
  REGION 1..9: note = FS28-024-051 mAb CDR3 (IMGT)
ARRALIFDY

SEQ ID NO: 33 - protein, 7 aa, synthetic construct
  REGION 1..7: note = FS28-024-051 mAb CDR3 (Kabat)
RALIFDY

SEQ ID NO: 34 - protein, 9 aa, synthetic construct
  REGION 1..9: note = FS28-256-001 mAb CDR3 (IMGT) and (Kabat)
QQHNQYPNT

SEQ ID NO: 35 - protein, 445 aa, synthetic construct
  REGION 1..445: note = FS28-024-052 mAb heavy chain without LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - DNA, 1335 bp, synthetic construct
  misc_feature 1..1335: note = FS28-024-052 mAb heavy chain without LALA
  source 1..1335: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgcttttcgactactggggccagggaaccctggtcaccgtctcgtcggctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 37 - protein, 445 aa, synthetic construct
  REGION 1..445: note = FS28-024-052 mAb heavy chain with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - DNA, 1335 bp, synthetic construct
  misc_feature 1..1335: note = FS28-024-052 mAb heavy chain with LALA
  source 1..1335: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgcttttcgactactggggccagggaaccctggtcaccgtctcgtcggctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 39 - protein, 116 aa, synthetic construct
  REGION 1..116: note = FS28-024-052 mAb variable domain
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSS

SEQ ID NO: 40 - DNA, 348 bp, synthetic construct
  misc_feature 1..348: note = FS28-024-052 mAb variable domain
  source 1..348: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctgcttttcgactactggggccagggaaccctggtcaccgtctcgtcg

SEQ ID NO: 41 - protein, 9 aa, synthetic construct
  REGION 1..9: note = FS28-024-052 mAb CDR3 (IMGT)
ARRALLFDY

SEQ ID NO: 42 - protein, 7 aa, synthetic construct
  REGION 1..7: note = FS28-024-052 mAb CDR3 (Kabat)
RALLFDY

SEQ ID NO: 43 - protein, 9 aa, synthetic construct
  REGION 1..9: note = FS28-256-005 mAb CDR3 (IMGT) and (Kabat)
QQALGYPHT

SEQ ID NO: 44 - protein, 9 aa, synthetic construct
  REGION 1..9: note = FS28-256-027 mAb CDR3 (IMGT) and (Kabat)
QQTVPYPYT

SEQ ID NO: 45 - protein, 445 aa, synthetic construct
  REGION 1..445: note = FS28-024-053 mAb heavy chain without LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - DNA, 1335 bp, synthetic construct
  misc_feature 1..1335: note = FS28-024-053 mAb heavy chain without LALA
  source 1..1335: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctggtgttcgactactggggccagggaaccctggtcaccgtctcgtcggctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 47 - protein, 445 aa, synthetic construct
  REGION 1..445: note = FS28-024-053 mAb heavy chain with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - DNA, 1335 bp, synthetic construct
  misc_feature 1..1335: note = FS28-024-053 mAb heavy chain with LALA
  source 1..1335: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctggtgttcgactactggggccagggaaccctggtcaccgtctcgtcggctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 49 - protein, 116 aa, synthetic construct
  REGION 1..116: note = FS28-024-053 mAb variable domain
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSS

SEQ ID NO: 50 - DNA, 348 bp, synthetic construct
  misc_feature 1..348: note = FS28-024-053 mAb variable domain
  source 1..348: mol_type = other DNA
gaagtgcaactgctggagtccggtggtggtctggtacagccgggtggttctctgcgtctgagttgcgcggccagtggctttaccctcagttattcttctatgtcatgggtgcgtcaggctccgggcaaaggtctggaatgggttagctttattactccgtctactggctatacccactatgcggatagcgtgaaaggccgttttaccatttctcgcgacaacagcaagaacacgctgtacctgcagatgaactcactgcgtgccgaagatacggccgtgtattactgtgcgagacgggcgctggtgttcgactactggggccagggaaccctggtcaccgtctcgtcg

SEQ ID NO: 51 - protein, 9 aa, synthetic construct
  REGION 1..9: note = FS28-024-053 mAb CDR3 (IMGT)
ARRALVFDY

SEQ ID NO: 52 - protein, 7 aa, synthetic construct
  REGION 1..7: note = FS28-024-053 mAb CDR3 (Kabat)
RALVFDY

SEQ ID NO: 53 - protein, 108 aa, synthetic construct
  REGION 1..108: note = FS28-256-005 mAb variable domain
EIVLTQSPGTLSLSPGERATLSCRASQSVSSSYLAWYQQKPGQAPRLLIYGASSRATGIPDRFSGSGSGTDFTLTISRLEPEDFAVYYCQQALGYPHTFGQGTKVEIK

SEQ ID NO: 54 - DNA, 324 bp, synthetic construct
  misc_feature 1..324: note = FS28-256-005 mAb variable domain
  source 1..324: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaagctttgggttatcctcatacgttcggccaagggaccaaggtggaaatcaaa

SEQ ID NO: 55 - protein, 445 aa, synthetic construct
  REGION 1..445: note = FS28-024-060 mAb heavy chain with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 108 aa, synthetic construct
  REGION 1..108: note = FS28-256-027 mAb variable domain
EIVLTQSPGTLSLSPGERATLSCRASQSVSSSYLAWYQQKPGQAPRLLIYGASSRATGIPDRFSGSGSGTDFTLTISRLEPEDFAVYYCQQTVPYPYTFGQGTKVEIK

SEQ ID NO: 57 - DNA, 324 bp, synthetic construct
  misc_feature 1..324: note = FS28-256-027 mAb variable domain
  source 1..324: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaaactgtgccgtatccgtatacgttcggccaagggaccaaggtggaaatcaaa

SEQ ID NO: 58 - protein, 445 aa, synthetic construct
  REGION 1..445: note = FS28-026 mAb (with LALA) heavy chain
EVQLLESGGGLVQPGGSLRLSCAASGFTFSNYAMTWVRQAPGKGLEWVSSITPYYSKTDYADSVKGRFTISRDNSKNTLYLQMNSLRAEDTAVYYCARNWYRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 215 aa, synthetic construct
  REGION 1..215: note = FS28-026 mAb light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYSSYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 445 aa, synthetic construct
  REGION 1..445: note = FS28-091 mAb heavy chain with LALA
EVQLLESGGGLVQPGGSLRLSCAASGFTFTNYAMSWVRQAPGKGLEWVSSIKPYDGNTYYADSVKGRFTISRDNSKNTLYLQMNSLRAEDTAVYYCARNRWVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 215 aa, synthetic construct
  REGION 1..215: note = FS28-091 mAb light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 452 aa, synthetic construct
  REGION 1..452: note = FS28-185 mAb heavy chain with LALA
EVQLLESGGGLVQPGGSLRLSCAASGFTFTTSAMSWVRQAPGKGLEWVSRINPYEGETNYADSVKGRFTISRDNSKNTLYLQMNSLRAEDTAVYYCARGWSIATYYKSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 119 aa, synthetic construct
  REGION 1..119: note = FS28-256-001 mAb variable domain
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSS

SEQ ID NO: 64 - DNA, 357 bp, synthetic construct
  misc_feature 1..357: note = FS28-256-001 mAb variable domain
  source 1..357: mol_type = other DNA
gaagtgcaactgctggagtccggtggtggtctggtacagccgggtggttctctgcgtctgagttgcgcggccagtggctttaccttcactgagacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaactcttaccagggtggcttggactactggggccagggaaccttggtcaccgtctcgagt

SEQ ID NO: 65 - protein, 448 aa, synthetic construct
  REGION 1..448: note = FS28-256 mAb heavy chain without LALA
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - DNA, 1344 bp, synthetic construct
  misc_feature 1..1344: note = FS28-256 mAb heavy chain without LALA
  source 1..1344: mol_type = other DNA
gaagtgcaactgctggagtccggtggtggtctggtacagccgggtggttctctgcgtctgagttgcgcggccagtggctttaccttcactaacacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaactcttaccagggtggcttggactactggggccagggaaccttggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 67 - protein, 448 aa, synthetic construct
  REGION 1..448: note = FS28-256 mAb heavy chain with LALA
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - DNA, 1344 bp, synthetic construct
  misc_feature 1..1344: note = FS28-256 mAb heavy chain with LALA
  source 1..1344: mol_type = other DNA
gaagtgcaactgctggagtccggtggtggtctggtacagccgggtggttctctgcgtctgagttgcgcggccagtggctttaccttcactaacacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaactcttaccagggtggcttggactactggggccagggaaccttggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 69 - protein, 119 aa, synthetic construct
  REGION 1..119: note = FS28-256 mAb variable domain
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSS

SEQ ID NO: 70 - DNA, 357 bp, synthetic construct
  misc_feature 1..357: note = FS28-256 mAb variable domain
  source 1..357: mol_type = other DNA
gaagtgcaactgctggagtccggtggtggtctggtacagccgggtggttctctgcgtctgagttgcgcggccagtggctttaccttcactaacacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaactcttaccagggtggcttggactactggggccagggaaccttggtcaccgtctcgagt

SEQ ID NO: 71 - protein, 8 aa, synthetic construct
  REGION 1..8: note = FS28-256 mAb CDR1 (IMGT)
GFTFTNTY

SEQ ID NO: 72 - protein, 5 aa, synthetic construct
  REGION 1..5: note = FS28-256 mAb CDR1 (Kabat)
NTYMS

SEQ ID NO: 73 - protein, 8 aa, synthetic construct
  REGION 1..8: note = FS28-256 mAb CDR2 (IMGT)
ISPTYSTT

SEQ ID NO: 74 - protein, 17 aa, synthetic construct
  REGION 1..17: note = FS28-256 mAb CDR2 (Kabat)
NISPTYSTTNYADSVKG

SEQ ID NO: 75 - protein, 12 aa, synthetic construct
  REGION 1..12: note = FS28-256 mAb CDR3 (IMGT)
ARYNSYQGGLDY

SEQ ID NO: 76 - protein, 10 aa, synthetic construct
  REGION 1..10: note = FS28-256 mAb CDR3 (Kabat)
YNSYQGGLDY

SEQ ID NO: 77 - protein, 215 aa, synthetic construct
  REGION 1..215: note = FS28-256 mAb light chain
EIVLTQSPGTLSLSPGERATLSCRASQSVSSSYLAWYQQKPGQAPRLLIYGASSRATGIPDRFSGSGSGTDFTLTISRLEPEDFAVYYCQQSYYYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - DNA, 645 bp, synthetic construct
  misc_feature 1..645: note = FS28-256 mAb light chain
  source 1..645: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatcttattattatcctatcacgttcggccaagggaccaaggtggaaatcaaacgtactgtggccgctcctagcgtgttcatttttccgccatccgacgagcagctcaagtccggcaccgcctccgtggtctgcctgctcaacaacttctaccctcgcgaagctaaggtccagtggaaggtcgacaatgccctgcagtccggaaactcgcaggaaagcgtgactgaacaggactccaaggactccacctattcactgtcctcgactctgaccctgagcaaggcggattacgaaaagcacaaagtgtacgcatgcgaagtgacccaccagggtctttcgtcccccgtgaccaagagcttcaacagaggagagtgt

SEQ ID NO: 79 - protein, 108 aa, synthetic construct
  REGION 1..108: note = FS28-256 mAb variable domain
EIVLTQSPGTLSLSPGERATLSCRASQSVSSSYLAWYQQKPGQAPRLLIYGASSRATGIPDRFSGSGSGTDFTLTISRLEPEDFAVYYCQQSYYYPITFGQGTKVEIK

SEQ ID NO: 80 - DNA, 324 bp, synthetic construct
  misc_feature 1..324: note = FS28-256 mAb variable domain
  source 1..324: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatcttattattatcctatcacgttcggccaagggaccaaggtggaaatcaaa

SEQ ID NO: 81 - protein, 448 aa, synthetic construct
  REGION 1..448: note = FS28-256-001 mAb heavy chain without LALA
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - DNA, 1344 bp, synthetic construct
  misc_feature 1..1344: note = FS28-256-001 mAb heavy chain without LALA
  source 1..1344: mol_type = other DNA
gaagtgcaactgctggagtccggtggtggtctggtacagccgggtggttctctgcgtctgagttgcgcggccagtggctttaccttcactgagacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaactcttaccagggtggcttggactactggggccagggaaccttggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 83 - protein, 215 aa, synthetic construct
  REGION 1..215: note = FS28-256-001 mAb light chain
EIVLTQSPGTLSLSPGERATLSCRASQSVSSSYLAWYQQKPGQAPRLLIYGASSRATGIPDRFSGSGSGTDFTLTISRLEPEDFAVYYCQQHNQYPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - DNA, 1344 bp, synthetic construct
  misc_feature 1..1344: note = FS28-256-001 mAb heavy chain with LALA
  source 1..1344: mol_type = other DNA
gaagtgcaactgctggagtccggtggtggtctggtacagccgggtggttctctgcgtctgagttgcgcggccagtggctttaccttcactgagacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaactcttaccagggtggcttggactactggggccagggaaccttggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 85 - protein, 8 aa, synthetic construct
  REGION 1..8: note = FS28-256-001 mAb CDR1 (IMGT)
GFTFTETY

SEQ ID NO: 86 - protein, 5 aa, synthetic construct
  REGION 1..5: note = FS28-256-001 mAb CDR1 (Kabat)
ETYMS

SEQ ID NO: 87 - protein, 448 aa, synthetic construct; identical to SEQ ID NO: 81
  REGION 1..448: note = FS28-256-005 mAb heavy chain without LALA

SEQ ID NO: 88 - DNA, 1344 bp, synthetic construct; identical to SEQ ID NO: 82
  misc_feature 1..1344: note = FS28-256-005 mAb heavy chain without LALA
  source 1..1344: mol_type = other DNA

SEQ ID NO: 89 - protein, 448 aa, synthetic construct
  REGION 1..448: note = FS28-256-005 mAb heavy chain with LALA
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 215 aa, synthetic construct
  REGION 1..215: note = FS28-256-005 mAb light chain
EIVLTQSPGTLSLSPGERATLSCRASQSVSSSYLAWYQQKPGQAPRLLIYGASSRATGIPDRFSGSGSGTDFTLTISRLEPEDFAVYYCQQALGYPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 645 bp, synthetic construct
  misc_feature 1..645: note = FS28-256-005 mAb light chain
  source 1..645: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaagctttgggttatcctcatacgttcggccaagggaccaaggtggaaatcaaacgtactgtagcagctccttccgtgttcatctttccgcccagtgatgagcagctgaagtcaggtactgcttccgtggtttgcctgctcaacaacttttaccccagagaagccaaagtccagtggaaagtggacaatgcgttgcaaagcgggaactctcaggaatccgtcacagagcaggactctaaggactccacctatagcctctctagtacgctgacactgagcaaagccgattacgagaagcacaaggtgtatgcctgtgaggttacccatcaaggccttagctcaccagtgaccaagagcttcaataggggagaatgc

SEQ ID NO: 92 - DNA, 645 bp, synthetic construct
  misc_feature 1..645: note = FS28-256-021 mAb light chain
  source 1..645: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaacataatcagtatccgaatacgttcggccaagggaccaaggtggaaatcaaacgtactgtagcagctccttccgtgttcatctttccgcccagtgatgagcagctgaagtcaggtactgcttccgtggtttgcctgctcaacaacttttaccccagagaagccaaagtccagtggaaagtggacaatgcgttgcaaagcgggaactctcaggaatccgtcacagagcaggactctaaggactccacctatagcctctctagtacgctgacactgagcaaagccgattacgagaagcacaaggtgtatgcctgtgaggttacccatcaaggccttagctcaccagtgaccaagagcttcaataggggagaatgc

SEQ ID NO: 93 - protein, 108 aa, synthetic construct
  REGION 1..108: note = FS28-256-021 mAb variable domain
EIVLTQSPGTLSLSPGERATLSCRASQSVSSSYLAWYQQKPGQAPRLLIYGASSRATGIPDRFSGSGSGTDFTLTISRLEPEDFAVYYCQQHNQYPNTFGQGTKVEIK

SEQ ID NO: 94 - DNA, 324 bp, synthetic construct
  misc_feature 1..324: note = FS28-256-021 mAb variable domain
  source 1..324: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaacataatcagtatccgaatacgttcggccaagggaccaaggtggaaatcaaa

SEQ ID NO: 95 - protein, 215 aa, synthetic construct
  REGION 1..215: note = FS28-256-027 mAb light chain
EIVLTQSPGTLSLSPGERATLSCRASQSVSSSYLAWYQQKPGQAPRLLIYGASSRATGIPDRFSGSGSGTDFTLTISRLEPEDFAVYYCQQTVPYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - DNA, 645 bp, synthetic construct
  misc_feature 1..645: note = FS28-256-027 mAb light chain
  source 1..645: mol_type = other DNA
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaaactgtgccgtatccgtatacgttcggccaagggaccaaggtggaaatcaaacgtactgtggccgctcctagcgtgttcatttttccgccatccgacgagcagctcaagtccggcaccgcctccgtggtctgcctgctcaacaacttctaccctcgcgaagctaaggtccagtggaaggtcgacaatgccctgcagtccggaaactcgcaggaaagcgtgactgaacaggactccaaggactccacctattcactgtcctcgactctgaccctgagcaaggcggattacgaaaagcacaaagtgtacgcatgcgaagtgacccaccagggtctttcgtcccccgtgaccaagagcttcaacagaggagagtgt

SEQ ID NO: 97 - protein, 5 aa, synthetic construct
  REGION 1..5: note = FS28-256-271 mAb CDR1 (Kabat)
HTYMS

SEQ ID NO: 98 - protein, 8 aa, synthetic construct
  REGION 1..8: note = FS28-256-271 mAb CDR1 (IMGT)
GFTFTHTY

SEQ ID NO: 99 - protein, 12 aa, synthetic construct
  REGION 1..12: note = FS28-256-271 mAb CDR3 (IMGT)
ARYNAYHAALDY

SEQ ID NO: 100 - protein, 10 aa, synthetic construct
  REGION 1..10: note = FS28-256-271 mAb CDR3 (Kabat)
YNAYHAALDY

SEQ ID NO: 101 - protein, 8 aa, synthetic construct
  REGION 1..8: note = FS28-256-018 mAb CDR1 (IMGT)
GFTFTQTY

SEQ ID NO: 102 - protein, 5 aa, synthetic construct
  REGION 1..5: note = FS28-256-018 mAb CDR1 (Kabat)
QTYMS

SEQ ID NO: 103 - protein, 12 aa, synthetic construct
  REGION 1..12: note = FS28-256-018 mAb CDR3 (IMGT)
ARYNAYQIGLDY

SEQ ID NO: 104 - protein, 10 aa, synthetic construct
  REGION 1..10: note = FS28-256-018 mAb CDR3 (Kabat)
YNAYQIGLDY

SEQ ID NO: 105 - protein, 448 aa, synthetic construct
  REGION 1..448: note = FS28-256-021 mAb heavy chain without LALA
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - DNA, 1344 bp, synthetic construct
  misc_feature 1..1344: note = FS28-256-021 mAb heavy chain without LALA
  source 1..1344: mol_type = other DNA
gaagtgcaactgctggagtccggtggtggtctggtacagccgggtggttctctgcgtctgagttgcgcggccagtggctttaccttcactcatacttatatgagctgggtgcgtcaggctccgggcaaaggtctggaatgggttagcaatatttctccgacttatagcactaccaactatgcggatagcgtgaaaggccgttttaccatttctcgcgacaacaacaagaacacgctgtacctgcagatgaactcactgcgtgccgaagatacggccgtgtattactgtgcgagatacaacgcgtatcatgctgctctggactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 107 - protein, 448 aa, synthetic construct
  REGION 1..448: note = FS28-256-021 mAb heavy chain with LALA
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - DNA, 1344 bp, synthetic construct
  misc_feature 1..1344: note = FS28-256-021 mAb heavy chain with LALA
  source 1..1344: mol_type = other DNA
gaagtgcaactgctggagtccggtggtggtctggtacagccgggtggttctctgcgtctgagttgcgcggccagtggctttaccttcactcatacttatatgagctgggtgcgtcaggctccgggcaaaggtctggaatgggttagcaatatttctccgacttatagcactaccaactatgcggatagcgtgaaaggccgttttaccatttctcgcgacaacaacaagaacacgctgtacctgcagatgaactcactgcgtgccgaagatacggccgtgtattactgtgcgagatacaacgcgtatcatgctgctctggactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 109 - protein, 119 aa, synthetic construct
  REGION 1..119: note = FS28-256-021 mAb variable domain
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSS

SEQ ID NO: 110 - DNA, 357 bp, synthetic construct
  misc_feature 1..357: note = FS28-256-021 mAb variable domain
  source 1..357: mol_type = other DNA
gaagtgcaactgctggagtccggtggtggtctggtacagccgggtggttctctgcgtctgagttgcgcggccagtggctttaccttcactcatacttatatgagctgggtgcgtcaggctccgggcaaaggtctggaatgggttagcaatatttctccgacttatagcactaccaactatgcggatagcgtgaaaggccgttttaccatttctcgcgacaacaacaagaacacgctgtacctgcagatgaactcactgcgtgccgaagatacggccgtgtattactgtgcgagatacaacgcgtatcatgctgctctggactactggggccagggaaccctggtcaccgtctcgagt

SEQ ID NO: 111 - protein, 8 aa, synthetic construct
  REGION 1..8: note = FS28-256-014 mAb CDR1 (IMGT)
GFTFTDTY

SEQ ID NO: 112 - protein, 5 aa, synthetic construct
  REGION 1..5: note = FS28-256-014 mAb CDR1 (Kabat)
DTYMS

SEQ ID NO: 113 - protein, 12 aa, synthetic construct
  REGION 1..12: note = FS28-256-014 mAb CDR3 (IMGT)
ARYNAYAAGLDY

SEQ ID NO: 114 - protein, 10 aa, synthetic construct
  REGION 1..10: note = FS28-256-014 mAb CDR3 (Kabat)
YNAYAAGLDY

SEQ ID NO: 115 - protein, 119 aa, synthetic construct
  REGION 1..119: note = FS28-256-014 mAb variable domain
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSS

SEQ ID NO: 116 - DNA, 357 bp, synthetic construct
  misc_feature 1..357: note = FS28-256-014 mAb variable domain
  source 1..357: mol_type = other DNA
gaagtgcaactgctggagtccggtggtggtctggtacagccgggtggttctctgcgtctgagttgcgcggccagtggctttaccttcactgatacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaacgcgtatgcggcgggtcttgactactggggccagggaaccctggtcaccgtctcgagt

SEQ ID NO: 117 - protein, 448 aa, synthetic construct
  REGION 1..448: note = FS28-256-014 mAb heavy chain without LALA
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - DNA, 1344 bp, synthetic construct
  misc_feature 1..1344: note = FS28-256-014 mAb heavy chain without LALA
  source 1..1344: mol_type = other DNA
gaagtgcaactgctggagtccggtggtggtctggtacagccgggtggttctctgcgtctgagttgcgcggccagtggctttaccttcactgatacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaacgcgtatgcggcgggtcttgactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 119 - protein, 448 aa, synthetic construct
  REGION 1..448: note = FS28-256-014 mAb heavy chain with LALA
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - DNA, 1344 bp, synthetic construct
  misc_feature 1..1344: note = FS28-256-014 mAb heavy chain with LALA
  source 1..1344: mol_type = other DNA
gaagtgcaactgctggagtccggtggtggtctggtacagccgggtggttctctgcgtctgagttgcgcggccagtggctttaccttcactgatacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaacgcgtatgcggcgggtcttgactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 121 - protein, 119 aa, synthetic construct
  REGION 1..119: note = FS28-256-018 mAb variable domain
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSS

SEQ ID NO: 122 - DNA, 357 bp, synthetic construct
  misc_feature 1..357: note = FS28-256-018 mAb variable domain
  source 1..357: mol_type = other DNA
gaagtgcaactgctggagtccggtggtggtctggtacagccgggtggttctctgcgtctgagttgcgcggccagtggctttaccttcactcagacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaacgcttatcagattgggttggactactggggccagggaaccctggtcaccgtctcgagt

SEQ ID NO: 123 - protein, 448 aa, synthetic construct
  REGION 1..448: note = FS28-256-023 mAb heavy chain without LALA
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - DNA, 1344 bp, synthetic construct
  misc_feature 1..1344: note = FS28-256-023 mAb heavy chain without LALA
  source 1..1344: mol_type = other DNA
gaagtgcaactgctggagtccggtggtggtctggtacagccgggtggttctctgcgtctgagttgcgcggccagtggctttaccttcactcagacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaacgcttatcagattgggttggactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 125 - protein, 448 aa, synthetic construct
  REGION 1..448: note = FS28-256-023 mAb heavy chain with LALA
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - DNA, 1344 bp, synthetic construct
  misc_feature 1..1344: note = FS28-256-023 mAb heavy chain with LALA
  source 1..1344: mol_type = other DNA
gaagtgcaactgctggagtccggtggtggtctggtacagccgggtggttctctgcgtctgagttgcgcggccagtggctttaccttcactcagacttatatgagctgggtgcgtcaggctccgggcaaaggtctggaatgggttagcaatatttctccgacttatagcactaccaactatgcggatagcgtgaaaggccgttttaccatttctcgcgacaacagcaagaacacgctgtacctgcagatgaactcactgcgtgccgaagatacggccgtgtattactgtgcgagatacaacgcttatcagattgggttggactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 127 - protein, 453 aa, synthetic construct
  REGION 1..453: note = FS22-053-008/FS28-256-012 mAb2 heavy chain without LALA
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 128 - protein, 453 aa, synthetic construct
  REGION 1..453: note = FS22-053-008/FS28-256-012 mAb2 heavy chain with LALA
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 129 - protein, 453 aa, synthetic construct
  REGION 1..453: note = FS22-053-008/FS28-256-014 mAb2 heavy chain without LALA
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 130 - protein, 453 aa, synthetic construct
  REGION 1..453: note = FS22-053-008/FS28-256-014 mAb2 heavy chain with LALA
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 131 - protein, 453 aa, synthetic construct
  REGION 1..453: note = FS22-053-008/FS28-256-018 mAb2 heavy chain without LALA
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 132 - protein, 453 aa, synthetic construct
  REGION 1..453: note = FS22-053-008/FS28-256-018 mAb2 heavy chain with LALA
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 133 - protein, 453 aa, synthetic construct; identical to SEQ ID NO: 127
  REGION 1..453: note = FS22-053-008/FS28-256-021 mAb2 heavy chain without LALA

SEQ ID NO: 134 - protein, 453 aa, synthetic construct; identical to SEQ ID NO: 128
  REGION 1..453: note = FS22-053-008/FS28-256-021 mAb2 heavy chain with LALA

SEQ ID NO: 135 - protein, 453 aa, synthetic construct; identical to SEQ ID NO: 131
  REGION 1..453: note = FS22-053-008/FS28-256-023 mAb2 heavy chain without LALA

SEQ ID NO: 136 - protein, 453 aa, synthetic construct; identical to SEQ ID NO: 132
  REGION 1..453: note = FS22-053-008/FS28-256-023 mAb2 heavy chain with LALA

SEQ ID NO: 137 - protein, 453 aa, synthetic construct; identical to SEQ ID NO: 127
  REGION 1..453: note = FS22-053-008/FS28-256-024 mAb2 heavy chain without LALA

SEQ ID NO: 138 - protein, 453 aa, synthetic construct; identical to SEQ ID NO: 128
  REGION 1..453: note = FS22-053-008/FS28-256-024 mAb2 heavy chain with LALA

SEQ ID NO: 139 - protein, 453 aa, synthetic construct; identical to SEQ ID NO: 131
  REGION 1..453: note = FS22-053-008/FS28-256-026 heavy chain without LALA

SEQ ID NO: 140 - protein, 453 aa, synthetic construct; identical to SEQ ID NO: 132
  REGION 1..453: note = FS22-053-008/FS28-256-026 heavy chain without LALA

SEQ ID NO: 141 - protein, 453 aa, synthetic construct; identical to SEQ ID NO: 127
  REGION 1..453: note = FS22-053-008/FS28-256-027 mAb2 heavy chain without LALA

SEQ ID NO: 142 - protein, 453 aa, synthetic construct; identical to SEQ ID NO: 128
  REGION 1..453: note = FS22-053-008/FS28-256-027 mAb2 heavy chain with LALA

SEQ ID NO: 143 - protein, 450 aa, synthetic construct
  REGION 1..450: note = FS22-053-008/FS28-024 mAb2 heavy chain without LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 144 - protein, 450 aa, synthetic construct
  REGION 1..450: note = FS22-053-008/FS28-024 mAb2 heavy chain with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 145 - protein, 450 aa, synthetic construct
  REGION 1..450: note = FS22-053-008/FS28-024-051 mAb2 heavy chain without LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 146 - protein, 450 aa, synthetic construct
  REGION 1..450: note = FS22-053-008/FS28-024-051 mAb2 heavy chain with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 147 - protein, 450 aa, synthetic construct
  REGION 1..450: note = FS22-053-008/FS28-024-052 mAb2 heavy chain (without LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 148 - protein, 450 aa, synthetic construct
  REGION 1..450: note = FS22-053-008/FS28-024-052 mAb2 heavy chain (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 149 - protein, 450 aa, synthetic construct
  REGION 1..450: note = FS22-053-008/FS28-024-053 mAb2 heavy chain without LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 150 - protein, 450 aa, synthetic construct
  REGION 1..450: note = FS22-053-008/FS28-024-053 mAb2 heavy chain with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 151 - protein, 450 aa, synthetic construct
  REGION 1..450: note = FS22-053-008-AA/FS28-024-060 mAb2 heay chain with LALA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 152 - protein, 450 aa, synthetic construct
  REGION 1..450: note = FS22-053-008-AA/FS28-026 mAb2 heavy chain with LALA
EVQLLESGGGLVQPGGSLRLSCAASGFTFSNYAMTWVRQAPGKGLEWVSSITPYYSKTDYADSVKGRFTISRDNSKNTLYLQMNSLRAEDTAVYYCARNWYRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 153 - protein, 450 aa, synthetic construct
  REGION 1..450: note = FS22-053-008-AA/FS28-091 mAb2 heavy chain with LALA
EVQLLESGGGLVQPGGSLRLSCAASGFTFTNYAMSWVRQAPGKGLEWVSSIKPYDGNTYYADSVKGRFTISRDNSKNTLYLQMNSLRAEDTAVYYCARNRWVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 154 - protein, 457 aa, synthetic construct
  REGION 1..457: note = FS22-053-008-AA/FS28-185 mAb2 heavy chain with LALA
EVQLLESGGGLVQPGGSLRLSCAASGFTFTTSAMSWVRQAPGKGLEWVSRINPYEGETNYADSVKGRFTISRDNSKNTLYLQMNSLRAEDTAVYYCARGWSIATYYKSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 155 - protein, 453 aa, synthetic construct
  REGION 1..453: note = FS22-053-008/FS28-256 mAb2 heavy chain without LALA
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 156 - protein, 453 aa, synthetic construct
  REGION 1..453: note = FS22-053-008/FS28-256 mAb2 heavy chain with LALA
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 157 - protein, 453 aa, synthetic construct
  REGION 1..453: note = FS22-053-008/FS28-256-001 mAb2 heavy chain without LALA
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 158 - protein, 453 aa, synthetic construct
  REGION 1..453: note = FS22-053-008/FS28-256-001 mAb2 heavy chain with LALA
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELNPPYLFSNQVSLTCLVKGFYPSDIAVEWESNGQPENNYKTTPPVLDSDGSFFLYSKLTVDYWRWLEGNVFSCSVMHEALHNHYTQKSLSLSPG

SEQ ID NO: 159 - protein, 453 aa, synthetic construct; identical to SEQ ID NO: 157
  REGION 1..453: note = FS22-053-008/FS28-256-005 mAb2 heavy chain without LALA

SEQ ID NO: 160 - protein, 453 aa, synthetic construct; identical to SEQ ID NO: 158
  REGION 1..453: note = FS22-053-008/FS28-256-005 mAb2 heavy chain with LALA

SEQ ID NO: 161 - protein, 447 aa, synthetic construct
  REGION 1..447: note = FS28m-228 mAb (with LALA) heavy chain
EVQLLESGGGLVQPGGSLRLSCAASGFTFSTYFMVWVRQAPGKGLEWVSMISPKSSNTYYADSVKGRFTISRDNSKNTLYLQMNSLRAEDTAVYYCARWFTPARFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 215 aa, synthetic construct
  REGION 1..215: note = FS28m-228 mAb light chain
EIVLTQSPGTLSLSPGERATLSCRASQSVSSSYLAWYQQKPGQAPRLLIYGASSRATGIPDRFSGSGSGTDFTLTISRLEPEDFAVYYCQQPFPFSFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 447 aa, synthetic construct
  REGION 1..447: note = FS22-063-AA/FS28m-228 mAb2 heavy chain with LALA
EVQLLESGGGLVQPGGSLRLSCAASGFTFSTYFMVWVRQAPGKGLEWVSMISPKSSNTYYADSVKGRFTISRDNSKNTLYLQMNSLRAEDTAVYYCARWFTPARFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEPYWSYVSLTCLVKGFYPSDIAVEWESNGQPENNYKTTPPVLDSDGSFFLYSKLTVMNYRWELGNVFSCSVMHEALHNHYTQKSLSLSPG

SEQ ID NO: 164 - protein, 215 aa, synthetic construct; identical to SEQ ID NO: 162
  REGION 1..215: note = FS22-063-AA/FS28m-228 mAb2 light chain

SEQ ID NO: 165 - protein, 445 aa, synthetic construct
  REGION 1..445: note = G1-AA/HelD1.3 mAb heavy chain with LALA
QVQLQESGPGLVRPSQTLSLTCTVSGSTFSGYGVNWVRQPPGRGLEWIGMIWGDGNTDYNSALKSRVTMLVDTSKNQFSLRLSSVTAADTAVYYCARERDYRLDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 214 aa, synthetic construct
  REGION 1..214: note = G1-AA/HelD1.3 mAb light chain
DIQMTQSPASLSASVGETVTITCRASGNIHNYLAWYQQKQGKSPQLLVYNAKTLADGVPSRFSGSGSGTQYSLKINSLQPEDFGSYYCQHFWSTPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 448 aa, synthetic construct
  REGION 1..448: note = G1-AA/SS1 mAb (with LALA) heavy chain
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 213 aa, synthetic construct
  REGION 1..213: note = G1-AA/SS1 mAb light chain
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 303 aa, Homo sapiens
EVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALS

SEQ ID NO: 170 - protein, 303 aa, Macaca fascicularis
DVERTTCPPEKEVHEIDESLIFYKKRELEACVDAALLAAQMDRVDAIPFTYEQLDVLKHKLDELYPQGYPESVIRHLGHLFLKMSPEDIRKWNVTSLETLKALLKVSKGHEMSAQVATLIDRVVVGRGQLDKDTADTLTAFCPGCLCSLSPERLSSVPPSIIGAVRPQDLDTCGPRQLDVLYPKARLAFQNMSGSEYFVKIRPFLGGAPTEDLKALSQQNVSMDLATFMKLRREAVLPLSVAEVQKLLGPHVEGLKVEEQHSPVRDWILKQRQDDLDTLGLGLQGGIPNGYLILDLSVREALS

SEQ ID NO: 171 - protein, 303 aa, Mus musculus
DAEQKACPPGKEPYKVDEDLIFYQNWELEACVDGTMLARQMDLVNEIPFTYEQLSIFKHKLDKTYPQGYPESLIQQLGHFFRYVSPEDIHQWNVTSPDTVKTLLKVSKGQKMNAQAIALVACYLRGGGQLDEDMVKALGDIPLSYLCDFSPQDLHSVPSSVMWLVGPQDLDKCSQRHLGLLYQKACSAFQNVSGLEYFEKIKTFLGGASVKDLRALSQHNVSMDIATFKRLQVDSLVGLSVAEVQKLLGPNIVDLKTEEDKSPVRDWLFRQHQKDLDRLGLGLQGGIPNGYLVLDFNVREAFS

SEQ ID NO: 172 - protein, 107 aa, synthetic construct
  REGION 1..107: note = wild-type CH3 domain
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 110 aa, synthetic construct
  REGION 1..110: note = CH2 domain containing LALA mutation
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 174 - protein, 110 aa, synthetic construct
  REGION 1..110: note = CH2 domain containing LALA-PA mutation
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK

SEQ ID NO: 175 - DNA, 1901 bp, Homo sapiens
  source 1..1901: mol_type = genomic DNA
aagcttgaattcgccgccaccatggccctgcctaccgctaggcctctgctcggatcctgcggcacacctgccctgggaagcctcctgttcctgctgttctccctgggctgggtgcagccctccagaacactggccggcgaaacaggacaagaggctgcccctctcgatggcgtgctcgctaacccccccaacatcagctccctgtcccctaggcagctcctgggctttccctgtgccgaggtcagcggcctctccaccgagagggtgagggagctggctgtggccctggctcagaagaacgtgaaactgagcaccgagcaactcaggtgcctggctcataggctgtccgagccccccgaggatctggatgccctgcctctcgacctgctgctgttcctgaaccccgacgcttttagcggcccccaggcctgcacaaggttcttcagcagaatcaccaaggccaacgtggatctgctgcccagaggcgctcccgagaggcaaagactgctgcccgccgctctcgcctgttggggcgtcagaggatccctgctgagcgaggccgacgtgagagccctgggcggcctggcttgtgatctgcccggcaggtttgtcgctgagagcgccgaagtgctcctgcccagactggtgagctgccctggacctctggaccaggatcaacaggaggccgccagagctgctctgcagggaggaggacccccctacggacctcctagcacctggtccgtgagcacaatggacgccctgagaggcctgctgcctgtgctgggacagcccatcattaggagcattccccagggcattgtggccgcctggagacagaggagcagcagggacccctcctggaggcagcctgagagaacaatcctgaggcccagattcagaagagaggtggagaaaaccgcctgccctagcggcaagaaggccagagagattgacgagagcctgatcttctataaaaagtgggagctcgaagcctgcgtggatgctgccctgctggccacacagatggacagggtgaacgccatccccttcacctacgagcagctggacgtcctgaagcacaagctcgatgagctgtacccccagggctaccccgagtccgtgattcagcatctcggctacctgttcctgaaaatgagccccgaagacatcaggaagtggaacgtgacaagcctggagaccctcaaggccctgctggaagtgaacaagggacacgagatgagcccccaggtggccaccctcatcgacagatttgtgaagggaaggggacagctggataaggacaccctcgacaccctgaccgccttctaccctggatacctctgcagcctgtcccccgaagagctgtccagcgtgcctccctcctccatctgggccgtcagaccccaggatctcgacacatgcgaccccagacagctggatgtgctgtaccccaaggctaggctggccttccagaacatgaacggatccgaatatttcgtcaaaatccagagctttctgggcggagcccccacagaggacctcaaagccctgagccagcagaacgtcagcatggacctggccacctttatgaaactgagaaccgacgccgtcctccctctgacagtggccgaagtgcagaagctcctgggcccccatgtggaaggcctgaaggccgaggagagacacagacccgtgagagactggattctgaggcagaggcaggacgatctggataccctgggcctgggactgcagggcggcattcctaacggatacctggtcctcgacctgagcatgcaggaagccctgagcggcacaccttgtctgctgggacctggccctgtcctcaccgtgctcgctctgctgctggcttccaccctcgcctgatgagcggccgc

SEQ ID NO: 176 - protein, 448 aa, synthetic construct
  REGION 1..448: note = FS28-256-271 mAb heavy chain without LALA
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - DNA, 1344 bp, synthetic construct
  misc_feature 1..1344: note = FS28-256-271 mAb heavy chain without LALA
  source 1..1344: mol_type = other DNA
gaagtgcaactgctggagtccggtggtggtctggtacagccgggtggttctctgcgtctgagttgcgcggccagtggctttaccttcactcatacttatatgagctgggtgcgtcaggctccgggcaaaggtctggaatgggttagcgcgatttctccgacttatagcactaccaactatgcggatagcgtgaaaggccgttttaccatttctcgcgacaacaacaagaacacgctgtacctgcagatgaactcactgcgtgccgaagatacggccgtgtattactgtgcgagatacaacgcgtatcatgctgctctggactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggccagcctcgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggatccttcttcctctacagcaagctcaccgtggacaagagcaggtggcagcaggggaacgtcttctcctgcagcgtgatgcatgaggctctgcacaaccactacacacagaagagcctctccctgtctccgggt

SEQ ID NO: 178 - protein, 448 aa, synthetic construct
  REGION 1..448: note = FS28-256-271 mAb heavy chain with LALA
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - DNA, 1344 bp, synthetic construct
  misc_feature 1..1344: note = FS28-256-271 mAb heavy chain with LALA
  source 1..1344: mol_type = other DNA
gaagtgcaactgctggagtccggtggtggtctggtacagccgggtggttctctgcgtctgagttgcgcggccagtggctttaccttcactcatacttatatgagctgggtgcgtcaggctccgggcaaaggtctggaatgggttagcgcgatttctccgacttatagcactaccaactatgcggatagcgtgaaaggccgttttaccatttctcgcgacaacaacaagaacacgctgtacctgcagatgaactcactgcgtgccgaagatacggccgtgtattactgtgcgagatacaacgcgtatcatgctgctctggactactggggccagggaaccctggtcaccgtctcgagtgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggccagcctcgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggatccttcttcctctacagcaagctcaccgtggacaagagcaggtggcagcaggggaacgtcttctcctgcagcgtgatgcatgaggctctgcacaaccactacacacagaagagcctctccctgtctccgggt

SEQ ID NO: 180 - protein, 119 aa, synthetic construct
  REGION 1..119: note = FS28-256-271 mAb variable domain
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSS

SEQ ID NO: 181 - DNA, 357 bp, synthetic construct
  misc_feature 1..357: note = FS28-256-271 mAb variable domain
  source 1..357: mol_type = other DNA
gaagtgcaactgctggagtccggtggtggtctggtacagccgggtggttctctgcgtctgagttgcgcggccagtggctttaccttcactcatacttatatgagctgggtgcgtcaggctccgggcaaaggtctggaatgggttagcgcgatttctccgacttatagcactaccaactatgcggatagcgtgaaaggccgttttaccatttctcgcgacaacaacaagaacacgctgtacctgcagatgaactcactgcgtgccgaagatacggccgtgtattactgtgcgagatacaacgcgtatcatgctgctctggactactggggccagggaaccctggtcaccgtctcgagt

SEQ ID NO: 182 - protein, 17 aa, synthetic construct
  REGION 1..17: note = FS28-256-271 mAb CDR2 (Kabat)
AISPTYSTTNYADSVKG

SEQ ID NO: 183-186 - skipped (4 entries)

SEQ ID NO: 187 - protein, 453 aa, synthetic construct
  REGION 1..453: note = FS22-172-003-AA/FS28-256-271 mAb2 heavy chain with LALA
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 188 - protein, 215 aa, synthetic construct; identical to SEQ ID NO: 95
  REGION 1..215: note = FS22-172-003-AA/FS28-256-271 mAb2 light chain

SEQ ID NO: 189 - protein, 448 aa, synthetic construct
  REGION 1..448: note = OX40 (FS20-022-049)/FS28-256-271 mAb2 heavy chain with LALA
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEYWDQEVSLTCLVKGFYPSDIAVEWESNGDEQFAYKTTPPVLDSDGSFFLYSKLTVDQYRWNPADYFSCSVMHEALHNHYTQKSLSLSPG

SEQ ID NO: 190 - protein, 215 aa, synthetic construct; identical to SEQ ID NO: 95
  REGION 1..215: note = OX40 (FS20-022-049)/FS28-256-271 mAb2 light chain

SEQ ID NO: 191 - protein, 445 aa, synthetic construct
  REGION 1..445: note = FS22m-063-AA/HelD1.3 mAb2 heavy chain with LALA
QVQLQESGPGLVRPSQTLSLTCTVSGSTFSGYGVNWVRQPPGRGLEWIGMIWGDGNTDYNSALKSRVTMLVDTSKNQFSLRLSSVTAADTAVYYCARERDYRLDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEPYWSYVSLTCLVKGFYPSDIAVEWESNGQPENNYKTTPPVLDSDGSFFLYSKLTVMNYRWELGNVFSCSVMHEALHNHYTQKSLSLSPG

SEQ ID NO: 192 - protein, 214 aa, synthetic construct; identical to SEQ ID NO: 166
  REGION 1..214: note = FS22m-063-AA/HelD1.3 mAb2 light chain

SEQ ID NO: 193 - protein, 447 aa, synthetic construct
  REGION 1..447: note = FS22m-063-AA/4420 mAb2 heavy chain with LALA
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEPYWSYVSLTCLVKGFYPSDIAVEWESNGQPENNYKTTPPVLDSDGSFFLYSKLTVMNYRWELGNVFSCSVMHEALHNHYTQKSLSLSPG

SEQ ID NO: 194 - protein, 219 aa, synthetic construct
  REGION 1..219: note = FS22m-063-AA/4420 mAb2 light chain
DVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 216 aa, synthetic construct
  REGION 1..216: note = FS28-185 mAb light chain
EIVLTQSPGTLSLSPGERATLSCRASQSVSSSYLAWYQQKPGQAPRLLIYGASSRATGIPDRFSGSGSGTDFTLTISRLEPEDFAVYYCQQSSYSAPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 447 aa, synthetic construct
  REGION 1..447: note = FS22m-063-AA/FS28m-228-010 mAb2 heavy chain with LALA
EVQLLESGGGLVQPGGSLRLSCAASGFTFSTYFMVWVRQAPGKGLEWVSMISPKSSNTYYADSVKGRFTISRDNSKNTLYLQMNSLRAEDTAVYYCARYHISPRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEPYWSYVSLTCLVKGFYPSDIAVEWESNGQPENNYKTTPPVLDSDGSFFLYSKLTVMNYRWELGNVFSCSVMHEALHNHYTQKSLSLSPG

SEQ ID NO: 197 - protein, 215 aa, synthetic construct; identical to SEQ ID NO: 162
  REGION 1..215: note = FS22m-063-AA/FS28m-228-010 mAb2 light chain

SEQ ID NO: 198 - protein, 7 aa, synthetic construct
  REGION 1..7: note = Fcab FS22-172-003 CH3 domain structural loop sequence
PYIIPPY

SEQ ID NO: 199 - protein, 7 aa, synthetic construct
  REGION 1..7: note = Fcab FS22-172-003 CH3 domain structural loop sequence
GADRWLE

SEQ ID NO: 200 - protein, 217 aa, synthetic construct
  REGION 1..217: note = FS22-172-003-AA/FS28-256-271 light chain
AAEIVLTQSPGTLSLSPGERATLSCRASQSVSSSYLAWYQQKPGQAPRLLIYGASSRATGIPDRFSGSGSGTDFTLTISRLEPEDFAVYYCQQTVPYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 453 aa, synthetic construct; identical to SEQ ID NO: 187
  REGION 1..453: note = FS22-172-003-AA/FS28-256-271 heavy chain with LALA

(19) United States
(12) Patent Application Publication
(10) Pub. No.: US 2025/0368730 A1
(43) Pub. Date: Dec. 4, 2025

(54) ANTI-MESOTHELIN ANTIBODIES

(71) Applicant: INVOX PHARMA LIMITED, London (GB)

(72) Inventors: Jose Munoz-Olaya, Cambridge (GB); Remi Fertin, Cambridge (GB); Francisca Wollerton, Cambridge (GB); Mihriban Tuna, Cambridge (GB); Neil Brewis, Cambridge (GB)

(73) Assignee: INVOX PHARMA LIMITED, London (GB)

(21) Appl. No.: 19/197,359

(22) Filed: May 2, 2025

Related U.S. Application Data
(63) Continuation of application No. 17/259,677, filed on Jan. 12, 2021, now Pat. No. 12,325,742.

(30) Foreign Application Priority Data
Jul. 12, 2018 (GB) 1811415.7

(51) Int. Cl. C07K 16/18; A61K 39/00; A61P 35/00; C07K 16/28; G01N 33/574

(52) U.S. Cl. CPC C07K 16/18; A61P 35/00; C07K 16/2878; G01N 33/57496; A61K 2039/505; C07K 2317/31; C07K 2317/33; C07K 2317/524; C07K 2317/526; C07K 2317/55; C07K 2317/565; C07K 2317/732; C07K 2317/76; C07K 2317/92

(57) ABSTRACT

The present application relates to antibody molecules that bind mesothelin (MSLN). The antibody molecules find application in the treatment and diagnosis of diseases and disorders, such as cancer.

### RELATED APPLICATIONS

This application is a continuation of U.S. application Ser. No. 17/259,677, filed Jan. 12, 2021, which is a national stage filing under 35 U.S.C. 371 of International Patent Application No. PCT/EP2019/068800, filed Jul. 12, 2019. The contents of these applications each of which are incorporated herein by reference in their entirety.

### REFERENCE TO AN ELECTRONIC SEQUENCE LISTING

The contents of the electronic sequence listing (F083170007US01-SEQ-ACZ.xml; Size: 328,517 bytes; and Date of Creation: Apr. 30, 2025) are herein incorporated by reference in its entirety.

### FIELD OF THE INVENTION

The present invention relates to antibody molecules that bind mesothelin (MSLN). The antibody molecules find application in the treatment and diagnosis of diseases and disorders, such as cancer.

### BACKGROUND TO THE INVENTION

MSLN is expressed at relatively low levels on mesothelial cells lining the pleura, peritoneum, and pericardium (Hassan et al., 2005) of healthy individuals, but is highly expressed in several different cancers, including mesotheliomas, squamous cell carcinomas, pancreatic cancer, lung, gastric, breast, endometrial and ovarian cancer. The normal biological function of mesothelin is not known. In the context of cancer, high expression levels of MSLN have been correlated with poor prognosis in ovarian cancer, cholangiocarcinoma, lung adenocarcinoma and triple-negative breast cancer. The limited expression of MSLN on normal cells versus the high expression on tumour cells makes it an attractive therapeutic target using monoclonal antibodies (Hassan et al., 2016).

MSLN is expressed as a 69-kDa precursor protein (628 amino acids). The precursor protein is then cleaved by the endoprotease furin to release the secreted N-terminal region called megakaryocyte-potentiating factor (MPF), whereas the 40-kDa protein mature MSLN remains attached to the cell membrane via a glycosylphosphatidylinositol (GPI) linker. Human MSLN shares 60% and 87% amino acid identity with the murine and cynomolgus orthologs of MSLN, respectively.

Membrane bound, mature MSLN is shed from cells as a result of alternative splicing, by creating variants lacking the membrane-anchor sequence, or protease cleavage by tumour necrosis factor α-converting enzyme (TACE) (Sapede et al., 2008, Zhang et al., 2011). Soluble shed MSLN is found in patient's sera and in stroma of tumours including malignant mesothelioma, ovarian cancers or highly metastatic cancers. Measuring soluble MSLN levels in the blood and effusions of mesothelioma patients has been approved by the US FDA for monitoring patient response to treatment and progression (Hollevoet et al., 2012, Creany et al., 2015).

Several antibody-based therapies targeting MSLN have been developed and tested in clinical trials, predominantly in mesothelioma, pancreatic and non-small cell lung cancer (Hassan et al., 2016). The strategies employed include direct tumour cell killing through the use of anti-MSLN antibodies, such as amatuximab, with antibody-dependent cellular cytotoxicity (ADCC) activity, as well as the use of antibody drug conjugates (ADCs), such as SS1P-PE38 and anetumab-ravtansine, comprising an antibody or antibody fragment conjugated to a toxin. In addition, anti-MSLN binding Fv fragments have been used in chimeric antigen receptor T cell therapies and bispecifics, such as ABBV-428, are emerging from preclinical studies to clinical trials.

Amatuximab is a mouse chimeric IgG1 kappa mAb that blocks the MSLN-MUC16 interaction and relies on ADCC function to eliminate tumour cells. Phase I trials showed a good safety profile but limited clinical efficacy when combined with chemotherapy (gemcitabine or pemetrexed/cisplatin) with minimal/no improvement in progression free survival in malignant mesothelioma (NCT00738582, Hassan et al., 2014) and no overall response compared to comparator arm in pancreatic cancer (NCT00570713). SS1-PE-38 is a recombinant immunotoxin, containing the anti-MSLN scFv present in amatuximab, linked to the protein synthesis inhibitor PE38. Despite high anti-tumour activity observed in phase I trials (77% partial response when combined with chemotherapy, NCT01445392), the clinical use of SS1P is limited by immunogenicity and a dose-limiting vascular leak syndrome. LMB-100, an optimised version of SS1P-PE38 with reduced immunogenicity in vitro, is currently being tested in phase I and II trials alone and in combination with chemotherapy (NCT02798536, NCT02810418).

Anti-MSLN targeting is most often used to deliver cytotoxic drugs to tumour cells. Anetumab-ravtansine, a fully human IgG1 covalently linked to the anti-mitotic agent DM4, showed a 31% overall response rate (ORR) in a phase I trials with ovarian, primary peritoneal, fallopian tube cancer and advanced predominantly epithelioid peritoneal mesothelioma (NCT01439152). In mesothelioma, anetumab-ravtansine showed a 50% ORR in combination with standard dosing of chemotherapy (NCT02639091). Like another ADC antibody drug, RG7600, anetumab-ravtansine showed a tolerable safety profile but dose limiting toxicities have been observed in these studies in line with those reported for the respective ADC moieties. To date, phase I data from BMS86148 is not yet available.

In summary, unconjugated antibodies targeting MSLN have shown favourable safety profiles but their therapeutic efficacy has been limited, whereas ADCs have shown more potent in anti-tumour activity but were associated with dose limiting toxicities. With regard to safety, ADCs might have a bigger therapeutic window than the immunotoxin therapies (Zhao et al., 2016). For a number of these antibody-based MSLN therapeutics, phase II clinical trials combining the therapeutic with either chemotherapy or with immune checkpoint inhibitors such as PD-1 or PD-L1 are ongoing. Several bispecific molecules intended to engage the immune system are also in development, including ABBV-428, which targets MSLN as well as the costimulatory protein CD40, the MSLN-CD3 bispecific T cell engager (BITE), and a MSLN-CD47 bispecific molecule.

### STATEMENTS OF INVENTION

As explained above, mature MSLN, like other tumour-associated antigens (TAAs), is known to be shed from the cell surface by enzymatic cleavage. The shed/soluble portion of MSLN is then cleared away from the tumour site. This represents a challenge for therapeutics targeting MSLN, as the shed/soluble portion can act as a sink for the therapeutic, clearing the therapeutic away from the tumour site before it binds to the tumour.

The present inventors conducted an extensive selection program to isolate antibody molecules that bind with higher affinity to immobilised MSLN than to MSLN in solution.

‘Affinity’ as referred to herein may refer to the strength of the binding interaction between an antibody molecule and its cognate antigen as measured by K_{D}. As would be readily apparent to the skilled person, where the antibody molecule is capable of forming multiple binding interactions with an antigen (e.g. where the antibody molecule is capable of binding the antigen bivalently and, optionally, the antigen is dimeric) the affinity, as measured by K_{D}, may also be influenced by avidity, whereby avidity refers to the overall strength of an antibody-antigen complex.

Specifically, it is thought that the antibody molecules of the invention bind to MSLN with high avidity and thus bind MSLN more strongly where the antibody is able to bind to two MSLN molecules, as is the case where multiple copies of the antigen are immobilised at a surface, than where the MSLN is in monomeric form, as is expected to be the case with MSLN in solution. Without wishing to be bound by theory, it is therefore thought that the antibody molecules of the invention will not remain bound to shed MSLN in solution in vivo due to the low affinity of the antibodies for monomeric MSLN, thus will not be cleared from the tumour site as quickly, and hence will have longer to exert their therapeutic effect by binding MSLN on the surface of tumour cells. Preferential targeting of membrane-bound MSLN has been reported previously, although the molecules in question were isolated using different approaches to that employed by the present inventors. Specifically, preferential targeting of membrane-bound MSLN has been reported through isolation of molecules targeting different regions of MSLN (Asgarov et al., 2017; Tang et al., 2013). For example, a single domain (VH domain only) antibody fused to human Fc, SD1-Fc, has been reported which targets an epitope close to the cell membrane to promote CDC activity (Tang et al., 2013). It is however unclear how exposed such epitopes are in different cancer settings. In addition, the MSLN-CD3 bispecific T cell engager (BITE) has also been reported to preferentially bind to cell-bound MSLN. However, neither of these molecules are full IgG molecules and neither is capable of binding to MSLN bivalently as both have monovalent binding to the target (via the VH domain in SD1-Fc or the scFv of the BITE).

The antibody molecules isolated by the present inventors bind different epitopes/regions on MSLN. This evident from the fact that some of the antibody molecules are capable of blocking binding of the ligand MUC16 to MSLN while others are not. Blocking MUC16 to MSLN is thought to be advantageous for inhibiting metastasis of MUC16 expressing cancer cells to MSLN expressing surfaces in the pleura and peritoneum (Chen et al., 2013). Other binding regions closer to the cell membrane might facilitate ADCC and CDC activity.

The anti-MSLN antibody molecules of the invention have been shown to have ADCC activity and thus are expected to find application in cancer treatment. Specifically, the antibody molecules have been shown to be capable of targeting tumour cells comprising MSLN on their cell surface and mediating killing of the tumour cell via ADCC.

The antibody molecules of the invention may also be useful for preparing ADCs comprising the antibody molecule of the invention and a bioactive molecule, such as a toxin. Such molecules are also expected to find application in the treatment of cancers comprising MSLN on their cell surface through targeted delivery of the bioactive molecule to the tumour cell.

The present inventors have recognised that the anti-MSLN antibodies of the invention can be used to prepare multispecific, e.g. bispecific, molecules which bind a second antigen in addition to MSLN. Preferably the multispecific molecule binds the second antigen bivalently. In particular, the present inventors have prepared anti-MSLN antibody molecules comprising an additional antigen-binding site in each of the CH3 domains of the antibody molecule which are able to bind a second antigen bivalently.

The second antigen bound by the antibody molecule may be an immune cell antigen, such as a member of the tumour necrosis factor receptor superfamily (TNFRSF). Tumour necrosis factor (TNF) receptors require clustering for activation. Specifically, initial ligation of a TNF receptor ligand to its receptor initiates a chain of events that leads to TNF receptor trimerisation, followed by receptor clustering, activation the NFkB intracellular signalling pathway and subsequent immune cell activation. For a therapeutic agent to efficiently activate a TNFR receptor, several TNF receptor monomers therefore need to be bridged together in a way that mimics the trimeric ligand. Many anti-TNF receptor agonist antibodies either require crosslinking by Fcγ receptors for their agonist activity or exhibit agonist activity in the absence of crosslinking. In both cases, the agonist activity of the antibody is not limited to a particular site, as Fcγ receptors are found throughout the human body.

Bispecific anti-MSLN antibody molecules comprising constant domain binding sites for an immune cell antigen are expected to be capable of activating the immune cell antigen conditionally in the presence of MSLN without the need for e.g. Fcγ receptor-mediated crosslinking, as required by conventional antibody molecules. It is thought that binding of the antibody molecules to MSLN will cause crosslinking of the antibody molecules at the site of MSLN, which in turn will lead to clustering and activation of immune cell antigen on the immune cell surface. The agonistic activity of the antibody molecules is therefore expected to be dependent on both the immune cell antigen and MSLN being present. In other words, the agonistic activity is expected to be conditional. In addition, crosslinking of the antibodies in the presence of MSLN is thought to assist with clustering of the immune cell antigen bound via the constant domain antigen-binding sites of the antibody molecule. As MSLN is a tumour antigen, the antibody molecules are therefore expected to be capable of activating immune cells in a disease-dependent manner, e.g. in the tumour microenvironment. This targeted activation of immune cells is expected to be beneficial in avoiding off-target side effects. The present inventors have shown that bispecific antibody molecules comprising an anti-MSLN and an anti-CD137 binding site were capable of inhibiting tumour growth and increasing survival in the absence of Fcγ receptor binding in a mouse tumour model.

Antibody molecules comprising an anti-MSLN Fab and CH3 domain binding sites specific for a second antigen bind both MSLN and the second antigen bivalently. Where the second antigen is an immune cell antigen, the bivalent binding of both targets is expected to make the bridging between the immune cell expressing the immune cell antigen and MSLN more stable and thereby extend the time during which the immune cell is localised at a particular site, such as a tumour microenvironment, and can act on the disease, e.g. the tumour. This is different to the vast majority of conventional bispecific antibody formats which are heterodimeric and bind each target antigen monovalently via one Fab arm. Such a monovalent interaction is expected to be not only less stable but in many cases is insufficient to induce clustering of immune cell antigens such as TNF receptors in the first place.

A further feature of the anti-MSLN antibody molecules of the invention comprising CH3 domain binding sites specific for a second antigen is that the two antigen binding sites for MSLN and the second antigen are both contained within the antibody structure itself. In particular, the antibody molecules do not require other proteins to be fused to the antibody molecule via linkers or other means to result in molecule that binds bivalently to both of its targets. This has a number of advantages. Specifically, the antibody molecules can be produced using methods similar to those employed for the production of standard antibodies, as they do not comprise any additional fused portions. The structure is also expected to result in improved antibody stability, as linkers may degrade over time, resulting in a heterogeneous population of antibody molecules. Those antibodies in the population having only one protein fused may not be able to induce conditional agonism of immune cell antigens, such as TNF receptors, as efficiently as those having two fused proteins. Cleavage/degradation of the linker could take place prior to administration or after administration of the therapeutic to the patient (e.g. through enzymatic cleavage or the in vivo pH of the patient), thereby resulting in a reduction of its effectiveness whilst circulating in the patient. As there are no linkers in the antibody molecules, the antibody molecules are expected to retain the same number of binding sites both before and after administration. Furthermore, the structure of the antibody molecules is also preferred from the perspective of immunogenicity of the molecules, as the introduction of fused proteins or linkers or both may induce immunogenicity when the molecules are administered to a patient, resulting in reduced effectiveness of the therapeutic.

Thus, the present invention provides:

[1] An antibody molecule that binds to mesothelin (MSLN), wherein the antigen-binding site of the antibody molecule comprises complementarity determining regions (CDRs) 1-6 of antibody:
(i) FS28-256-271 set forth in SEQ ID NO: 98, 73, 99, 20, 21, and 44, respectively;
(ii) FS28-024-052 set forth in SEQ ID NO: 10, 11, 41, 20, 21 and 22, respectively;
(iii) FS28-256-021 set forth in SEQ ID NO: 98, 73, 99, 20, 21 and 34, respectively;
(iv) FS28-256-012 set forth in SEQ ID NO: 98, 73, 99, 20, 21 and 25, respectively;
(v) FS28-256-023 set forth in SEQ ID NO: 101, 73, 103, 20, 21 and 34, respectively;
(vi) FS28-256-024 set forth in SEQ ID NO: 98, 73, 99, 20, 21 and 43, respectively;
(vii) FS28-256-026 set forth in SEQ ID NO: 101, 73, 103, 20, 21 and 43, respectively;
(viii) FS28-256-027 set forth in SEQ ID NO: 98, 73, 99, 20, 21 and 44, respectively;
(ix) FS28-256-001 set forth in SEQ ID NO: 85, 73, 75, 20, 21 and 34, respectively;
(x) FS28-256-005 set forth in SEQ ID NO: 85, 73, 75, 20, 21 and 43, respectively;
(xi) FS28-256-014 set forth in SEQ ID NO: 111, 73, 113, 20, 21 and 25, respectively;
(xii) FS28-256-018 set forth in SEQ ID NO: 101, 73, 103, 20, 21 and 25, respectively;
(xiii) FS28-256 set forth in SEQ ID NO: 71, 73, 75, 20, 21 and 25, respectively;
(xiv) FS28-024-051 set forth in SEQ ID NO: 10, 11, 32, 20, 21 and 22, respectively;
(xv) FS28-024-053 set forth in SEQ ID NO: 10, 11, 51, 20, 21 and 22, respectively; or
(xvi) FS28-024 set forth in SEQ ID NO: 10, 11, 12, 20, 21 and 22, respectively; and
wherein the CDR sequences are defined according to the ImMunoGeneTics (IMGT) numbering scheme.

[2] An antibody molecule that binds to MSLN, wherein the antigen-binding site of the molecule comprises CDRs 1-6 of antibody:
(i) FS28-256-271 set forth in SEQ ID NO: 97, 182, 100, 23, 24, and 44, respectively;
(ii) FS28-024-052 set forth in SEQ ID NO: 13, 14, 42, 23, 24 and 22, respectively;
(iii) FS28-256-021 set forth in SEQ ID NO: 97, 74, 100, 23, 24 and 34, respectively;
(iv) FS28-256-012 set forth in SEQ ID NO: 97, 74, 100, 23, 24 and 25, respectively;
(v) FS28-256-023 set forth in SEQ ID NO: 102, 74, 104, 23, 24 and 34, respectively;
(vi) FS28-256-024 set forth in SEQ ID NO: 97, 74, 100, 23, 24 and 43, respectively;
(vii) FS28-256-026 set forth in SEQ ID NO: 102, 74, 104, 23, 24 and 43, respectively;
(viii) FS28-256-027 set forth in SEQ ID NO: 97, 74, 100, 23, 24 and 44, respectively;
(ix) FS28-256-001 set forth in SEQ ID NO: 86, 74, 76, 23, 24 and 34, respectively;
(x) FS28-256-005 set forth in SEQ ID NO: 86, 74, 76, 23, 24 and 43, respectively;
(xi) FS28-256-014 set forth in SEQ ID NO: 112, 74, 114, 23, 24 and 25, respectively;
(xii) FS28-256-018 set forth in SEQ ID NO: 102, 74, 104, 23, 24 and 25, respectively;
(xiii) FS28-256 set forth in SEQ ID NO: 72, 74, 76, 23, 24 and 25, respectively;
(xiv) FS28-024-051 set forth in SEQ ID NO: 13, 14, 33, 23, 24 and 22, respectively;
(xiv) FS28-024-053 set forth in SEQ ID NO: 13, 14, 52, 23, 24 and 22, respectively; or
(xvi) FS28-024 set forth in SEQ ID NO: 13, 14, 15, 23, 24 and 22, respectively; and
wherein the CDR sequences are defined according to the Kabat numbering scheme.

[3] The antibody molecule according to [1] or [2], wherein the antibody molecule comprises a heavy chain variable (VH) domain and/or light chain variable (VL) domain, preferably a VH domain and a VL domain.

[4] The antibody molecule according to any one of [1] to [3], wherein the antibody molecule comprises an immunoglobulin heavy chain and/or an immunoglobulin light chain, preferably an immunoglobulin heavy chain and an immunoglobulin light chain.

[5] The antibody molecule according to any one of [3] to [4], wherein the antibody molecule comprises the VH domain and/or VL domain, preferably the VH domain and the VL domain, of antibody:
(i) FS28-256-271 set forth in SEQ ID NO: 180, and 56, respectively;
(ii) FS28-024-052 set forth in SEQ ID NO: 39 and 18, respectively;
(iii) FS28-256-021 set forth in SEQ ID NO: 109 and 93, respectively;
(iv) FS28-256-012 set forth in SEQ ID NO: 109 and 79, respectively;
(v) FS28-256-023 set forth in SEQ ID NO: 121 and 93, respectively;
(vi) FS28-256-024 set forth in SEQ ID NO: 109 and 53, respectively;
(vii) FS28-256-026 set forth in SEQ ID NO: 121 and 53, respectively;
(viii) FS28-256-027 set forth in SEQ ID NO: 109 and 56, respectively;
(ix) FS28-256-001 set forth in SEQ ID NO: 63 and 93, respectively;
(x) FS28-256-005 set forth in SEQ ID NO: 63 and 53, respectively;
(xi) FS28-256-014 set forth in SEQ ID NO: 115 and 79, respectively;
(xii) FS28-256-018 set forth in SEQ ID NO: 121 and 79, respectively;
(xiii) FS28-256 set forth in SEQ ID NO: 69 and 79, respectively;
(xiv) FS28-024-051 set forth in SEQ ID NO: 30 and 18, respectively;
(xv) FS28-024-053 set forth in SEQ ID NO: 49 and 18, respectively; or
(xvi) FS28-024 set forth in SEQ ID NO: 8 and 18, respectively.

[6] The antibody molecule according to any one of [1] to [5], wherein the antibody molecule comprises the heavy chain [without LALA] and light chain of antibody:
(i) FS28-256-271 set forth in SEQ ID NO: 176, and 95, respectively;
(ii) FS28-024-052 set forth in SEQ ID NO: 35 and 16, respectively;
(iii) FS28-256-021 set forth in SEQ ID NO: 105 and 83, respectively;
(iv) FS28-256-012 set forth in SEQ ID NO: 105 and 77, respectively;
(v) FS28-256-023 set forth in SEQ ID NO: 123 and 83, respectively;
(vi) FS28-256-024 set forth in SEQ ID NO: 105 and 90, respectively;
(vii) FS28-256-026 set forth in SEQ ID NO: 123 and 90, respectively;
(viii) FS28-256-027 set forth in SEQ ID NO: 105 and 95, respectively;
(ix) FS28-256-001 set forth in SEQ ID NO: 81 and 83, respectively;
(x) FS28-256-005 set forth in SEQ ID NO: 87 and 90, respectively;
(xi) FS28-256-014 set forth in SEQ ID NO: 117 and 77, respectively;
(xii) FS28-256-018 set forth in SEQ ID NO: 123 and 77, respectively;
(xiii) FS28-256 set forth in SEQ ID NO: 65 and 77, respectively;
(xiv) FS28-024-051 set forth in SEQ ID NO: 26 and 16, respectively;
(xv) FS28-024-053 set forth in SEQ ID NO: 45 and 16, respectively; or
(xvi) FS28-024 set forth in SEQ ID NO: 4 and 16, respectively.

[7] The antibody molecule according to any one of [1] to [5], wherein the antibody molecule comprises the heavy chain [with LALA] and light chain of antibody:
(i) FS28-256-271 set forth in SEQ ID NO: 178, and 95, respectively;
(ii) FS28-024-052 set forth in SEQ ID NO: 37 and 16, respectively;
(iii) FS28-256-021 set forth in SEQ ID NO: 107 and 83, respectively;
(iv) FS28-256-012 set forth in SEQ ID NO: 107 and 77, respectively;
(v) FS28-256-023 set forth in SEQ ID NO: 125 and 83, respectively;
(vi) FS28-256-024 set forth in SEQ ID NO: 107 and 90, respectively;
(vii) FS28-256-026 set forth in SEQ ID NO: 125 and 90, respectively;
(viii) FS28-256-027 set forth in SEQ ID NO: 107 and 95, respectively;
(ix) FS28-256-001 set forth in SEQ ID NO: 89 and 83, respectively;
(x) FS28-256-005 set forth in SEQ ID NO: 89 and 90, respectively;
(xi) FS28-256-014 set forth in SEQ ID NO: 119 and 77, respectively;
(xii) FS28-256-018 set forth in SEQ ID NO: 125 and 77, respectively;
(xiii) FS28-256 set forth in SEQ ID NO: 67 and 77, respectively;
(xiv) FS28-024-051 set forth in SEQ ID NO: 28 and 16, respectively;
(xv) FS28-024-053 set forth in SEQ ID NO: 47 and 16, respectively; or
(xvi) FS28-024 set forth in SEQ ID NO: 5 and 16, respectively.

[8] The antibody molecule according to any one of [1] to [7], wherein the antibody molecule comprises CDRs 1-6, the VH domain, VL domain, light chain and/or heavy chain of antibody FS28-256-271.

[9] The antibody molecule according to any one of [1] to [7], wherein the antibody molecule comprises CDRs 1-6, the VH domain, VL domain, light chain and/or heavy chain of antibody FS28-024-052.

[10] The antibody molecule according to any one of [1] to [9], wherein the MSLN is cell-surface bound MSLN.

[11] The antibody molecule according to [10], wherein the antibody molecule binds to immobilised MSLN with a higher affinity than to MSLN in solution.

[12] The antibody molecule according to [11], wherein the antibody molecule binds to immobilised MSLN with an affinity (kD) of 8 nM or with a higher affinity.

[13] The antibody molecule according to [11] or [12], wherein the antibody molecule binds to MSLN in solution with an affinity (kD) of 15 nM or with a lower affinity.

[14] The antibody molecule according to any one of [1] to [13], wherein the MSLN is human MSLN.

[15] The antibody molecule according to [14], wherein the MSLN consists of or comprises the sequence set forth in SEQ ID NO: 169.

[16] The antibody molecule according to any one of [1] to [13], wherein the MSLN is cynomolgus MSLN.

[17] The antibody molecule according to [16], wherein the MSLN consists of or comprises the sequence set forth in SEQ ID NO: 170.

[18] The antibody molecule according to any one of [1] to [17], wherein the antibody molecule comprises CDRs 1-6, the VH domain, VL domain, light chain and/or heavy chain of antibody FS28-024-051, FS28-024-052, FS28-024-053, or FS28-024, and wherein the antibody blocks binding of MUC16 to MSLN.

[19] The antibody molecule according to any one of [1] to [17], wherein the antibody molecule comprises CDRs 1-6, the VH domain, VL domain, light chain and/or heavy chain of antibody FS28-256-271, FS28-256-021, FS28-256-012, FS28-256-023, FS28-256-024, FS28-256-026, or FS28-256-027, FS28-256-001, FS28-256-005, FS28-256-014, FS28-256-018, or FS28-256, and wherein the antibody does not block binding of MUC16 to MSLN.

[20] The antibody molecule according to [18] or [19], wherein the MUC16 is human MUC16.

[21] The antibody molecule according to any one of [1] to [20], wherein antibody molecule is a multispecific antibody molecule and comprises a second antigen-binding site that binds a second antigen.

[22] The antibody molecule according to [21], wherein antibody molecule is a bispecific, trispecific, or tetraspecific antibody molecule.

[23] The antibody molecule according to [22], wherein antibody molecule is a bispecific molecule.

[24] The antibody molecule according to any one of [21] to [23], wherein the second antigen-binding site is located in a constant domain of the antibody molecule.

[25] The antibody molecule according to any one of [21] to [24], wherein the second antigen is an immune cell antigen.

[26] The antibody molecule according to [25], wherein the immune cell antigen is a member of the tumour necrosis factor receptor superfamily (TNFRSF).

[27] The antibody molecule according to [26], wherein the member of the TNFRSF is OX40 or CD137.

[28] The antibody molecule according to any one of [21] to [27], wherein the second antigen-binding site comprises a first sequence, a second sequence, and/or a third sequence, wherein the first sequence, second sequence and third sequence are located in the AB structural loop, the CD structural loop and the EF structural loop of the constant domain, respectively.

[29] The antibody molecule according to any one of [24] to [28], wherein the constant domain is a CH3 domain.

[30] The antibody molecule according to any one of [26] to [29], wherein the antibody molecule is capable of activating the TNFRSF member on an immune cell in the presence of tumour cell-surface bound MSLN.

[31] The antibody molecule according to any one of [26] to [30] wherein binding of the antibody molecule to the TNFRSF member and to tumour cell-surface bound MSLN, causes clustering of the TNFRSF member on the immune cell surface.

[32] The antibody molecule according to [30] or [31], wherein the immune cell is a T cell, B cell, natural killer (NK) cell, natural killer T (NKT) cell, or dendritic cell (DC).

[33] The antibody molecule according to [32], wherein the immune cell is a T cell.

[34] The antibody molecule according to any one of [1] to [33], wherein the antibody molecule has, or is capable of eliciting, antibody-dependent cellular cytotoxicity (ADCC).

[35] The antibody molecule according to any one of [1] to [33], wherein the antibody molecule has been modified to reduce or abrogate binding of the CH2 domain of the antibody molecule to one or more Fcγ receptors.

[36] The antibody molecule according to any one of [1] to [33] or [35] wherein the antibody molecule does not bind to Fcγ receptors.

[37] The antibody molecule according to [35] or [36], wherein the Fcγ receptor is selected from the group consisting of: FcγRI, FcγRIIa, FcγRIIb and FcγRIII.

[38]A conjugate comprising the antibody molecule according to any one of [1] to [37] and a bioactive molecule.

[39]A conjugate comprising the antibody molecule according to any one of [1] to [37] and a detectable label.

[40]A nucleic acid molecule or molecules encoding the antibody molecule according to any one of [1] to [37].

[41] The nucleic acid molecule(s) according to [40], wherein the nucleic acid molecule(s) comprise(s) the VH domain and/or the VL domain nucleic acid sequence of antibody:
(i) FS28-256-271 set forth in SEQ ID NO: 181, and 57, respectively;
(ii) FS28-024-052 set forth in SEQ ID NO: 40 and 19, respectively;
(iii) FS28-256-021 set forth in SEQ ID NO: 110 and 94, respectively;
(iv) FS28-256-012 set forth in SEQ ID NO: 110 and 80, respectively;
(v) FS28-256-023 set forth in SEQ ID NO: 122 and 94, respectively;
(vi) FS28-256-024 set forth in SEQ ID NO: 110 and 54, respectively;
(vii) FS28-256-026 set forth in SEQ ID NO: 122 and 54, respectively;
(viii) FS28-256-027 set forth in SEQ ID NO: 110 and 57, respectively;
(ix) FS28-256-001 set forth in SEQ ID NO: 64 and 94, respectively;
(x) FS28-256-005 set forth in SEQ ID NO: 64 and 54, respectively;
(xi) FS28-256-014 set forth in SEQ ID NO: 116 and 80, respectively;
(xii) FS28-256-018 set forth in SEQ ID NO: 122 and 80, respectively;
(xiii) FS28-256 set forth in SEQ ID NO: 70 and 80, respectively;
(xiv) FS28-024-051 set forth in SEQ ID NO: 31 and 19, respectively;
(xv) FS28-024-053 set forth in SEQ ID NO: 50 and 19, respectively; or
(xvi) FS28-024 set forth in SEQ ID NO: 9 and 19, respectively.

[42] The nucleic acid molecule(s) according to [40] or [41], wherein the nucleic acid molecule(s) comprise(s):
(i) the heavy chain nucleic acid sequence of antibody FS28-256-271 set forth in SEQ ID NO: 179 or 177, and/or the light chain nucleic acid sequence of antibody FS28-256-271 set forth in SEQ ID NO: 96;
(ii) the heavy chain nucleic acid sequence of antibody FS28-024-052 set forth in SEQ ID NO: 38 or 36, and/or the light chain nucleic acid sequence of antibody FS28-024-052 set forth in SEQ ID NO: 17;
(iii) the heavy chain nucleic acid sequence of antibody FS28-256-021 set forth in SEQ ID NO: 108 or 106, and/or the light chain nucleic acid sequence of antibody FS28-256-021 set forth in SEQ ID NO: 92;
(iv) the heavy chain nucleic acid sequence of antibody FS28-256-012 set forth in SEQ ID NO: 108 or 106, and/or the light chain nucleic acid sequence of antibody FS28-256-012 set forth in SEQ ID NO: 78;
(v) the heavy chain nucleic acid sequence of antibody FS28-256-023 set forth in SEQ ID NO: 126 or 124, and/or the light chain nucleic acid sequence of antibody FS28-256-023 set forth in SEQ ID NO: 92;
(vi) the heavy chain nucleic acid sequence of antibody FS28-256-024 set forth in SEQ ID NO: 108 or 106, and/or the light chain nucleic acid sequence of antibody FS28-256-024 set forth in SEQ ID NO: 91;
(vii) the heavy chain nucleic acid sequence of antibody FS28-256-026 set forth in SEQ ID NO: 126 or 124, and/or the light chain nucleic acid sequence of antibody FS28-256-026 set forth in SEQ ID NO: 91;
(viii) the heavy chain nucleic acid sequence of antibody FS28-256-027 set forth in SEQ ID NO: 108 or 106, and/or the light chain nucleic acid sequence of antibody FS28-256-027 set forth in SEQ ID NO: 96;
(ix) the heavy chain nucleic acid sequence of antibody FS28-256-001 set forth in SEQ ID NO: 84 or 82, and/or the light chain nucleic acid sequence of antibody FS28-256-001 set forth in SEQ ID NO: 92;
(x) the heavy chain nucleic acid sequence of antibody FS28-256-005 set forth in SEQ ID NO: 84 or 88, and/or the light chain nucleic acid sequence of antibody FS28-256-005 set forth in SEQ ID NO: 91;
(xi) the heavy chain nucleic acid sequence of antibody FS28-256-014 set forth in SEQ ID NO: 120 or 118, and/or the light chain nucleic acid sequence of antibody FS28-256-014 set forth in SEQ ID NO: 78;
(xii) the heavy chain nucleic acid sequence of antibody FS28-256-018 set forth in SEQ ID NO: 126 or 124, and/or the light chain nucleic acid sequence of antibody FS28-256-018 set forth in SEQ ID NO: 78;
(xiii) the heavy chain nucleic acid sequence of antibody FS28-256 set forth in SEQ ID NO: 68 or 66, and/or the light chain nucleic acid sequence of antibody FS28-256 set forth in SEQ ID NO: 78;
(xiv) the heavy chain nucleic acid sequence of antibody FS28-024-051 set forth in SEQ ID NO: 29 or 27, and/or the light chain nucleic acid sequence of antibody FS28-024-051 set forth in SEQ ID NO: 17;
(xv) the heavy chain nucleic acid sequence of antibody FS28-024-053 set forth in SEQ ID NO: 48 or 46, and/or the light chain nucleic acid sequence of antibody FS28-024-053 set forth in SEQ ID NO: 17; or
(xvi) the heavy chain nucleic acid sequence of antibody FS28-024 set forth in SEQ ID NO: 7 or 6, and/or the light chain nucleic acid sequence of antibody FS28-024 set forth in SEQ ID NO: 17.

[43]A vector or vectors comprising the nucleic acid molecule or molecules according to any one of [40] to [42].

[44]A recombinant host cell comprising the nucleic acid molecule(s) according to any one of [40] to [42], or the vector(s) according to [43].

[45]A method of producing the antibody molecule according to any one of [1] to [37] comprising culturing the recombinant host cell of [44] under conditions for production of the antibody molecule.

[46] The method according to [45] further comprising isolating and/or purifying the antibody molecule.

[47]A pharmaceutical composition comprising the antibody molecule or conjugate according to any one of [1] to [39] and a pharmaceutically acceptable excipient.

[48] The antibody molecule or conjugate according to any one of [1] to [39] for use in a method of treating cancer in an individual.

[49]A method of treating cancer in an individual comprising administering to the individual a therapeutically effective amount of the antibody molecule or conjugate according to any one of [1] to [39].

[50] The use of the antibody molecule or conjugate according to any one of [1] to [39] in the preparation of a medicament for the treatment of cancer.

[51] The antibody molecule or conjugate for use, the method, or the use according to any one of [48] to [50], wherein cells of the cancer express MSLN on their cell surface.

[52] The antibody molecule or conjugate for use, the method, or the use according to any one of [48] to [50], wherein the cancer is selected from the group consisting of: mesothelioma, pancreatic cancer, ovarian cancer, and lung cancer.

[53] The antibody molecule or conjugate for use according to [48], where the method of treatment comprises administering the antibody molecule or conjugate to the individual in combination with a second therapeutic.

[54] The method according to [49], wherein the method further comprises administering a therapeutically effective amount of a second therapeutic to the individual.

[55] The antibody molecule or conjugate according to any one of [1] to [37] or [39] for use in a method of detecting, diagnosing, prognosis or monitoring the prognosis of cancer in an individual.

[56]A method of detecting, diagnosing, prognosis or monitoring the prognosis of a cancer in an individual, the method comprising the use of the antibody molecule or conjugate according to any one of [1] to [37] or [39].

[57] The use of the antibody molecule or conjugate according to any one of [1] to [37] or [39] in the manufacture of a diagnostic product for detecting, diagnosing, prognosis or monitoring the prognosis of cancer in an individual.

[58]A kit for use in a method of detecting, diagnosing, prognosis, or monitoring the prognosis of cancer in an individual, the kit comprising an antibody molecule or conjugate according to any one of [1] to [37] or [39].

[59] The antibody molecule or conjugate for use, the method, the use, or the kit according to any one of [55] to [58], wherein the cancer is selected from the group consisting of: mesothelioma, pancreatic cancer, ovarian cancer, and lung cancer.

### BRIEF DESCRIPTION OF THE FIGURES

FIG. 1 shows binding of the anti-MSLN mAbs to NCI-H226 cells. The anti-human MSLN mAbs FS28-024, FS28-026, FS28-091, FS28-185 and FS28-256 (all in IgG1 LALA format) showed dose-dependent binding to NCI-H226 cells. FS28-024, FS28-026 and FS28-091 showed high affinity binding to cell surface MSLN with low nanomolar EC₅₀ values, similar to the positive control anti-MSLN mAb SS1. mAbs FS28-185 and FS28-256 showed weaker binding to cell surface MSLN with EC₅₀ values over 30 nM and lower Eₘₐₓ values compared with FS28-024, FS28-026 and FS28-091.

FIG. 2 shows the ADCC activity of anti-MSLN mAb². The anti-human MSLN mAb² FS28-024-052 and FS28-256-271, as well as the positive control antibody SS1, were tested in hIgG1 LALA format (no effector function) and effector competent backbone hIgG1. FS28-024-052 had the highest ADCC activity of the tested antibodies, with FS28-256-271 having comparable ADCC activity to the control antibody. In all cases, introduction of the LALA mutations into the IgG1 backbone completely abrogated any ADCC activity, as expected.

FIG. 3 shows individual tumour volume measurements in the CT26.G10 syngeneic tumour model treated with (A) G1-AA/HeID1.3 (human IgG1 control), (B) FS22m-063-AA/HeID1.3 (anti-mouse CD137 Fcab in mAb² format), (C) combination of FS22m-063-AA/HeID1.3 and G1-AA/FS28m-228-010 (anti-mouse CD137 Fcab plus anti-mouse MSLN Fab), (D) FS22m-063-AA/FS28m-228-010 (anti-mouse CD137/MSLN mAb²) (E) FS22m-063-AA/4420 (anti-mouse CD137 Fcab in mAb² format), and (F) G1-AA/FS28m-228-010 (anti-mouse MSLN Fab). FS22m-063-AA/FS28m-228-010 showed reduced tumour growth compared to the isotype control, as well as other treatment groups.

FIG. 4 shows a Kaplan Meier survival curve for the CT26.G10 syngeneic tumour model treated with G1-AA/HeID1.3 (IgG control), FS22m-063-AA/HeID1.3 (anti-mouse CD137 Fcab in mAb² format), FS22m-063-AA/4420 (anti-mouse CD137 Fcab in mAb² format), G1-AA/FS28m-228-010 (anti-mouse MSLN Fab), combination of FS22m-063-AA/HeID1.3 and G1-AA/FS28m-228-010 (anti-mouse CD137 Fcab plus anti-mouse MSLN Fab), and FS22m-063-AA/FS28m-228-010 (anti-mouse CD137/MSLN mAb²). The results show that FS22m-063-AA/FS28m-228-010 treatment resulted in significantly improved survival compared to mice treated with the isotype control or other treatment groups.

### DETAILED DESCRIPTION

The present invention relates to antibody molecules that bind MSLN. The antibody molecule preferably binds human MSLN, more preferably human and cynomolgus MSLN. The antibody molecule of the present invention is preferably capable of binding to MSLN expressed on the surface of a cell. The cell is preferably a tumour cell.

The antibody molecule preferably binds MSLN specifically. The term “specific” may refer to the situation in which the antibody molecule will not show any significant binding to molecules other than its specific binding partner(s), here MSLN. The term “specific” is also applicable where the antibody molecule is specific for particular epitopes, such as epitopes on MSLN, that are carried by a number of antigens, in which case the antibody molecule will be able to bind to the various antigens carrying the epitope. The antibody molecule preferably does not bind, or does not show any significant binding, to other molecules involved in cell adhesion, such as CEACAM-5, E-Cadherin, thrombomodulin and/or EpCAM.

As explained in the background section above, mature MSLN is shed from tumour cells and is cleared from the tumour site. This shed MSLN can act as a sink for anti-MSLN binding molecules which after binding to the shed MSLN are also cleared from the tumour site. In order to select for molecules which preferentially bind to MSLN present on the surface of tumour cells, the present inventors selected for antibody molecules with high avidity for MSLN. Specifically, the present inventors selected antibody molecules which bound to immobilised MSLN with higher affinity than to MSLN in solution. Antibody molecules which bind to MSLN with high avidity are thought to preferentially bind to MSLN present on tumour cells where multiple copies of MSLN are expected to be present and available for bivalent binding by the antibody molecule, as opposed to mesothelin shed from the tumour cells which is expected to be in monomeric form. Without wishing to be bound by theory, the antibody molecules of the invention are therefore expected to be cleared from the tumour site less quickly, and hence to have a longer time window in which to exert their therapeutic effect.

The antibody molecule preferably binds to immobilised MSLN with a higher affinity than to MSLN in solution. Immobilised MSLN may be MSLN immobilised at a surface, such as chip for use in surface plasmon resonance. MSLN in solution is also referred to as soluble MSLN herein and is not immobilised. The soluble MSLN is preferably in monomeric form, i.e. monomeric mesothelin.

The affinity of an antibody for its cognate antigen can be expressed as the equilibrium dissociation constant (K_{D}) with which the antibody binds said antigen. The higher the K_{D} value, the lower the affinity of the antibody molecule for the antigen.

The antibody molecule preferably binds to immobilised MSLN with an affinity (K_{D}) of at least 9 nM, 8 nM, 7 nM, or 6 nM or with a higher affinity. Preferably, the antibody molecule binds to immobilised MSLN with an affinity (K_{D}) of at least 7 nM, 6 nM or with a higher affinity.

The antibody molecule preferably binds to immobilised MSLN with an affinity (K_{D}) of 15 nM or with an affinity that is lower. More preferably, the antibody molecule binds to immobilised MSLN with an affinity (K_{D}) of 16 nM, 17 nM, or 18 nM, or with an affinity that is lower.

In a preferred embodiment, the antibody molecule binds immobilised MSLN with an affinity (K_{D}) of 6 nM or with a higher affinity, and binds MSLN in solution with an affinity (K_{D}) of 18 nM or with a lower affinity.

The binding affinity of an antibody molecule for cells comprising surface-bound MSLN may be measured by determining the concentration of the antibody molecule needed to achieve half-maximal binding (EC₅₀) of the antibody molecule to the cells. Suitable methods for determining the concentration of an antibody molecule needed to achieve half-maximal binding of an antibody molecule to cells are known in the art and disclosed in the present Examples (see e.g. Example 4). As explained above, antibody molecules whose binding to tumour cells comprising surface-bound MSLN is not affected or less affected by the presence of soluble mesothelin are preferred in view of the presence of shed MSLN in the tumour environment. Thus, in a preferred embodiment, the concentration of the antibody molecule needed to achieve half-maximal binding (EC₅₀) of the antibody to cells (e.g. tumour cells) comprising surface-bound MSLN in the presence of soluble MSLN is less than 20-fold, less than 15-fold, less than 10-fold, less than 9-fold, less than 8-fold, less than 7-fold, less than 6-fold, less than 5-fold, less than 4-fold, or less than 3-fold higher than the concentration of the antibody molecule needed to achieve half-maximal binding (EC₅₀) of the antibody to the cells in the absence of soluble MSLN.

The binding of antibody molecules, which do not block binding of MUC16 to MSLN, to cells comprising cell-bound MSLN has been shown to be less affected by the presence of soluble MSLN. Thus, an antibody molecule which is not capable of, or does not block, binding of MUC16 to MSLN may be preferred.

Alternatively, the antibody molecule may block binding of MUC16 to MSLN.

The immobilised MSLN may have the sequence set forth in SEQ ID NO: 169. The MSLN in solution may have the sequence set forth in SEQ ID NO: 169.

The antibody molecules of the invention have also been shown to bind cynomolgus MSLN. Binding to cynomolgus MSLN as well as human MSLN is thought to be beneficial for carrying out efficacy and toxicity studies with the antibody molecule in cynomolgus monkeys, which may be predictive of the efficacy and toxicity of the antibody molecule in humans.

The antibody molecule may bind to immobilised human MSLN and immobilised cynomolgus MSLN with similar affinity. In addition, the antibody molecule may bind to human MSLN in solution and cynomolgus MSLN in solution with similar affinity. This is thought to be beneficial for ensuring that efficacy and toxicity studies carried out with the antibody molecule in cynomolgus monkeys are predictive of the efficacy and toxicity of the antibody molecule in humans.

Thus, in a preferred embodiment, the antibody molecule binds to immobilised cynomolgus MSLN with an affinity which is no more than 10-fold, preferably no more than 5-fold, more preferably no more than 2-fold lower or higher than the affinity with which the antibody molecule binds immobilised human MSLN. In addition, the antibody molecule preferably binds to cynomolgus MSLN in solution with an affinity which is no more than 10-fold, preferably no more than 5-fold, more preferably no more than 2-fold lower or higher than the affinity with which the antibody molecule binds human MSLN in solution.

The binding affinity of an antibody molecule to a cognate antigen, such as human or cynomolgus MSLN can be determined by surface plasmon resonance (SPR), such as Biacore, for example.

The antibody molecule may be chimeric, humanised or human. Preferably, the antibody molecule is a human antibody molecule.

The antibody molecule is preferably monoclonal.

The antibody molecule may be isolated, in the sense of being free from contaminants, such as antibodies able to bind other polypeptides and/or serum components.

The antibody molecule may be natural or partly or wholly synthetically produced. For example, the antibody molecule may be a recombinant antibody molecule.

The antibody molecule comprises one or more CDR-based antigen-binding sites for MSLN.

The antibody molecule may be an immunoglobulin or an antigen-binding fragment thereof.

For example, the antibody molecule may be an IgG, IgA, IgE or IgM molecule, preferably an IgG molecule, such as an IgG1, IgG2, IgG3 or IgG4 molecule, more preferably an IgG1 or IgG2 molecule, most preferably an IgG1 molecule, or a fragment thereof. In a preferred embodiment, the antibody molecule is a complete immunoglobulin molecule.

In other embodiments, the antibody molecule may be an antigen-binding fragment comprising a CDR-based antigen-binding site for MSLN. For example, the antigen-binding fragment may be a fragment antigen-binding (Fab), IgGΔCH2, F(ab′)₂, single-chain Fab (scFab), a disulphide stabilized variable fragment (dsFv), a single-chain variable fragment (scFv), (scFv)₂, an scFv-CH3 (minibody), scFv-Fc, scFv-zipper, a diabody, a triabody, a tetrabody, or a single-domain antibody (sdAb), such as a V_{H}H domain or nanobody. Preferred antigen-binding fragments comprise more than one CDR-based antigen-binding site for MSLN, i.e. they may be multivalent. Thus, the antigen-binding fragment may preferably be an IgGΔCH2, F(ab′)₂, a diabody, a triabody, or a tetrabody. (Brinkmann and Kontermann, (2017) and Powers et al, (2012))

Antibodies and methods for their construction and use are well-known in the art and are described in, for example, Holliger and Hudson, 2005. It is possible to take monoclonal and other antibodies and use techniques of recombinant DNA technology to produce other antibodies or chimeric molecules which retain the specificity of the original antibody. Such techniques may involve introducing CDRs or variable regions of one antibody molecule into a different antibody molecule (EP-A-184187, GB 2188638A and EP-A-239400).

A CDR-based antigen-binding site is an antigen-binding site in an antibody variable region. A CDR-based antigen-binding site, may be formed by three CDRs, such as the three light chain variable domain (VL) CDRs or three heavy chain variable domain (VH) CDRs. Preferably the CDR-based antigen-binding site is formed by six CDRs, three VL CDRs and three VH CDRs. The contributions of the different CDRs to the binding of the antigen may vary in different antigen binding sites.

The three VH domain CDRs of the antigen-binding site may be located within an immunoglobulin VH domain and the three VL domain CDRs may be located within an immunoglobulin VL domain. For example, the CDR-based antigen-binding site may be located in an antibody variable region.

The antibody molecule has one or preferably more than one, for example two, CDR-based antigen binding sites for MSLN. The antibody molecule thus may comprise one VH and one VL domain but preferably comprises two VH and two VL domains, i.e. two VH/VL domain pairs, as is the case in naturally-occurring IgG molecules, for example.

The CDR-based antigen-binding site may comprise the three VH CDRs or three VL CDRs, preferably the three VH CDRs and the three VL CDRs, of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, or FS28-024, preferably antibody FS28-256-271, or FS28-024-052, most preferably antibody FS28-256-271.

The sequences of the CDRs may be readily determined from the VH and VL domain sequences of an antibody molecule using routine techniques. The VH and VL domain sequences of antibodies FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024, are described herein, and the three VH and three VL domain CDRs of said antibodies may thus be determined from said sequences. The CDR sequences may, for example, be determined according to Kabat et al., 1991 or the international ImMunoGeneTics information system (IMGT) (Lefranc et al., 2015).

The VH domain CDR1, CDR2 and CDR3 sequences of the antibody molecule according to IMGT numbering may be the sequences located at positions 27-38, 56-65, and 105-117, of the VH domain of the antibody molecule, respectively.

The VH domain CDR1, CDR2 and CDR3 sequences of the antibody molecule according to Kabat numbering may be the sequences at located positions 31-35, 50-65, and 95-102 of the VH domain, respectively.

The VL domain CDR1, CDR2 and CDR3 sequences of the antibody molecule according to IMGT numbering may be the sequences located at positions 27-38, 56-65, and 105-117, of the VL domain, respectively.

The VL domain CDR1, CDR2 and CDR3 sequences of the antibody molecule according to Kabat numbering may be the sequences at located positions 24-34, 50-56, and 89-97 of the VL domain, respectively.

For example, the sequence of the VH domain CDR1, CDR2 and CDR3 of:
(i) FS28-256-271 may be as set forth in SEQ ID NO: 98, 73, and 99, respectively;
(ii) FS28-024-052 may be as set forth in SEQ ID NO: 10, 11, and 41, respectively;
(iii) FS28-256-021 may be as set forth in SEQ ID NO: 98, 73 and 99, respectively;
(iv) FS28-256-012 may be as set forth in SEQ ID NO: 98, 73 and 99, respectively;
(v) FS28-256-023 may be as set forth in SEQ ID NO: 101, 73 and 103, respectively;
(vi) FS28-256-024 may be as set forth in SEQ ID NO: 98, 73 and 99, respectively;
(vii) FS28-256-026 may be as set forth in SEQ ID NO: 101, 73 and 103, respectively;
(viii) FS28-256-027 may be as set forth in SEQ ID NO: 98, 73 and 99, respectively;
(ix) FS28-256-001 may be as set forth in SEQ ID NO: 85, 73 and 75, respectively;
(x) FS28-256-005 may be as set forth in SEQ ID NO: 85, 73 and 75, respectively;
(xi) FS28-256-014 may be as set forth in SEQ ID NO: 111, 73 and 113, respectively;
(xii) FS28-256-018 may be as set forth in SEQ ID NO: 101, 73 and 103, respectively;
(xiii) FS28-256 may be as set forth in SEQ ID NO: 71, 73 and 75, respectively;
(xiv) FS28-024-051 may be as set forth in SEQ ID NO: 10, 11 and 32, respectively;
(xv) FS28-024-053 may be as set forth in SEQ ID NO: 10, 11 and 51, respectively; and
(xvi) FS28-024 may be as set forth in SEQ ID NO: 10, 11 and 12, respectively;
   wherein the CDR sequences are defined according to the IMGT numbering scheme.

The sequence of the VL domain CDR1, CDR2 and CDR3 of:
(i) FS28-256-271 may be as set forth in SEQ ID NO: 20, 21, and 44, respectively;
(ii) FS28-024-052 may be as set forth in SEQ ID NO: 20, 21 and 22, respectively;
(iii) FS28-256-021 may be as set forth in SEQ ID NO: 20, 21 and 34, respectively;
(iv) FS28-256-012 may be as set forth in SEQ ID NO: 20, 21 and 25, respectively;
(v) FS28-256-023 may be as set forth in SEQ ID NO: 20, 21 and 34, respectively;
(vi) FS28-256-024 may be as set forth in SEQ ID NO: 20, 21 and 43, respectively;
(vii) FS28-256-026 may be as set forth in SEQ ID NO: 20, 21 and 43, respectively;
(viii) FS28-256-027 may be as set forth in SEQ ID NO: 20, 21 and 44, respectively;
(ix) FS28-256-001 may be as set forth in SEQ ID NO: 20, 21 and 34, respectively;
(x) FS28-256-005 may be as set forth in SEQ ID NO: 20, 21 and 43, respectively;
(xi) FS28-256-014 may be as set forth in SEQ ID NO: 20, 21 and 25, respectively;
(xii) FS28-256-018 may be as set forth in SEQ ID NO: 20, 21 and 25, respectively;
(xiii) FS28-256 may be as set forth in SEQ ID NO: 20, 21 and 25, respectively;
(xiv) FS28-024-051 may be as set forth in SEQ ID NO: 20, 21 and 22, respectively;
(xv) FS28-024-053 may be as set forth in SEQ ID NO: 20, 21 and 22, respectively; and
(xvi) FS28-024 may be as set forth in SEQ ID NO: 20, 21 and 22, respectively;
   wherein the CDR sequences are defined according to the IMGT numbering scheme.

For example, the sequence of the VH domain CDR1, CDR2 and CDR3 of:
(i) FS28-256-271 may be as set forth in SEQ ID NO: 97,182, and 100, respectively;
(ii) FS28-024-052 may be as set forth in SEQ ID NO: 13, 14 and 42, respectively;
(iii) FS28-256-021 may be as set forth in SEQ ID NO: 97, 74 and 100, respectively;
(iv) FS28-256-012 may be as set forth in SEQ ID NO: 97, 74 and 100, respectively;
(v) FS28-256-023 may be as set forth in SEQ ID NO: 102, 74 and 104, respectively;
(vi) FS28-256-024 may be as set forth in SEQ ID NO: 97, 74 and 100, respectively;
(vii) FS28-256-026 may be as set forth in SEQ ID NO: 102, 74 and 104, respectively;
(viii) FS28-256-027 may be as set forth in SEQ ID NO: 97, 74 and 100, respectively;
(ix) FS28-256-001 may be as set forth in SEQ ID NO: 86, 74 and 76, respectively;
(x) FS28-256-005 may be as set forth in SEQ ID NO: 86, 74 and 76, respectively;
(xi) FS28-256-014 may be as set forth in SEQ ID NO: 112, 74 and 114, respectively;
(xii) FS28-256-018 may be as set forth in SEQ ID NO: 102, 74 and 104, respectively;
(xiii) FS28-256 may be as set forth in SEQ ID NO: 72, 74 and 76, respectively;
(xiv) FS28-024-051 may be as set forth in SEQ ID NO: 13, 14 and 33, respectively;
(xv) FS28-024-053 may be as set forth in SEQ ID NO: 13, 14 and 52, respectively; and
(xvi) FS28-024 may be as set forth in SEQ ID NO: 13, 14 and 15, respectively;
   wherein the CDR sequences are defined according to the Kabat numbering scheme.

The sequence of the VL domain CDR1, CDR2 and CDR3 of:
(i) FS28-256-271 may be as set forth in SEQ ID NO: 23, 24, and 44, respectively;
(ii) FS28-024-052 may be as set forth in SEQ ID NO: 23, 24 and 22, respectively;
(iii) FS28-256-021 may be as set forth in SEQ ID NO: 23, 24 and 34, respectively;
(iv) FS28-256-012 may be as set forth in SEQ ID NO: 23, 24 and 25, respectively;
(v) FS28-256-023 may be as set forth in SEQ ID NO: 23, 24 and 34, respectively;
(vi) FS28-256-024 may be as set forth in SEQ ID NO: 23, 24 and 43, respectively;
(vii) FS28-256-026 may be as set forth in SEQ ID NO: 23, 24 and 43, respectively;
(viii) FS28-256-027 may be as set forth in SEQ ID NO: 23, 24 and 44, respectively;
(ix) FS28-256-001 may be as set forth in SEQ ID NO: 23, 24 and 34, respectively;
(x) FS28-256-005 may be as set forth in SEQ ID NO: 23, 24 and 43, respectively;
(xi) FS28-256-014 may be as set forth in SEQ ID NO: 23, 24 and 25, respectively;
(xii) FS28-256-018 may be as set forth in SEQ ID NO: 23, 24 and 25, respectively;
(xiii) FS28-256 may be as set forth in SEQ ID NO: 23, 24 and 25, respectively;
(xiv) FS28-024-051 may be as set forth in SEQ ID NO: 23, 24 and 22, respectively;
(xv) FS28-024-053 may be as set forth in SEQ ID NO: 23, 24 and 22, respectively; and
(xvi) FS28-024 may be as set forth in SEQ ID NO: 23, 24 and 22, respectively;
   wherein the CDR sequences are defined according to the Kabat numbering scheme.

The CDR-based antigen-binding site may comprise the VH or VL domains, preferably the VH and VL domains, of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, or FS28-024, preferably antibody FS28-256-271, or FS28-024-052, most preferably antibody FS28-256-271.

The VH domain of antibodies FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024, may have the sequence set forth in SEQ ID NO: 180, 39, 109, 109, 121, 109, 121, 109, 63, 63, 115, 121, 69, 30, 49, and 8 respectively.

The VL domain of antibodies FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024 may have the sequence set forth in SEQ ID NO: 56, 18, 93, 79, 93, 53, 53, 56, 93, 53, 79, 79, 79, 18, 18, and 18, respectively.

The antibody molecule may comprise the heavy or light chain, preferably the heavy and light chain, of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, or FS28-024, preferably antibody FS28-256-271, or FS28-024-052, most preferably antibody FS28-256-271.

The heavy chain (with LALA mutation) of antibodies FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024, may have the sequence set forth in SEQ ID NO: 178, 37, 107, 107, 125, 107, 125, 107, 89, 89, 119, 125, 67, 28, 47, 5, respectively.

The heavy chain (without LALA mutation) of antibodies FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024, may have the sequence set forth in SEQ ID NO: 176, 35, 105, 105, 123, 105, 123, 105, 81, 87, 117, 123, 65, 26, 45, and 4, respectively.

The light chain of antibodies FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024, may have the sequence set forth in SEQ ID NO: 95, 16, 83, 77, 83, 90, 90, 95, 83, 90, 77, 77, 77, 16, 16, and 16, respectively.

The antibody molecule may also comprise a variant of a CDR, VH domain, VL domain, heavy chain or light chain sequence as described herein. Suitable variants can be obtained by means of methods of sequence alteration, or mutation, and screening. In a preferred embodiment, an antibody molecule comprising one or more such variant sequences retains one or more of the functional characteristics of the parent antibody molecule, such as binding specificity and/or binding affinity for MSLN, preferably human and/or cynomolgus MSLN. For example, an antibody molecule comprising one or more variant sequences preferably binds to MSLN with the same affinity as, or a higher affinity than, the (parent) antibody molecule. The parent antibody molecule is antibody molecule which does not comprise the amino acid substitution(s), deletion(s), and/or insertion(s) which has (have) been incorporated into the variant antibody molecule.

An antibody molecule which comprises CDRs 1-6, the VH domain, and/or the heavy chain of antibody FS28-256-021, FS28-256-012, FS28-256-023, FS28-256-024, FS28-256-026, FS28-256-027, FS28-256-001, FS28-256-005, FS28-256-014, FS28-256-018, or FS28-256, may comprise an amino acid substitution at position 55 or 57 of the VH domain, wherein the amino acid residue numbering is according to the IMGT numbering scheme.

For example, the antibody molecule may comprise CDRs 1-6, the VH domain, and/or the heavy chain of antibody FS28-256-027, wherein the antibody molecule comprises an amino acid substitution at position 55 of the VH domain, wherein the amino acid residue numbering is according to the IMGT numbering scheme.

The antibody molecule of the invention may comprise a VH domain, VL domain, heavy chain, or light chain, which has at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the VH domain, VL domain, heavy chain, or light chain of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, or FS28-024, preferably antibody FS28-256-271, or FS28-024-052, most preferably antibody FS28-256-271.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equaling 12 and a gap extension penalty equaling 4. Use of GAP may be preferred but other algorithms may be used, e.g. BLAST (which uses the method of Altschul et al., 1990, FASTA (which uses the method of Pearson and Lipman, 1988), or the Smith-Waterman algorithm (Smith and Waterman, 1981), or the TBLASTN program, of Altschul et al., 1990 supra, generally employing default parameters. In particular, the psi-Blast algorithm (Altschul et al., 1997) may be used.

The antibody molecule may comprise a VH CDR1, VH CDR2, VH CDR3, VL CDR1, VL CDR2, and/or VL CDR3 which has one or more amino acid sequence alterations (addition, deletion, substitution and/or insertion of an amino acid residue), preferably 3 alterations or fewer, 2 alterations or fewer, or 1 alteration compared with the VH CDR1, VH CDR2, VH CDR3, VL CDR1, VL CDR2, and/or VL CDR3 of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, or FS28-024, preferably antibody FS28-256-271, or FS28-024-052, most preferably antibody FS28-256-271.

The antibody molecule may comprise a VH domain, VL domain, heavy chain, or light chain, which has one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with the VH domain, VL domain, heavy chain, or light chain of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, or FS28-024, preferably antibody FS28-256-271, or FS28-024-052, most preferably antibody FS28-256-271. In particular, amino acid sequence alterations may be located in one or more framework regions of the antibody molecules, such as one or more framework regions of the heavy and/or light chains of the antibody molecule.

In preferred embodiments in which one or more amino acids are substituted with another amino acid, the substitutions may conservative substitutions, for example according to the following Table. In some embodiments, amino acids in the same category in the middle column are substituted for one another, i.e. a non-polar amino acid is substituted with another non-polar amino acid, for example. In some embodiments, amino acids in the same line in the rightmost column are substituted for one another.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g. binding affinity) of the antibody molecule comprising the substitution as compared to the equivalent unsubstituted antibody molecule.

The heavy chain of the antibody molecule may optionally comprise an additional lysine residue (K) at the immediate C-terminus of the heavy chain CH3 domain sequence.

The CH2 domain is known bind to Fcγ receptors and complement. Binding of the CH2 domain to Fcγ receptors is required antibody-dependent cell-mediated cytotoxicity (ADCC), while binding to complement is required complement-dependent cytotoxicity (CDC). Where the antibody molecule is used in the treatment of cancer and is not in the form of an ADC, ADCC activity is expected to lead to killing of the cancer cells and should therefore preferably be retained. However, where the antibody molecule is in the form of an ADC and is conjugated to a bioactive molecule, mutations to reduce or abrogate ADCC activity are expected to be beneficial to avoid killing of the target cancer cells via ADCC before the bioactive molecule is delivered to the cell. In addition, mutations to reduce or abrogate ADCC and/or CDC activity is expected to be useful where the antibody molecule comprises a second antigen-binding site for an immune cell antigen as described herein, where ADCC and/or CDC-mediated killing of immune cells bound by the antibody molecule should be avoided.

The CH2 domain of the antibody molecule may thus comprise one or more mutations that reduce or abrogate binding of the CH2 domain to one or more Fcγ receptors, such as FcγRI, FcγRIIa, FcγRIIb, FcγRIII, and/or to complement. The inventors postulate that reducing or abrogating binding to Fcγ receptors will decrease or eliminate ADCC mediated by the antibody molecule. Similarly, reducing or abrogating binding to complement is expected to reduce or eliminate CDC mediated by the antibody molecule. Mutations to decrease or abrogate binding of the CH2 domain to one or more Fcγ receptors and/or complement are known in the art (Wang et al., 2018). These mutations include the “LALA mutation” described in Bruhns et al., 2009 and Hezareh et al., 2001, which involves substitution of the leucine residues at positions 1.3 and 1.2 of the CH2 domain with alanine (L1.3A and L1.2A). Alternatively, the generation of a-glycosyl antibodies through mutation of the conserved N-linked glycosylation site by mutating the aparagine (N) at position 84.4 of the CH2 domain to alanine, glycine or glutamine (N84.4A, N84.4G or N84.4Q) is also known to decrease IgG1 effector function (Wang et al., 2018). As a further alternative, complement activation (Clq binding) and ADCC are known to be reduced through mutation of the proline at position 114 of the CH2 domain to alanine or glycine (P114A or P114G) (Idusogie et al., 2000; Klein et al., 2016). These mutations may also be combined in order to generate antibody molecules with further reduced or no ADCC or CDC activity.

Thus, the antibody molecule may comprise a CH2 domain, wherein the CH2 domain preferably comprises:
(i) alanine residues at positions 1.3 and 1.2; and/or
(ii) an alanine or glycine at position 114; and/or
(iii) an alanine, glutamine or glycine at position 84.4;
wherein the amino acid residue numbering is according to the IMGT numbering scheme.

In a preferred embodiment, the antibody molecule comprises a CH2 domain, wherein the CH2 domain comprises:
(i) an alanine residue at position 1.3; and
(ii) an alanine residue at position 1.2;
wherein the amino acid residue numbering is according to the IMGT numbering scheme.

For example, the CH2 domain may comprise a LALA mutation and have the sequence set forth in SEQ ID NO: 173.

In an alternative preferred embodiment, the antibody molecule comprises a CH2 domain, wherein the CH2 domain comprises:
(i) an alanine residue at position 1.3;
(ii) an alanine residue at position 1.2; and
(iii) an alanine at position 114;
wherein the amino acid residue numbering is according to the IMGT numbering scheme.

For example, the CH2 domain may comprise a LALA-PA mutation and have the sequence set forth in SEQ ID NO: 174.

Also contemplated is antibody molecule which comprises a CDR-based antigen binding site for MSLN and which competes with an antibody molecule as described herein, or that binds to the same epitope on MSLN as an antibody molecule as described herein. Methods for determining competition for an antigen by two antibodies are known in the art. For example, competition of binding to an antigen by two antibodies can be determined by surface plasmon resonance, e.g. using a Biacore instrument. Methods for mapping the epitope bound by an antibody are similarly known.

The N-terminal region of MSLN has been reported to interact with MUC16. It has been reported that this interaction may play a role in cancer cell adhesion.

The antibody molecules have been shown to have range of activities on ligand binding. For example, the antibody molecule may be capable of blocking, or may not be capable of blocking binding of MUC16 to MSLN. Alternatively, the antibody molecule may be capable of enhancing binding of MUC16 to MSLN. For example, the antibody molecule may comprise CDRs 1-6, the VH domain, VL domain, heavy chain and/or light chain of antibody FS28-024-051, FS28-024-052, FS28-024-053, or FS28-024, or a variant thereof, wherein the antibody molecule blocks binding of MUC16 to MSLN.

In a preferred embodiment, the antibody molecule does not block binding of MUC16 to MSLN. For example, the antibody molecule may comprise CDRs 1-6, the VH domain, VL domain, heavy chain and/or light chain of antibody FS28-256-271, FS28-256-021, FS28-256-012, FS28-256-023, FS28-256-024, FS28-256-026, FS28-256-027, FS28-256-001, FS28-256-005, FS28-256-014, FS28-256-018, FS28-256, or a variant thereof, wherein the antibody molecule does not block binding of MUC16 to MSLN.

Methods which are suitable for determining the ability of an antibody molecule to block the binding of MUC16 to MSLN are known in the art and include ELISAs and cell-based assays, for example an assay where the antibody competes for binding with MUC16 for binding to cells expressing MSLN, such as NCI-H226 cells

In a preferred embodiment, the antibody molecule may comprise one or more further antigen-binding sites that bind one or more further antigens, in addition to the CDR-based antigen binding site for MSLN. The one or more further antigen-binding sites preferably bind their cognate antigens specifically.

The one or more further antigen-binding sites preferably do not bind MSLN. The antibody molecule may thus be a multispecific, for example a bispecific, trispecific, or tetraspecific molecule, preferably a bispecific molecule. In a preferred embodiment, the antibody molecule is capable of simultaneously binding to MSLN and the one or more further antigens.

Antibody molecules are known to have a modular architecture comprising discrete domains, which can be combined in a multitude of different ways to create multispecific, e.g. bispecific, trispecific, or tetraspecific antibody formats. Exemplary multispecific antibody formats are described in Spiess et al. (2015) and Kontermann (2012), for example. The antibody molecules of the present invention may be employed in such multispecific formats.

For example, the antibody molecule of the invention may be a heterodimeric antibody molecule, such as a heterodimeric complete immunoglobulin molecule, or a fragment thereof. In this case, one part of the antibody molecule will have a sequence or sequences as described herein. For example, where the antibody molecule of the invention is a bispecific heterodimeric antibody molecule, the antibody molecule may comprise a heavy chain and light chain as described herein paired with a heavy chain and light chain comprising a VH domain and a VL domain, respectively, which bind an antigen other than MSLN or additionally binds to another epitope on MSLN. Techniques for preparing heterodimeric antibodies are known in the art and include knobs-into-holes (KIHs) technology, which involves engineering the CH3 domains of an antibody molecule to create either a “knob” or a “hole” to promote chain heterodimerization. Alternatively, heterodimeric antibodies can be prepared through the introduction of charge pairs into the antibody molecule to avoid homodimerization of CH3 domains by electrostatic repulsion and to direct heterodimerization by electrostatic attraction. Examples of heterodimeric antibody formats include CrossMab, mAb-Fv, SEED-body, and kih IgG.

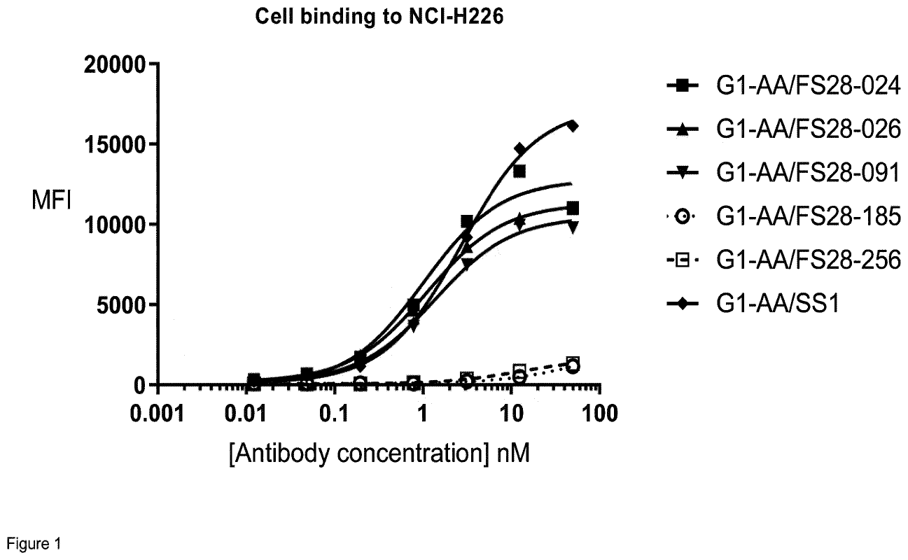

Alternatively, a multispecific antibody molecule may comprise a complete immunoglobulin molecule or a fragment thereof and an additional antigen-binding moiety or moieties. The antigen-binding moiety may for example be an Fv, scFv or single domain antibody, and may be fused to the complete immunoglobulin molecule or a fragment thereof. Examples of multispecific antibody molecules comprising additional antigen-binding moieties fused to a complete immunoglobulin molecule include DVD-IgG, DVI-IgG, scFv4-IgG, IgG-scFv, and scFv-IgG molecules (Spiess et al., 2015; FIG. 1). Examples of multispecific antibody molecules comprising additional antigen-binding moieties fused to an immunoglobulin fragment include BiTE molecules, diabodies, and DART molecules, for example (Spiess et al., 2015; FIG. 1). Other suitable formats would be readily apparent to the skilled person.

In a preferred embodiment, the antibody molecule comprises a second antigen-binding site that binds a second antigen, wherein the second antigen-binding site is located in a constant domain of the antibody molecule. For example, the antibody molecule may be a mAb²™ bispecific antibody. A mAb² bispecific antibody, as referred to herein, is an IgG immunoglobulin which includes a CDR-based antigen binding site in each of its variable regions and at least one antigen binding site in a constant domain of the antibody molecule.

In a preferred embodiment, the antibody is an antibody molecule that binds MSLN and a second antigen, the antibody molecule comprising:
(i) two CDR-based antigen-binding sites for MSLN, each formed by an immunoglobulin VH domain and an immunoglobulin VL domain; and
(ii) two antigen-binding sites that bind a second antigen located in the two CH3 domains of the antibody molecule.

In a more preferred embodiment, the antibody is a complete immunoglobulin molecule, e.g. a complete IgG1 molecule that binds MSLN and a second antigen, the antibody molecule comprising:
(i) two CDR-based antigen-binding sites for MSLN, each formed by an immunoglobulin VH domain and an immunoglobulin VL domain; and
(ii) two antigen-binding sites that bind a second antigen located in the two CH3 domains of the antibody molecule; and
wherein the immunoglobulin molecule further comprises CH1, CH2 and CL domains.

The antigen-binding site for the second antigen may be located in any constant domain of the antibody molecule. For example, the antigen-binding site for the second antigen may be located in one or more of the CH4, CH3, CH2, CH1 or CL domains, preferably the CH3 or CH2 domain, most preferably the CH3 domain.

The antigen binding site may be composed of one or more, for example one, two, three or more, structural loops of the constant domain of the antibody molecule.

The structural loops of an antibody constant domain include the AB, BC, CD, DE, EF, and FG structural loops. The antigen binding site may comprise two or more of the AB, BC, CD, DE, EF, and FG structural loops of the constant domain, preferably the AB and EF structural loops, or the AB, CD and EF structural loops.

The positions of the structural loops in antibody constant domains are well-known in the art. For example, the structural loops of the CH3 domain are located between positions 10 and 19 (AB loop), 28 and 39 (BC loop), 42 and 79 (CD loop), 82 and 85 (DE loop), 91 and 102 (EF loop) and 106 and 117 (FG loop) of the CH3 domain, wherein the residues are numbered according to IMGT numbering scheme. The locations of the structural loop positions in other constant domains may be easily determined.

The structural loops of the constant domain may comprise one or more amino acid modifications in order to form the antigen-binding site for the second antigen. One or more amino acid modifications may include amino acid substitutions, additions, or deletions. The introduction of amino acid modifications into the structural loop regions of antibody constant domains to create antigen-binding sites for target antigens is well-known in the art and is described, for example, in Wozniak-Knopp G et al., 2010, WO2006/072620 and WO2009/132876. Examples of constant domain binding sites are provided below.

In a preferred embodiment, the antibody molecule comprises one or more amino acid modifications (substitutions, additions, and/or deletions) in the AB, CD and/or EF structural loops, preferably the AB and EF structural loops or the AB, CD and EF structural loops. For example, the antibody molecule may comprise one or more amino acid modifications (substitutions, additions, and/or deletions) at positions 11-18, 43-78 and/or 92-101 of the CH3 domain, preferably at positions 11-18 and 92-101, or at positions 11-18, 43-78 and 92-101 of the CH3 domain to provide an antigen-binding site for a second antigen as set out herein. More preferably, the antibody molecule comprises one or more amino acid modifications (substitutions, additions, and/or deletions) at positions 12-18, 45.1 to 78, 92 to 94, and/or 97-98 of the CH3 domain, more preferably at positions 12-18, 92 to 94, and 97-98, or at positions 12-18, 45.1 to 78, 92 to 94, and 97-98 of the CH3 domain to provide an antigen-binding site for a second antigen as set out herein. The unmodified CH3 domain preferably comprises or consists of the sequence set forth in SEQ ID NO: 172. The residue numbering is according to IMGT numbering scheme.

The second antigen bound by the second antigen-binding site of the antibody molecule may be an immune cell antigen, preferably a member of the tumour necrosis factor receptor superfamily (TNFRSF). TNFRSF receptors are membrane-bound cytokine receptors that comprise an extracellular cysteine rich domain that binds one or more ligands of the tumour necrosis factor superfamily (TNFSF).

The TNFRSF receptor is preferably located on the surface of an immune cell, such as a T cell, an antigen presenting cell (APC), an NK cell and/or a B cell, preferably a T cell. Upon binding of a TNFRSF ligand, TNFRSF receptors form clusters on the immune cell surface which activates the immune cell. For example, ligand bound TNFRSF receptors may form multimers, such as trimers, or clusters of multimers. The presence of clusters of ligand-bound TNFRSF receptors stimulates intracellular signalling pathways which activate the immune cell.

Without wishing to be bound by theory it is thought that by engaging both MSLN on a tumour cell surface and a TNFRSF receptor on an immune cell surface, the antibody molecules will be crosslinked through binding to MSLN and thereby drive clustering and activation of the TNFRSF receptor and hence activation of the immune cell(s). In other words, the antibody molecule will act as a TNFRSF receptor agonist when both targets are bound. The activated immune cells may then then initiate, promote or take part in an immune response against the cancer expressing cell-surface bound MSLN. An overview of the role the immune system plays in recognizing and eradicating cancer cells is provided by Chen and Mellman (2013).

Antibody molecule can be crosslinked through binding to Fcγ receptors but this is both inefficient and cannot be targeted to a particular location e.g. the site of a disease, as Fcγ receptor expressing cells are present throughout the human body. In a preferred embodiment, an antibody molecule comprising a second antigen-binding site for a TNFRSF receptor therefore comprises one or mutation to reduce or abrogate binding to one or more Fcγ receptors as described herein.

The present inventors have shown using bispecific molecules comprising binding sites for both MSLN and TNFRSF receptor, specifically mAb² molecules comprising two constant domain binding sites for a TNFRSF receptor, and two CDR-based antigen binding sites for MSLN that binding of the antibody molecule to both MSLN and the TNFRSF receptor, induces or enhances, T cell activation.

An antibody molecule comprising a second antigen-binding site for a TNFRSF receptor that activates immune cells, such as T cells, only on binding to the MSLN and the TNFRSF receptor, or whose immune cell activation activity is enhanced on binding to MSLN and the TNFRSF receptor, is also referred to as a conditional agonist. This immune cell activation activity is independent of binding of the antibody molecule to Fcγ receptors and/or external crosslinking agents, such as protein A or G or secondary antibodies, and therefore allows the conditional agonist activity of the antibody molecule to be targeted to sites where both MSLN and the TNFRSF are present. As MSLN is a tumour antigen, the antibody molecule may activate immune cells, such as T cells, selectively at the site of the tumour and not elsewhere in an individual.

An antibody molecule which activates immune cells, such as T cells, only on binding to a MSLN and the TNFRSF receptor, may have increased immune cell activation activity compared with antibody molecules that rely on crosslinking by other mechanisms, such as external crosslinking agents, or crosslinking via Fcγ receptor interaction. Because the activation of the TNFRSF receptor is more efficient, immune cell activation may be achieved at lower concentrations of antibody molecules described herein relative to other anti-TNFRSF antibody molecules.

Where the antibody molecule of the invention comprises a second antigen binding site for a TNFRSF receptor present on a T cell, the antibody molecule preferably induces increased activation of immune cells, such as T cells, when the antibody molecule is crosslinked, e.g. through binding to MSLN, than when the antibody molecule is not crosslinked.

The ability of an antibody molecule to activate T cells may be measured using a T cell activation assay. T cells release IL-2 on activation. A T cell activation assay may therefore measure IL-2 release to determine the level of T cell activation induced by the antibody molecule.

For example, the ability of the antibody molecule to activate T cells may be determined by measuring the concentration of the antibody molecule required to achieve half-maximal release of IL-2 by the T cells in a T cells activation assay when the antibody molecule is crosslinked. This is also referred to as the EC₅₀ of the antibody molecule. A lower EC₅₀ indicates that a lower concentration of the antibody molecule is needed to achieve half-maximal release of IL-2 by the T cells in the T cells activation assay, and thus that the antibody molecule has a higher T cell activation activity. The antibody molecule may be crosslinked using and anti-CH2 antibody, for example.

In addition, or alternatively, the ability of an antibody molecule to activate T cells may be determined by measuring the maximum concentration of IL-2 released by the T cells in a T cell activation assay in the presence of the antibody molecule, wherein the antibody molecule is crosslinked.

In a preferred embodiment, the antibody molecule (e.g. in mAb² format comprising Fcab FS22-172-003) when crosslinked e.g. via binding to NCI-H226 cells has an EC₅₀ in a T cell activation assay which is within 50-fold, 40-fold, 30-fold, 20-fold, 10-fold, or 5-fold of the EC₅₀ of FS22-172-003-AA/FS28-256-271 in the same assay, wherein FS22-172-003-AA/FS28-256-271 consists of the heavy chain of SEQ ID NO: 187 and the light chain of SEQ ID NO: 188.

For example, the antibody molecule when crosslinked may have an EC₅₀ in a primary T cell activation assay of 30 nM or less, 25 nM or less, 20 nM or less, 14 nM or less, 10 nM or less, 5 nM or less, 4 nM or less, 3 nM or less, 2 nM or less, 1.5 nM, 1 nM or 0.5 nM or less, preferably 1.5 nM or less, more preferably 1 nM, most preferably 0.5 nM or less when crosslinked.

In addition, or alternatively, the ability of an antibody molecule to activate T cells may be determined by measuring the maximum concentration of IL-2 released by the T cells in a T cell activation assay in the presence of the crosslinked antibody molecule.

In a preferred embodiment, the maximum concentration of IL-2 released by the T cells in a T cell activation assay in the presence of the antibody molecule (e.g. in mAb² format comprising Fcab FS22-172-003) when crosslinked e.g. via binding to NCI-H226 cells is within 20%, or 10% of the maximum concentration of IL-2 released by the T cells in the presence of FS22-172-003-AA/FS28-256-271 in the same assay, FS22-172-003-AA/FS28-256-271 consists of the heavy chain of SEQ ID NO: 187 and the light chain of SEQ ID NO: 188.

For example, the T cell assay may be a pan-T cell activation assay or a CD8+ T activation cell assay, depending on the TNFRSF receptor bound by the second antigen binding site. For example, a pan-T cell assay is suitable where the TNFRSF receptor is OX40, while a CD8+ T cell assay is suitable where the TNFRSF receptor is CD137.

For example, a pan-T cell activation assay may comprise isolating human PBMCs from leucocyte depletion cones. Methods for isolating PBMCs are known in the art. The T cells may then be isolated from the PBMCs. Methods for isolating T cells from PBMCs are also known in the art.

The T cell activation assay may comprise preparing the required number of T cells for example in a suitable medium, such as a T cell medium. The required number of T cells may be prepared at a concentration of 1.0×10⁶ cells/ml. T cells may then be stimulated using a suitable T cell activation reagent that provides the signals required for T cell activation. For example, the T cell activation reagent may be a reagent comprising CD3 and CD28, such as beads comprising CD3 and CD28. Isolated T cells may be incubated overnight with the T cell activation reagent to activate the T cells. Following this, the activated T cells may be washed to separate the T cells from the T cell activation reagent and resuspended in T cell medium at a suitable concentration, such as 2.0×10⁶ cells/ml. Activated T cells may then be added to plates coated with an anti-human CD3 antibody.

A suitable dilution of each test antibody molecule may be prepared and added to the wells. The T cells may then be incubated at 37° C., 5% CO₂ for 24 hours with the test antibody. Supernatants may be collected and assayed to determine the concentration of IL-2 in the supernatant. Methods for determining the concentration of IL-2 in a solution are known in the art and are described in the present examples. The concentration of human IL-2 may be plotted versus the log concentration of the antibody molecule. The resulting curves may be fitted using the log (agonist) versus response equation.

For example, a CD8+ T cell activation assay may comprise isolating human PBMCs from leucocyte depletion cones. Methods for isolating PBMCs are known in the art. The CD8+ T cells may then be isolated from the PBMCs. Methods for isolating CD8+ T cells from PBMCs are also known in the art.

The CD8+ T cells may then be added to multiwall plates coated with an anti-human CD3 antibody. A suitable dilution of each test antibody molecule may be prepared and added to the wells. The T cells may then be incubated at 37° C., 5% CO₂ for 24 hours with the test antibody. Supernatants may be collected and assayed to determine the concentration of IL-2 in the supernatant. Methods for determining the concentration of IL-2 in a solution are known in the art and are described in the present examples. The concentration of human IL-2 may be plotted versus the log concentration of the antibody molecule. The resulting curves may be fitted using the log (agonist) versus response equation.

TNFRSF receptors include CD27, CD40, EDA2R, EDAR, FAS, LTBR, RELT, TNFRSF1A, TNFRSF1B, TNFRSF4 (OX40), TNFRSF6B, TNFRSF8, TNFRSF9 (CD137), TNFRSF10A-10D, TNFRSF11A, TNFRSF11B, TNFRSF12A, TNFRSF13B, TNFRSF13C, TNFRSF14, TNFRSF17, TNFRSF18, TNFRSF19, TNFRSF21 and TNFRSF25.

In a preferred embodiment, the TNFRSF receptor is TNFRSF4 (OX40).

In an alternative preferred embodiment, the TNFRSF receptor is TNFRSF9 (CD137).

CD27 (TNFRSF7: Gene ID 939) has the reference amino acid sequence of NP_001233.1 and may be encoded by the reference nucleotide sequence of NM_001242.4. CD40 (TNFRSF5: Gene ID 958) has the reference amino acid sequence of NP_001241.1 and may be encoded by the reference nucleotide sequence of NM_001250.5. EDA2R (TNFRSF27: Gene ID 60401) has the reference amino acid sequence of NP_001186616.1 and may be encoded by the reference nucleotide sequence of NM_001199687.2. EDAR (Gene ID 10913) has the reference amino acid sequence of NP_071731.1 and may be encoded by the reference nucleotide sequence of NM_022336, 3. FAS (TNFRSF6: Gene ID 355) has the reference amino acid sequence of NP_000034.1 and may be encoded by the reference nucleotide sequence of NM_000043.5. LTBR (TNFRSF3: Gene ID 4055) has the reference amino acid sequence of NP_001257916.1 and may be encoded by the reference nucleotide sequence of NM_001270987.1. RELT (TNFRSF19L: Gene ID 84957) has the reference amino acid sequence of NP_116260.2 and may be encoded by the reference nucleotide sequence of NM_032871.3. TNFRSF1A (Gene ID 7132) has the reference amino acid sequence of NP_001056.1 and may be encoded by the reference nucleotide sequence of NM_001065.3. TNFRSF1B (Gene ID 7133) has the reference amino acid sequence of NP_001057.1 and may be encoded by the reference nucleotide sequence of NM_001066.2. TNFRSF4 (Gene ID 7293) has the reference amino acid sequence of NP_003318 and may be encoded by the reference nucleotide sequence of NM_003327). TNFRSF6B (Gene ID 8771) has the reference amino acid sequence of NP_003814.1 and may be encoded by the reference nucleotide sequence of NM_003823.3. TNFRSF8 (Gene ID 943) has the reference amino acid sequence of NP_001234.3 and may be encoded by the reference nucleotide sequence of NM_001243.4. TNFRSF9 (Gene ID 3604) has the reference amino acid sequence of NP_001552 and may be encoded by the reference nucleotide sequence of NM001561). TNFRSF10A (Gene ID 8797) has the reference amino acid sequence of NP_003835.3 and may be encoded by the reference nucleotide sequence of NM_003844.3. TNFRSF10B (Gene ID 8795) has the reference amino acid sequence of NP_003833.4 and may be encoded by the reference nucleotide sequence of NM_003842.4. TNFRSF10C (Gene ID 8794) has the reference amino acid sequence of NP_003832.2 and may be encoded by the reference nucleotide sequence of NM_003841.4. TNFRSF10D (Gene ID 8793) has the reference amino acid sequence of NP_003831.2 and may be encoded by the reference nucleotide sequence of NM_003840.4. TNFRSF11A (Gene ID 8792) has the reference amino acid sequence of XP_011524547.1 and may be encoded by the reference nucleotide sequence of XM_11526245.2. TNFRSF11B (Gene ID 4982) has the reference amino acid sequence of NP_002537.3 and may be encoded by the reference nucleotide sequence of NM_002546.3. TNFRSF12A (Gene ID 51330) has the reference amino acid sequence of NP_057723.1 and may be encoded by the reference nucleotide sequence of NM_016639.2. TNFRSF13B (Gene ID 23495) has the reference amino acid sequence of NP_0036584.1 and may be encoded by the reference nucleotide sequence of NM_012452.2. TNFRSF13C (Gene ID 115650) has the reference amino acid sequence of NP_443177.1 and may be encoded by the reference nucleotide sequence of NM_052945.3. TNFRSF14 (Gene ID 8764) has the reference amino acid sequence of NP_001284534.1 and may be encoded by the reference nucleotide sequence of NM_001297605.1. TNFRSF17 (Gene ID 608) has the reference amino acid sequence of NP_001183.2 and may be encoded by the reference nucleotide sequence of NM_001192.2. TNFRSF18 (Gene ID 8784) has the reference amino acid sequence of NP_004195.2 and may be encoded by the reference nucleotide sequence of NM_004186.1. TNFRSF19 (Gene ID 55504) has the reference amino acid sequence of NP_001191387.1 and may be encoded by the reference nucleotide sequence of NM_001204458.1. NFRSF21 (Gene ID 27242) has the reference amino acid sequence of NP_055267.1 and may be encoded by the reference nucleotide sequence of NM_014452.4. TNFRSF25 (DR3: Gene ID 8718) binds to ligand TNFSF15 (TL1A) has the reference amino acid sequence of NP_001034753.1 and may be encoded by the reference nucleotide sequence of NM_001039664.1.

In some embodiments, the antibody molecule may not comprise an antigen-binding site in a constant domain, e.g. a CH3 domain of the antibody molecule. For example, the antibody molecule may not comprise an antigen-binding site that binds to CD137 in a constant domain, such as a CH3 domain, of the antibody molecule. In particular, the antibody molecule may not comprise a CD137 antigen-binding site in a constant domain of the antibody molecule, wherein the antigen-binding site comprises modifications in one or more structural loops of the constant domain, such as one or more modifications in the AB, CD and/or EF structural loops of the constant domain. In a particular embodiment, the antibody molecule may not comprise a CD137 antigen-binding site located in a CH3 domain of the antibody molecule comprising a first sequence and a second sequence located in the AB and EF structural loops of the CH3 domain, respectively, wherein the first and second sequence have the sequence set forth in SEQ ID NO: 198 and 199, respectively [FS22-172-003]. For example the antibody molecule may not comprise the light and heavy chain sequences set forth in SEQ ID NO: 200 and 201 [FS22-172-003-AA/FS28-256-271].

The antibody molecule may be conjugated to a bioactive molecule or a detectable label. In this case, the antibody molecule may be referred to as a conjugate. Such conjugates find application in the treatment and/or diagnosis of diseases as described herein.

For example, the bioactive molecule may be an immune system modulator, such as a cytokine, preferably a human cytokine. For example, the cytokine may be a cytokine which stimulates T cell activation and/or proliferation. Examples of cytokines for conjugation to the antibody molecule include IL-2, IL-10, IL-12, IL-15, IL-21, GM-CSF and IFN-gamma.

Alternatively, the bioactive molecule may be a ligand trap, such as a ligand trap of a cytokine, e.g. of TGF-beta or IL-6.

As a further alternative, the bioactive molecule may be a ligand such as CD137L, OX40L, TRAIL, CD40L, CD27L, or GITRL.

As a further alternative, the bioactive molecule may be a drug such as an inhibitor of tubulin polymerisation (e.g. an auristatin), a tubulin depolymerisation agent (e.g. a maytansine), a DNA strand scission inducing agent (e.g. calicheamicin), a DNA alkylating agent (e.g. duocarmycin), or an RNA polymerase inhibitor (such as alpha-amanitin).

As a yet further alternatively, the bioactive molecule may be a therapeutic radioisotope.

Radioimmunotherapy is used in cancer treatment, for example. Therapeutic radioisotopes suitable for radioimmunotherapy are known in the art and include yttrium-90, iodine-131, bismuth-213, astatine-211, lutetium 177, rhenium-188, copper-67, actinium-225, iodine-125.

Suitable detectable labels which may be conjugated to antibody molecules are known in the art and include radioisotopes such as iodine-125, iodine-131, yttrium-90, indium-111 and technetium-99; fluorochromes, such as fluorescein, rhodamine, phycoerythrin, Texas Red and cyanine dye derivatives for example, Cy7 and Alexa750; chromogenic dyes, such as diaminobenzidine; latex beads; enzyme labels such as horseradish peroxidase; phosphor or laser dyes with spectrally isolated absorption or emission characteristics; and chemical moieties, such as biotin, which may be detected via binding to a specific cognate detectable moiety, e.g. labelled avidin.

The antibody molecule may be conjugated to the bioactive molecule or detectable label by means of any suitable covalent or non-covalent linkage, such as a disulphide or peptide bond. Where the bioactive molecule is a cytokine, the cytokine may be joined to the antibody molecule by means of a peptide linker. Suitable peptide linkers are known in the art and may be 5 to 25, 5 to 20, 5 to 15, 10 to 25, 10 to 20, or 10 to 15 amino acids in length.

In some embodiments, the bioactive molecule may be conjugated to the antibody molecule by a cleavable linker. The linker may allow release of the bioactive molecule from the antibody molecule at a site of therapy. Linkers may include amide bonds (e.g. peptidic linkers), disulphide bonds or hydrazones. Peptide linkers for example may be cleaved by site specific proteases, disulphide bonds may be cleaved by the reducing environment of the cytosol and hydrazones may be cleaved by acid-mediated hydrolysis.

The conjugate may be a fusion protein comprising the antibody molecule and the bioactive molecule. In this case the bioactive molecule may be conjugated to the antibody molecule by means of a peptide linker or peptide bond. Where the antibody molecule is a multichain molecule, such as where the antibody molecule is or comprises an Fcab or is a mAb², the bioactive molecule may be conjugated to one or more chains of the antibody molecule. For example, the bioactive molecule may be conjugated to one or both of the heavy chains of the mAb² molecule. Fusion proteins have the advantage of being easier to produce and purify, facilitating the production of clinical-grade material.

The invention also provides an isolated nucleic acid molecule or molecules encoding an antibody molecule of the invention. The skilled person would have no difficulty in preparing such nucleic acid molecules using methods well-known in the art.

The nucleic acid molecule or molecules may encode the VH domain and/or VL domain, preferably the VH domain and VL domain of: antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, or FS28-024, preferably antibody FS28-256-271, or FS28-024-052, most preferably antibody FS28-256-271.

For example, a nucleic acid molecule which encodes the VH domain of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024 is set forth in SEQ ID NO: 181, 40, 110, 110, 122, 110, 122, 110, 64, 64, 116, 122, 70, 31, 50, and 9, respectively.

A nucleic acid molecule which encodes the VL domain of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024 is set forth in SEQ ID NO: 57, 19, 94, 80, 94, 54, 54, 57, 94, 54, 80, 80, 80, 19, 19, and 19 respectively.

In a preferred embodiment, the nucleic acid molecule(s) encode the heavy chain and/or light chain, preferably the heavy chain and light chain of: antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, or FS28-024, preferably antibody FS28-256-271, or FS28-024-052, most preferably antibody FS28-256-271.

For example, a nucleic acid molecule which encodes the heavy chain (with LALA mutation) of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024 is set forth in SEQ ID NO: 179, 38, 108, 108, 126, 108, 126, 108, 84, 84, 120, 126, 68, 29, 48, and 7, respectively.

For example, a nucleic acid molecule which encodes the heavy chain (without LALA mutation) of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024 is set forth in SEQ ID NO: 177, 36, 106, 106, 124, 106, 124, 106, 82, 88, 118, 124, 66, 27, 46, and 6 respectively.

A nucleic acid molecule which encodes the light chain of antibody FS28-256-271, FS28-024-052, FS28-256-021, FS28-256-012, FS28-256-023, FS28-256-024, FS28-256-026, FS28-256-027, FS28-256-001, FS28-256-005, FS28-256-014, FS28-256-018, FS28-256, FS28-024-051, FS28-024-053, and FS28-024 is set forth in SEQ ID NO: 96, 17, 92, 78, 92, 91, 91, 96, 92, 91, 78, 78, 78, 17, 17, and 17, respectively.

Where the nucleic acid encodes the VH and VL domain, or heavy and light chain, of an antibody molecule of the invention, the two domains or chains may be encoded on two separate nucleic acid molecules.

An isolated nucleic acid molecule may be used to express an antibody molecule of the invention. The nucleic acid will generally be provided in the form of a recombinant vector for expression. Another aspect of the invention thus provides a vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g. phage, or phagemid, as appropriate.

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. A preferred host cell is a mammalian cell, such as a CHO, NS0, or HEK cell, for example a HEK293 cell.

Another aspect of the invention provides a method of producing an antibody molecule of the invention comprising expressing a nucleic acid encoding the antibody molecule in a host cell and optionally isolating and/or purifying the antibody molecule thus produced. Methods for culturing host cells are well-known in the art. The method may further comprise isolating and/or purifying the antibody molecule. Techniques for the purification of recombinant antibody molecules are well-known in the art and include, for example HPLC, FPLC or affinity chromatography, e.g. using Protein A or Protein L. In some embodiments, purification may be performed using an affinity tag on antibody molecule. The method may also comprise formulating the antibody molecule into a pharmaceutical composition, optionally with a pharmaceutically acceptable excipient or other substance as described below.

As explained above, MSLN is expressed on the surface of tumour cells and high expression levels of soluble MSLN have been correlated with poor prognosis in several cancers. Anti-MSLN antibodies have been investigated as anti-cancer therapeutics. These anti-MSLN antibodies either induce direct cell killing through their ADCC activity or are used in the form of ADCs.

The antibody molecules described herein are therefore expected to find application in the treatment of cancer. Related aspects of the invention thus provide:
(i) an antibody molecule described herein for use in a method of treating cancer in an individual,
(ii) the use of an antibody molecule described herein in the manufacture of a medicament for use in the treatment of cancer in an individual; and,
(iv) a method of treating cancer in an individual, wherein the method comprises administering to the individual a therapeutically effective amount of an antibody molecule as described herein.

The individual may be a patient, preferably a human patient.

The antibody molecules of the invention have been shown to preferentially bind to MSLN present on the surface of a cancer cell as compared to soluble MSLN. The cancer to be treated using an antibody molecule of the invention therefore preferably expresses, or has been determined to express, MSLN. More preferably, cells of the cancer to be treated comprise, or have been determined to comprise, MSLN at their cell surface, i.e. to comprise cell-surface bound MSLN.

Where the antibody molecule comprises a second antigen-binding site for an immune cell antigen, such as a TNFRSF member, e.g. in a constant domain of the antibody molecule, the cancer preferably comprises, or has been determined to comprise, tumour infiltrating lymphocytes (TILs) that express the TNFRSF member. Specifically, the TILs preferably comprise, or have been determined to comprise, the TNFRSF member on their cell surface.

Methods for determining the presence of an antigen on a cell surface are known in the art and include, for example, flow cytometry.

The cancer may be a primary or a secondary cancer. Thus, an antibody molecule as described herein may be for use in a method of treating cancer in an individual, wherein the cancer is a primary tumour and/or a tumour metastasis.

The cancer to be treated using an antibody molecule of the invention may be a solid cancer.

The cancer may be selected from the group consisting of: mesothelioma, pancreatic cancer, ovarian cancer, lung cancer (such as small-cell lung cancer and non-small cell lung cancer), oesophageal cancer, breast cancer, gastric cancer, cholangiocarcinoma, colon cancer, thymic carcinoma, endometrial cancer, head and neck cancer, sarcoma (such as biphasic synovial sarcoma, Kaposi's sarcoma, osteogenic sarcoma, rhabdomyosarcoma, or soft-tissue sarcoma), desmoplastic small round cell tumours, leukaemia (such as acute lymphocytic leukaemia, chronic lymphocytic leukaemia, acute granulocytic leukaemia, chronic granulocytic leukaemia, hairy cell leukaemia, or myeloid leukaemia), adrenal cortex cancer, bladder cancer, brain cancer, cervical cancer, cervical hyperplasia, testicular choriocarcinoma, essential thrombocytosis, genitourinary carcinoma, glioma, glioblastoma, lymphoma (such as Hodgkin's disease or non-Hodgkin's lymphoma), malignant carcinoid carcinoma, malignant hypercalcemia, melanoma (also referred to as malignant melanoma), malignant pancreatic insulinoma, medullary thyroid carcinoma, multiple myeloma, mycosis fungoides, neuroblastoma, polycythemia vera, primary brain carcinoma, primary macroglobulinemia, prostate cancer, renal cell cancer, skin cancer, squamous cell cancer, stomach cancer, testicular cancer, thyroid cancer, and Wilms' tumor.

Preferably, the cancer is selected from the group consisting of: mesothelioma, pancreatic cancer, ovarian cancer, lung cancer, oesophageal cancer, breast cancer, gastric cancer, cholangiocarcinoma, colon cancer, thymic carcinoma, endometrial cancer, head and neck cancer, biphasic synovial sarcomas, and desmoplastic small round cell tumours.

More preferably, the cancer is selected from the group consisting of: mesothelioma, pancreatic cancer, ovarian cancer, and lung cancer.

Cancer is characterised by the abnormal proliferation of malignant cancer cells. Where a particular type of cancer, such as breast cancer, is referred to, this refers to an abnormal proliferation of malignant cells of the relevant tissue, such as breast tissue. A secondary cancer which is located in the breast but is the result of abnormal proliferation of malignant cells of another tissue, such as ovarian tissue, is not a breast cancer as referred to herein but an ovarian cancer.

In the context of cancer, treatment may include inhibiting cancer growth, including complete cancer remission, and/or inhibiting cancer metastasis, as well as inhibiting cancer recurrence. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of anti-cancer immune cells or other anti-cancer immune responses, and a decrease in levels of tumour-specific antigens. Activating or enhancing immune responses to cancerous tumours in an individual may improve the capacity of the individual to resist cancer growth, in particular growth of a cancer already present in the subject and/or decrease the propensity for cancer growth in the individual.

Whilst an antibody molecule may be administered alone, antibody molecules will usually be administered in the form of a pharmaceutical composition, which may comprise at least one component in addition to the antibody molecule. Another aspect of the invention therefore provides a pharmaceutical composition comprising an antibody molecule as described herein. A method comprising formulating an antibody molecule into a pharmaceutical composition is also provided.

Pharmaceutical compositions may comprise, in addition to the antibody molecule, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

For parenteral, for example subcutaneous or intravenous administration, e.g. by injection, the pharmaceutical composition comprising the antibody molecule may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

In some embodiments, antibody molecules may be provided in a lyophilised form for reconstitution prior to administration. For example, lyophilised antibody molecules may be re-constituted in sterile water and mixed with saline prior to administration to an individual.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. Appropriate doses of antibody molecules are well known in the art (Ledermann et al., 1991; Bagshawe et al., 1991). Specific dosages indicated herein, or in the Physician's Desk Reference (2003) as appropriate for an antibody molecule being administered, may be used. A therapeutically effective amount or suitable dose of an antibody molecule can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the antibody molecule.

A typical antibody dose is in the range 100 μg to 1 g for systemic applications, and 1 μg to 1 mg for topical applications. An initial higher loading dose, followed by one or more lower doses, may be administered. This is a dose for a single treatment of an adult individual, which may be proportionally adjusted for children and infants, and also adjusted for other antibody formats in proportion to molecular weight.

Treatments may be repeated at daily, twice-weekly, weekly or monthly intervals, at the discretion of the physician. The treatment schedule for an individual may be dependent on the pharmacokinetic and pharmacodynamic properties of the antibody composition, the route of administration and the nature of the condition being treated.

Treatment may be periodic, and the period between administrations may be about two weeks or more, e.g. about three weeks or more, about four weeks or more, about once a month or more, about five weeks or more, or about six weeks or more. For example, treatment may be every two to four weeks or every four to eight weeks. Suitable formulations and routes of administration are described above.

In the context of cancer treatment, an antibody molecule as described herein may be administered to an individual in combination with another anti-cancer therapy or therapeutic agent, such as an anti-cancer therapy or therapeutic agent which has been shown to be suitable, or is expected to be suitable, for the treatment of the cancer in question. For example, the antibody molecule may be administered to the individual in combination with a chemotherapeutic agent, radiotherapy, an immunotherapeutic agent, an anti-tumour vaccine, an oncolytic virus, an adoptive cell transfer (ACT) therapy (such as adoptive NK cell therapy or therapy with chimeric antigen receptor (CAR) T-cells, autologous tumour infiltrating lymphocytes (TILs), or gamma/delta T cells, or an agent for hormone therapy.

Without wishing to be bound by theory, it is thought that the antibody molecule as described herein, wherein the antibody molecules comprises a second antigen-binding site for an immune cell antigen, such as a TNFRSF receptor, may act as an adjuvant in anti-cancer therapy. Specifically, it is thought that administration of the antibody molecule to an in individual in combination with chemotherapy and/or radiotherapy, or in combination with an anti-tumour vaccine, for example, will trigger a greater immune response against the cancer than is achieved with chemotherapy and/or radiotherapy, or with an anti-tumour vaccine, alone.

One or more chemotherapeutic agents for administration in combination with an antibody molecule as described herein may be selected from the group consisting of: taxanes, cytotoxic antibiotics, tyrosine kinase inhibitors, PARP inhibitors, B-Raf enzyme inhibitors, MEK inhibitors, c-MET inhibitors, VEGFR inhibitors, PDGFR inhibitors, alkylating agents, platinum analogues, nucleoside analogues, antifolates, thalidomide derivatives, antineoplastic chemotherapeutic agents and others. Taxanes include docetaxel, paclitaxel and nab-paclitaxel; cytotoxic antibiotics include actinomycin, bleomycin, and anthracyclines such as doxorubicin, mitoxantrone and valrubicin; tyrosine kinase inhibitors include erlotinib, gefitinib, axitinib, PLX3397, imatinib, cobemitinib and trametinib; PARP inhibitors include piraparib; B-Raf enzyme inhibitors include vemurafenib and dabrafenib; alkylating agents include dacarbazine, cyclophosphamide and temozolomide; platinum analogues include carboplatin, cisplatin and oxaliplatin; nucleoside analogues include azacitidine, capecitabine, fludarabine, fluorouracil and gemcitabine; antifolates include methotrexate and pemetrexed. Other chemotherapeutic agents suitable for use in the present invention include defactinib, entinostat, eribulin, irinotecan and vinblastine.

Preferred therapeutic agents for administration with an antibody molecule as described herein are pentostatin, cyclophosphamide, cis-platin, pemetrexed, paclitaxel, carboplatin, gemcitabine, doxorubicin, vinorelbine, docetaxel, or etoposide

A radiotherapy for administration in combination with an antibody molecule as described herein may be external beam radiotherapy (such as intensity-modulated radiotherapy (IMRT), stereotactic body radiotherapy (SBRT), image-guided radiotherapy (IGRT), intra-operative radiotherapy (IORT), electron therapy or electron beam therapy (EBT), superficial radiotherapy (SRT)), or internal radiotherapy (such as brachytherapy, radioisotope or radionuclide therapy, SIRT. Preferably, the radiotherapy is conventional external beam radiotherapy, external beam radiation therapy (EBRT), stereotactic radiotherapy, or brachytherapy

An immunotherapeutic agent for administration in combination with an antibody molecule as described herein may be a therapeutic antibody molecule, nucleic acid, cytokine, or cytokine-based therapy. For example, the therapeutic antibody molecule may bind to an immune regulatory molecule, e.g. an inhibitory checkpoint molecule or an immune costimulatory molecule, a receptor of the innate immune system, or a tumour antigen, e.g. a cell surface tumour antigen or a soluble tumour antigen. Examples of immune regulatory molecules to which the therapeutic antibody molecule may bind include inhibitory checkpoint molecules, such as CTLA-4, LAG-3, TIGIT, TIM-3, VISTA, PD-L1, PD-1, or KIR, immune costimulatory molecules, such as OX40, CD40, CD137, GITR, CD27, or ICOS, other immune regulatory molecules such as CD47, CD73, CSF-1R, HVEM, TGFB, or CSF-1. Examples of receptors of the innate immune system to which the therapeutic antibody molecule may bind include TLR1, TLR2, TLR4, TLR5, TLR7, TLR9, RIG-1-like receptors (e.g. RIG-1 and MDA-5), and STING.

The nucleic acid for administration in combination with an antibody molecule as described herein may be an siRNA.

The cytokines or cytokine-based therapy may be selected from the group consisting of: IL-2, prodrug of conjugated IL-2, GM-CSF, IL-7, IL-12, IL-9, IL-15, IL-18, IL-21, and type I interferon.

Anti-tumour vaccines for the treatment of cancer have both been implemented in the clinic and discussed in detail within scientific literature (such as Rosenberg, S. 2000). This mainly involves strategies to prompt the immune system to respond to various cellular markers expressed by autologous or allogenic cancer cells by using those cells as a vaccination method, both with or without granulocyte-macrophage colony-stimulating factor (GM-CSF). GM-CSF provokes a strong response in antigen presentation and works particularly well when employed with said strategies.

The chemotherapeutic agent, radiotherapy, immunotherapeutic agent, anti-tumour vaccine, oncolytic virus, ACT therapy, or agent for hormone therapy is preferably a chemotherapeutic agent, radiotherapy, immunotherapeutic agent, anti-tumour vaccine, oncolytic virus, ACT therapy, or agent for hormone therapy for the cancer in question, i.e. a chemotherapeutic agent, radiotherapy, immunotherapeutic agent, anti-tumour vaccine, oncolytic virus, ACT therapy, or agent for hormone therapy which has been shown to be effective in the treatment of the cancer in question. The selection of a suitable chemotherapeutic agent, radiotherapy, immunotherapeutic agent, anti-tumour vaccine, oncolytic virus, ACT therapy, or agent for hormone therapy which has been shown to be effective for the cancer in question is well within the capabilities of the skilled practitioner.

The antibody molecules of the invention may be useful in the detection MSLN, in particular in the detection of immobilised MSLN, such as cell-surface bound MSLN. The antibody molecule may be conjugated to a detectable label as described elsewhere herein.

Thus, the present invention relates to the use of an antibody molecule for detecting the presence of immobilised MSLN, preferably the presence of cells comprising MSLN at their cell surface, in a sample.

Also provided is an in vitro method of detecting MSLN, wherein the method comprises incubating the antibody molecule with a sample of interest, and detecting binding of the antibody molecule to the sample, wherein binding of the antibody to the sample indicates the presence of immobilised MSLN. Binding of the antibody molecule to a sample may be detected using an ELISA, for example.

In a preferred embodiment, the present invention relates to an in vitro method of detecting cells comprising MSLN at their cell surface, wherein the method comprises incubating the antibody molecule with a cell sample of interest, and determining binding of the antibody molecule to cells present in the sample, wherein binding of the antibody to cells present in sample indicates the presence of cells comprising MSLN at their cell surface. Methods for detecting binding of an antibody molecule to cells are known in the art and include ELISAs, and flow-cytometry.

The antibody molecules of the invention may find application in the detection, diagnosis, and/or prognosis of cancer. The cancer may be a cancer which can be treated with an antibody molecule of the invention as described herein.

Related aspects of the invention thus provide;
(i) an antibody molecule described herein for use in a method of detecting cancer, diagnosing cancer, determining cancer prognosis, or monitoring cancer prognosis in an individual;
(ii) the use of an antibody molecule described herein in the manufacture of a diagnostic product for use in the detecting cancer, diagnosing cancer, determining cancer prognosis, or monitoring cancer prognosis;
(iii) a method of detecting cancer, diagnosing cancer, determining cancer prognosis, or monitoring cancer prognosis in an individual using an antibody molecule as described herein; and
(iv) a kit for use in a method of detecting, diagnosing, prognosis, or monitoring the prognosis of cancer in an individual, the kit comprising an antibody molecule as described herein.

The method may comprise administering an antibody molecule of the invention to an individual and determining the presence of the antibody molecule at a site in the body of the individual, wherein the presence of the antibody molecule at a site in the body indicates the presence of a tumour, in particular the presence of a tumour comprising cells expressing MSLN at their cell surface.

In a preferred embodiment, the method comprises determining the presence of cells expressing MSLN at their cell surface in a sample obtained from an individual, wherein the presence of cells expressing MSLN at their cell surface indicates that the individual has cancer.

In an alternative preferred embodiment, the method comprises determining the presence of tumour cells expressing MSLN at their cell surface in a tumour sample obtained from an individual, wherein the presence of tumour cells expressing MSLN at their cell surface indicates that the individual has a worse prognosis, such as a higher risk of cancer metastasis, than an individual with the same cancer which does not comprise cells expressing MSLN at their cell surface.

The cancer may be a cancer as referred to herein. Preferably, the cancer is selected from the group consisting of: mesothelioma, pancreatic cancer, ovarian cancer, lung cancer, oesophageal cancer, breast cancer, gastric cancer, cholangiocarcinoma, colon cancer, thymic carcinoma, endometrial cancer, head and neck cancer, biphasic synovial sarcomas, and desmoplastic small round cell tumours. More preferably, the cancer is selected from the group consisting of: mesothelioma, pancreatic cancer, ovarian cancer, and lung cancer.

Further aspects and embodiments of the invention will be apparent to those skilled in the art given the present disclosure including the following experimental exemplification.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Other aspects and embodiments of the invention provide the aspects and embodiments described above with the term “comprising” replaced by the term “consisting of” or “consisting essentially of”, unless the context dictates otherwise.

Certain aspects and embodiments of the invention will now be illustrated by way of example and with reference to the figures described above.

### EXAMPLES

### Example 1: Isolation of Anti-Human MSLN Antibodies: Antigens, Selections and Screening

Mesothelin is a glycosylphosphatidylinositol (GPI)-linked glycoprotein synthesized as a 69 kDa precursor and proteolytically processed into a 30 kDa NH2-terminal secreted form (referred to as megakaryocyte potentiating factor or MPF) and a 40 kDa membrane-bound mesothelin (MSLN). Soluble forms of MSLN, shed from the tumour cell surface and generated by alternative splicing or tumour necrosis factor-α-converting enzymes (TACE) of the membrane-bound MSLN, are found in patient serum. This tumour-shed antigen is known to create a ‘sink’ which can act as a decoy for therapeutic antibodies (Lee et al, 2018) such that this must be overcome to allow the antibodies to bind to MSLN on the tumour. To avoid this sink effect, the inventors set out to isolate novel anti-mesothelin antibodies that preferentially bound to immobilised MSLN compared to soluble MSLN, with the intention that this would translate to preferential binding to membrane-bound MSLN over soluble MSLN. To this end, different forms of MSLN antigens were employed in phage selections and subsequent screening campaigns.

### 1.1 Production of Human, Cynomolgus and Mouse Mesothelin Antigens

Recombinant biotinylated human MSLN-His antigen, designated ‘hMSLN-His Acro’, was obtained from Acrobiosystems (cat no MSN-H8223) which lacks the C-terminal 18 amino acids. To maximise the diversity of binders selected across the whole of the antigen, full-length monomeric human MSLN antigen was generated and biotinylated in house for phage selections. Cynomolgus and mouse MSLN were produced to allow the isolation of binders that were capable of binding to human as well as cyno MSLN, and also for the isolation of murine MSLN binders respectively.

Briefly, MSLN antigens were produced by cloning DNA encoding human (SEQ ID NO: 169) (hMSLN-His-Avi), cynomolgus (SEQ ID NO: 170) (cMSLN-His-Avi) or mouse (SED ID NO: 171) (mMSLN-His-Avi) MSLN along with six C-terminal histidine residues and an Avi sequence into modified pFUSE vectors (Invivogen cat no pfuse-mg2afc2) using EcoRI-HF and BamHI-HF restriction enzymes. The vectors were transfected into HEK293-6E cells (National Research Council of Canada), and expressed MSLN was purified using a HisTrap™ excel nickel column (GE LifeSciences, 29048586). Each of the antigens was biotinylated using a BirA biotin-biotin protein ligase reaction kit (Avidity LLC, BirA500) to produce monomeric MSLN antigens labelled with a single biotin molecule. Specifically, five to ten mg of antigen was mixed with BirA enzyme mix to a molar ratio of enzyme to substrate of 1:50. Additives were then added in accordance to the manufacturer's recommendations, incubated overnight at room temperature and recombinant human, cyno or mouse MSLN was subsequently purified using a HisTrap™ excel nickel column (GE LifeSciences, 29048586) to remove excess free biotin.

The biophysical properties of each antigen were characterised by SEC-HPLC analysis to determine whether aggregates were present and by PAGE to verify the size of the molecules. SEC-HPLC of these antigens showed less than 10% aggregation and PAGE verified that the antigens were monomeric. ELISA and surface plasmon resonance (SPR) were used to confirm that the biotinylated MSLN antigens could be bound by MSLN-specific positive control antibodies (SS1, see section 1.3; Hassan et al 2002 and MOR6626 see section 7.1, Patent Publication No. WO 2009/068204 A1). Based on this data all antigens were deemed suitable for naïve selections.

### 1.2 Phagemid Library Selections

Synthetic naïve phagemid libraries displaying the human Fab domain of germlines with randomised amino acids in the CDR1, CDR2 and CDR3 (MSM Technologies) were used for selections with the MSLN antigens described in section 1.1.

Fab libraries were initially selected in multiple campaigns each in three or four rounds using Streptavidin Dynabeads (Thermo Fisher, 11205D), Neutravidin-binding protein coupled to Dynabeads (Thermo Fisher, 31000) or anti-His Dynabeads (Thermo Fisher, 10103D) to isolate the phage bound to biotinylated human, cyno or mouse MSLN-His-Avi or hMSLN-His Acro. Selection campaigns were also performed using full-length MSLN antigens produced in-house in which human MSLN selection rounds were alternated with cyno MSLN antigen with an aim to isolate human and cyno cross-reactive clones. Selections for binders to mouse MSLN (R&D mMSLN-His 8604-MS) were also performed. Standard phage selection and phage recovery procedures were used.

In an effort to obtain clones that bind to different regions of MSLN antigen, an epitope masking strategy was adopted using anti-MSLN antibodies from the initial selection campaigns described above. Briefly, a first round of selection of the naïve Fab libraries was performed using biotinylated human MSLN-His-Avi at 500 nM. In round 2 and 3 the phage binding of biotinylated cyno MSLN-His-Avi at 500 nM (round 2) or 100 nM (round 3) was tested in the presence of a mixture of naïve anti-mesothelin mAb proteins isolated from the initial selection campaign (FS28-004, FS28-024, FS28-026, FS28-091 and FS28-97, 500 nM of each mAb). These epitope masking selections resulted in reduced output titers, indicating that the selection strategy was working as fewer binders were identified. This led to the identification of clones (FS28-243, FS28-255 and FS28-256, see section 2.1.3) that target additional areas of MSLN compared to the clones from the earlier selection campaign.

### 1.3 Screening to Identify Anti-MSLN Antibodies

About 2000 clones from round 3 and 4 outputs of all selections were screened by phage ELISA for binding to 25 nM immobilised biotinylated hMSLN-His Acro, full-length biotinylated human or cyno MSLN-His-Avi, consistent with the antigen used in the round of selections. Streptavidin plates or plates immobilised with irrelevant biotinylated His tagged antigens were included as negative controls. Clones which had a MSLN binding signal at least 4-fold higher than signal to negative controls were selected and their variable regions sequenced, leading to the identification of 156 unique VH/VL sequence combinations, which were subsequently selected for soluble expression. Clones were chosen from all selection campaigns including the epitope masking selections. For each clone, the VH and VL were individually cloned into a pTT5 expression vector (National Research Council of Canada) containing either CH1, CH2 (with a LALA mutation in the CH2 domain (Bruhns et al., 2009; Hezareh et al., 2001) and CH3 domain, or CL domains respectively. The resulting pTT5-FS28 VH with LALA mutation (AA) and pTT5-FS28 VL vectors were transiently cotransfected into HEK293-6E cells and the clones were produced as complete IgG1 molecules. The antibodies were either kept in supernatant or purified by mAb Select SuRe Protein A columns and subjected to further testing as described below. Using the same method, the VH and VL regions of SS1 and anti-hen egg white lysozyme antibody HeID1.3 were cloned and expressed in IgG1 LALA format, yielding G1-AA/SS1 (SEQ ID No 167 and 168) and G1-AA/HeID1.3 (SEQ ID No 165 and 166), to serve as positive and negative controls respectively.

### Example 2: Identification of Panel of Naïve Anti-MSLN mAbs

Anti-mesothelin mAbs have a broad range of potential applications including their use as therapeutics capable of inducing ADCC, as a tumour targeting arm for the delivery of immunotoxin, ADC and for the generation of bispecific antibodies, amongst others. The desired characteristics of the anti-mesothelin mAbs are dependent on the application and the inventors therefore set out to identify a panel of mAbs that bound to membrane-bound human and cyno MSLN, with a variety of affinities, and that were able to target different areas of MSLN. To this end, a set of screening assays including ELISA, Biacore blocking assays and cell binding were performed as well as assays in which the binding regions to MSLN were compared.

### 2.1 Screening for Binding to Recombinant MSLN

### 2.1.1 Binding ELISA

HEK293-6E supernatants containing soluble anti-MSLN binding clones or purified clones were screened for binding to human MSLN-His Acro, hMSLN-His-Avi and, for some campaigns, cMSLN-His-Avi by ELISA. Briefly, hMSLN-His Acro, hMSLN-His-Avi, cMSLN-His-Avi or an irrelevant His tagged antigen were coated on maxisorp plates at 25 nM overnight at 4° C. The next day, plates were blocked with 300 μl PBS containing 0.05% Tween20 and 2% Marvel milk (Marvel dried milk). Anti-MSLN mAb containing supernatants or purified proteins were incubated for 1 hour at room temperature and their binding was detected with a mouse anti human Fc-IgG antibody conjugated to horse radish peroxidase (HRP). Clones that showed binding to the irrelevant antigen or were not cross reactive to both the human and cynomolgus antigens were discarded. In addition, clones that bound to the truncated MSLN antigen, hMSLN-His Acro, but not to the full length MSLN antigen, MSLN-His-Avi, were also not taken forward as it was expected that the full-length antigen would be more representative of antigen conformation on the cell surface of MSLN-expressing cells.

Mesothelin is a glycoprotein and its glycosylation pattern might vary depending on species and tissue. To ensure that the antibodies had specificity for binding to MSLN, the anti-MSLN binding clones were also tested for differential binding to glycosylated and deglycosylated MSLN. Biotinylated hMSLN-His-Avi was deglycosylated using PNGase F enzyme (NEB, P0704L) for 24 hr at 37° C., purified using Amicon ultra centrifugal filters (Millipore, UFC901024) and coated on maxisorp plates at 25 nM. ELISA binding of the unique anti-mesothelin mAbs to MSLN was detected using mouse anti-human Fc-IgG-HRP (Sigma, A0170). Clones that showed a more than 2-fold reduction in binding signal to deglycosylated compared to glycosylated MSLN antigen were excluded from the panel of naïve anti-MSLN binding mAbs.

### 2.1.2 BIAcore Screening

Human MSLN-His-Avi was immobilised on flow cell 2 on a CM5 Series S BIAcore sensor chip (GE Healthcare, BR100530) using an amine coupling kit (GE Healthcare, BR10050) to approximately 200 response units (RU). Flow cell 1 was left blank for subtraction. HEK293-6E supernatants or purified proteins were adjusted with HBS-EP+ (GE Healthcare) to approximately 50 nM anti-MSLN mAb per sample. Samples were injected over flow cell 1 and 2 for 2.5 min at 30 μl/min and then allowed to dissociate in HBS-EP buffer for 2.5 min. Regeneration was achieved by injecting 10 mM glycine pH 1.5 (GE Healthcare, BR100354) for 30 seconds at a rate of 30 μl/min. Subtracted data (flow cell 2−flow cell 1) were analysed using BIAevaluation 3.2 Software (GE Healthcare). Of the 86 clones tested in this assay from the binding ELISA, 39 clones showed a binding response of greater than 10 RU at 50 nM and were therefore selected for re-expression, purification and further screening.

### 2.1.3 Binning of Antibodies Based on the Region of MSLN Bound

Based on the ELISA and Biacore screening data, clones were then tested in binning assays in which the binding of the mAbs to human MSLN was tested in the presence of another mAb by Bio-Layer Interferometry (BLI) on an Octet (ForteBio).

Briefly, biotinylated hMSLN-His-Avi (5 μg/ml) was bound to streptavidin tips (ForteBIO, 18-5020) for 5 min. G1-AA/SS1 was diluted to 200 nM in 1× kinetic buffer (FortéBIO, 18-1092) and allowed to bind to hMSLN-His-Avi for 5 minutes. Next, binding of a mixture containing 200 nM of the test mAb and 200 nM of G1-AA/SS1, was assessed for 5 minutes. This was compared with the binding of the test mAb to hMSLN-His-Avi in the absence of bound G1-AA/SS1 to determine the maximal extent of possible binding in the absence of SS1 (i.e. where there was no competition for binding). If both antibodies competed for binding to the same region of MSLN, the test antibody would not be able to bind.

These binning experiments with G1-AA/SS1 revealed that the majority (19 out of 23) of the FS28-lineage antibodies tested were unable to bind MSLN in the presence of G1-AA/SS1 and could therefore be attributed to bind a similar region as G1-AA/SS1. The MSLN binding site for the SS1 antibody in Fab format has been reported (Ma et al., 2012) and is defined as an N-terminal region comprising amino acids 7 to 64 that are also involved in MUC16 binding. The fact that most anti-MSLN binders isolated bound to this or a similar region suggests that these amino acids are well exposed in the recombinant antigens. Four other clones, FS28-185, FS28-243, FS28-255 and FS28-256, were identified that showed partial or no competition with G1-AA/SS1 for binding to MSLN Binning of these clones against each other revealed that these clones represented two additional independent bins, i.e. were capable of binding to a further two different regions of MSLN. FS28-185, FS28-243 and FS28-255 were all assigned to one bin (‘bin 2’) whereas FS28-256 was assigned to a separate bin (‘bin 3’). These results showed that the epitope masking selections in section 1.2 were successful as antibodies which bound to multiple regions of MSLN were identified.

### 2.1.4 Affinities

For each bin described in 2.1.3, the binding kinetics were determined using the same method as described in section 2.1.2, except that human or cyno MSLN-His-Avi was immobilised at 50 or 100 RU. Clones were tested at a concentration range of 81 nM to 0.33 nM in 3-fold dilutions. The binders were ranked and the best from each bin were selected: FS28-024, FS28-026 and FS28-091 all from bin 1, FS28-185 from bin 2 and FS28-256 from bin 3. All of these clones were shown to be cyno cross-reactive, but affinities were not calculated under these test conditions. The affinities of the selected antibodies are shown in Table 1, which shows that affinities obtained at 50 RU of immobilised MSLN were lower than those at 100 RU of immobilised MSLN antigen, showing increased binding at higher levels of MSLN.

TABLE 1
Affinities of naïve anti-mesothelin
mAbs to immobilised human mesothelin

| mAb (in | Affinity to immobilised human MSLN-His-Avi K_{D} (nM) | |
|---|---|---|
| G1-AA format) | 50 RU | 100 RU |
| FS28-024 | 0.77 | 0.23 |
| FS28-026 | 0.21 | 0.12 |
| FS28-091 | 1.24 | 0.76 |
| FS28-185 | 37.00 | 29.57 |
| FS28-256 | 26.20 | 23.16 |

### 2.2 MUC16-MSLN Blocking Assays

The N-terminal region of MSLN (amino acids 296-359) has been reported to interact with glycoprotein MUC16 and this interaction may play a role in cancer cell adhesion (Kaneko et al., 2009). FS28-024, FS28-026, FS28-091, FS28-185 and FS28-256 were tested for their ability to block the binding of MUC16 to mesothelin in a blocking assay. SS1 is known from the literature to block MUC16 binding to MSLN (Ma et al., 2012). As previously described, we converted this to a G1-AA/SS1 format with the expectation that this would not impact its ability to block MUC16 binding. G1-AA/SS1 and a control IgG1 antibody (G1-AA/HeID1.3) were included as positive and negative controls respectively.

Briefly, recombinant human MUC16 (R&D Systems, 5809-MU-050) was coated on maxisorp plates at 0.65 μg/ml in 1×PBS overnight at 4° C. Plates were washed 3 times with 1×PBS and blocked with 300 μl PBS containing 2% Tween20 and 2% Marvel Milk. A concentration of anti-MSLN mAbs (0.23 nM to 500 nM, 3-fold dilutions) were pre-mixed with biotinylated hMSLN-His-Avi antigen (final concentration 2 μg/ml) in a volume of 100 μl for 1 hr at room temperature. After removal of the blocking solution, the mAb/MSLN mixture was added to the plates and incubated for 1 hr at room temperature. Plates were washed 3 times with PBST (1×PBS and 0.05% Tween20) and incubated with streptavidin-HRP (Thermo Scientific, 21126, 1:1000 dilution in 1×PBS) for 1 hr at room temperature. Finally, plates were washed 3 times with PBST and 3 times with PBS. MSLN bound to MUC16 was visualised by adding 100 μl TMB for 15 min, followed by 100 μl 1M sulphuric acid solution. Absorbances were read at 450-630 nm (Gen5 software, BioTek).

TABLE 2
Activity of naïve anti-mesothelin
mAbs in MUC16-MSLN blocking assay

| mAb (in | MUC16-MSLN blocking activity |
|---|---|
| G1-AA format) | IC₅₀ (nM) |
| FS28-024 | 2.9 |
| FS28-026 | 5.2 |
| FS28-091 | 4.4 |
| FS28-185 | Enhanced binding |
| FS28-256 | No activity |
| SS1 | 4.8 |
| HelD1.3 | No activity |

Bin 1 clones FS28-024, FS28-026 and FS28-091 showed dose-dependent blocking of the MUC16-MSLN interaction with IC₅₀ of 2.9 nM, 5.2 nM and 4.4 nM respectively (Table 2). The observed blocking activity was similar to that of G1-AA/SS1. FS28-256 did not show any blocking activity similarly to the negative control G1-AA/HeID1.3. Whereas FS28-185 promoted the binding of MUC16 to MSLN. This phenomenon has been reported (U.S. Pat. No. 8,911,732 B2). These results were consistent with the binning data in section 2.1.3 in that clones which bound to three different regions of MSLN showed three different behaviours in the ligand blocking assay.

In conclusion, the results show that a panel of clones was selected that bind to 3 different regions (bins) of MSLN; antibodies binding to one region of MSLN block the binding of MUC16 to MSLN, whereas antibodies binding to the other two regions do not.

### 2.3 Specificity

In light of the different areas of MSLN bound by the panel of antibodies, their specificity for binding to MSLN was tested. Specificity of FS28-024, FS28-026, FS28-091, FS28-185 and FS28-256 was tested by ELISA by comparing the binding to MSLN with binding to other molecules involved in cell adhesion such as CEACAM-5, E-Cadherin, Thrombomodulin and EpCAM.

A similar protocol was used as described in section 1.2.1 in which maxisorp plates were coated with 1 μg/ml of recombinant human MSLN-His-Avi, human CEACAM-5-His-Fc (Sino Biological, 1077-H03H), human E-Cadherin (R&D systems, 8505-EC), human Thrombomodulin (Peprotech, 100-58) or human EpCAM-hFc (in-house production). Binding of the anti-MSLN mAbs, tested at a concentration range of 0.02 to 1000 nM (3-fold dilutions) was detected using anti-human Fab-HRP (Sigma, A0293). Positive controls for the respective molecules included human EpCAM antibody (clone 2G8 from U.S. Pat. No. 8,236,308 B2) CEACAM-5 antibody (clone hMN15 from U.S. Pat. No. 8,771,690 B2), human E-Cadherin antibody, mouse IgG2b clone 180215 (R&D systems, MAB1838), human Thrombomodulin antibody, mouse IgG1, clone 501733 (R&D systems, MAB3947). The latter two were detected using goat anti mouse Fc-HRP (Sigma, A9309) as secondary mAb.

FS28-024, FS28-026, FS28-091 and FS28-185 bound to human MSLN-His-Avi (EC₅₀ around 0.5 nM and maximum binding signal of 3) but no binding was observed to any of the cell adhesion molecules tested up to 1000 nM. Positive control antibodies bound to their respective targets, as expected. Thus, the anti-MSLN antibodies showed a high level of specificity.

### 2.4 Cell Binding

The panel of five selected anti-mesothelin mAbs was analysed for binding to endogenous cell surface MSLN on the human lung cancer cell line NCI-H226.

Briefly, NCI-H226 cells (ATCC CRL-5826) were harvested from T175 cell culture flasks using StemPro Accutase (Gibco, A11105-01). Cells were centrifuged at 1200 rpm for 3 min and resuspended in ice cold FACS buffer made up of DPBS (Life Technologies, 14190169) and 1% BSA (Sigma-Aldrich, A7906) at 2×10⁶ cells/ml and 50 μl per well was seeded in a 96-well V-bottom plate (Costar, 3894). All mAbs tested were diluted in FACS buffer in 120 μl at a concentration range of 0.01-200 nM (4-fold dilutions). The NCI-H226 cells were then centrifuged, supernatant removed and cells resuspended in 100 μl of each mAb dilution and incubated at 4° C. for 45 min. Cells were washed twice by centrifugation with 150 μl FACS buffer, resuspended in 100 μl containing goat anti-human IgG (γ-chain specific) F(ab′)2 fragment-R-Phycoerythrin antibody (Sigma, P8047) diluted 1:1000 in FACS buffer and incubated at 4° C. for 45 min. The cells were washed once with 150 μl FACS buffer and then with 150 μl DPBS, resuspended in 150 μl DPBS containing DAPI (Biotium, 40043) at 1:10.000 and read on the BDCantoll or iQue (Intellicyt). Data was analysed using FlowJo v10 to determine the signal geometric mean for PE for live cells in each well.

The cell binding data (Table 3) showed that FS28-024, FS28-026 and FS28-091 all bound to cell surface cell surface MSLN on NCI-H226 with EC₅₀ in a range of 0.62 to 1.22 nM, as did the positive control G1-AA/SS1. In comparison, FS28-185 and FS28-256 demonstrated weak binding with EC₅₀ greater than 30 nM and a low maximum binding signal (Eₘₐₓ). A representative binding assay is shown in FIG. 1.

TABLE 3
Cell binding of naïve anti-mesothelin
mAbs to NCI-H226 cancer cells

| | |
|---|---|
| mAb (in | Cell binding to NCI-H226 (n = 3) |

| G1-AA format) | EC₅₀ (nM) | Eₘₐₓ (MFI signal) |
|---|---|---|
| FS28-024 | 0.62 | 14647 |
| FS28-026 | 1.08 | 13650 |
| FS28-091 | 1.22 | 11926 |
| FS28-185 | 37.47 | 2435 |
| FS28-256 | 33.13 | 4246 |
| SS1 | 0.90 | 19385 |

### 2.5 Summary of Naïve Screening Procedure

From the 156 mAbs identified by the initial screen of the naïve phage libraries, five anti-human MSLN mAb clones (FS28-024, FS28-026, FS28-091, FS28-185 and FS28-256) were selected based on a set of screening assays that first confirmed binding to full-length, deglycosylated recombinant MSLN as well as the ability to bind to cyno MSLN. Secondly, clones were grouped based on diversity of the region of MSLN they bound (bins) and MUC16 blocking activity and from within these groups, the highest affinity binders were selected. The resulting panel of mAb clones FS28-024, FS28-026, FS28-091, FS28-185 and FS28-256 bound three different regions of MSLN, one of which (bin 1, including FS28-024, FS28-026 and FS28-091) blocks the binding of MUC16 to MSLN in vitro. The panel of five anti-MSLN mAbs showed specific binding to MSLN, different affinities for recombinant and cell-surface MSLN and were selected for further characterisation and/or optimisation as described in Examples 3 and 4 below.

### Example 3: Affinity Maturation and Sequence Optimisation of Naïve Anti-MSLN mAbs

### 3.1 Affinity Maturation of FS28-185 and FS28-256

In comparison to FS28-024, FS28-026 and FS28-091, FS28-185 and FS28-256 had weaker affinity for both recombinant and cell surface MSLN and were therefore subjected to affinity maturation.

### 3.1.1 Affinity Maturation and Screening of FS28-185 and FS28-256

The VH and VL CDR3 regions were affinity matured in parallel in scFv format by randomising overlapping cassettes of five to six amino acids using NNK primers. The regions randomised for FS28-185 were VH G95-M100F and VL S91-A95 and for FS28-256 they were VH Y95-L100B and VL S91-196 (Kabat numbering). Before library generation, parsimonious mutagenesis was performed on potential methionine oxidation and deamidation sites in the CDR1 and CDR3 regions (except for the FS28-256 VL CDR3 library). Phagemid libraries were generated independently and pooled to produce one VH CDR3 and one VL CDR3 library for each clone. Two rounds of selections were performed as described for the naïve campaigns, using 20 nM biotinylated human MSLN-His-Avi in round 1 and either 20 or 2 nM cyno MSLN-His-Avi in round 2. Soluble scFv (single point concentration) were then tested for binding to an ovarian cancer cell line OVCAR-3 (ATCC® HTB-161™). OVCAR-3 cells were harvested using StemPro Accutase (Gibco, A11105-01), centrifuged at 1200 rpm for 3 min and resuspended in FACS buffer (DPBS containing 2% BSA) at 2×10⁶ cells/ml. 100 μl of OVCAR-3 cells were added to 96-well V-bottom plates. Plates were centrifuged at 1200 rpm for 3 mins and the buffer was discarded. 150 μl of scFv was added to the cells and incubated at 4° C. for one hour. ScFvs of parental clones FS28-185 and 256 were included as controls. After washing, cells were resuspended in 100 μl of Penta His Alexa-Fluor 647 (Qiagen, 109-546-098), and washed before being resuspended in 100 μl DPBS containing Sytox Green Nucleic Acid Stain (Invitrogen S7020, 1:10000 dilution). Samples were run on the iQue (Intellicyt Corporation, IQue Plus) and the geometric mean for APC was recorded.

For both FS28-185 and FS28-256, affinity matured clones with improved binding to OVCAR-3 cells were identified. On the basis of cell binding (MFI greater than 850) and sequence diversity, 10 clones were selected from the FS28-256 VH CDR3 and 9 from the VL CDR3 selections. Of the 38 FS28-185 affinity matured clones tested in this assay, 14 were selected from the VH CDR3 selections and one from the VL CDR3 selections. Selected clones were further characterised in a mAb² bispecific antibody format.

3.1.2 Generation of FS28-185 and FS28-256 Based mAb²

For further characterisation of the anti-MSLN binders, the affinity matured VH or VL region of FS28-185 or FS28-256, as well as the parental clones, were produced in mAb² format. The resulting mAb² are IgG1 antibodies comprising of the CDRs of either FS28-185 or FS28-256 clones or the affinity matured variants derived from them, the LALA mutation in the CH2 domain, and a human CD137 receptor-binding site in the CH3 domain. These mAb² molecules were designated FS22-053-008-AA/FS28-185 (SEQ ID NO: 154 and 195) and FS22-053-008-AA/FS28-256 (SEQ ID NO: 156 and 77) and FS22-053-008-AA/FS28-256-x for the affinity matured progeny. The mAb² were produced by transient expression in HEK293-6E cells and, where indicated, purified using mAb Select SuRe protein A columns.

### 3.1.3 Affinity Screening of FS28-185 and FS28-256 Affinity Matured Clones to Immobilised MSLN

The binding of the mAb² containing affinity matured CDR regions were next screened for binding to immobilised human and cyno MSLN-His-Avi by Biacore. HEK293-6E supernatants containing FS22-053-008-AA/FS28-185 and FS22-053-008-AA/FS28-256 affinity matured clones were analysed for binding to immobilised MSLN at 200 RU as described in section 2.1.2. Two concentrations of mAb² were tested, i.e. 50 and 100 nM, and the binding was compared to the binding of the parental antibody also in mAb² format. Binders were ranked and the best six cyno cross-reactive binders of each lineage were re-expressed, purified and tested in cell binding assays as described in section 3.1.4 below. For the FS28-256 lineage, clones with improved VH CDR3 were then shuffled with clones with improved VL CDR3s, creating an additional nine VH/VL pairings, which were produced as mAb² and were also tested.

### 3.1.4 Cell Binding in the Presence of Soluble MSLN

As previously discussed in section 1, soluble MSLN can act as a decoy for the binding of any anti-MSLN antibody. The affinity matured clones were therefore screened for binding to recombinant and cell surface MSLN in the presence and absence of soluble human MSLN. Since soluble, shed MSLN lacks either 7 or 13 C-terminal residues (Zhang et al., 2011), a commercially available human MSLN antigen, MSLN-His Acro, which lacks 18 C-terminal amino acids was used as a mimic. This antigen was used at a 20 nM concentration, which is about 10-20 times the level of soluble MSLN found to be of diagnostic value for defining malignant mesothelioma and lung cancer patients as MSLN positive (Cui et al., 2014).

Cell binding assays using OVCAR-3 cells were performed similarly to the assays described in section 2.4. The effect of the presence of soluble MSLN was tested by pre-incubating the antibodies in the presence or absence of soluble MSLN before mixing with cells and determining if the pre-incubation affected binding to MSLN on the cell surface. The affinity matured clones and parental molecules (all in mAb² format as previously described) were diluted in FACS buffer to give a 2× final concentration in a 96-well V-bottom plate. 60 μl from each well was then added to either FACS buffer alone or to FACS buffer containing 60 μl of 40 nM recombinant hMSLN (R&D systems, 3265-MS-050) (to give a final concentration of 20 nM hMSLN) and pre-incubated at room temperature for 1 hour before 100 μl was added to the cells. Bound antibody was detected using goat anti-human anti-Fcγ Alexa-Fluor 488 (Jackson Immunoresearch, 109-546-098).

For the FS28-185 lineage, all affinity matured clones showed improved cell binding to OVCAR-3 (about 7-fold in ECSO) in comparison to binding by the parental clone. This binding was however reduced in the presence of 20 nM soluble MSLN, resulting in a 3 to 7-fold higher EC₅₀ (from 1.6-1.8 nM to approximately 5-7.8 nM). As for the FS28-256 lineage, five clones were selected as representatives of that lineage, all of which had a range of EC₅₀ varying from 0.9 to 8.6 nM. Most importantly, the binding affinity was retained and there was less than a 2-fold change in binding in the presence of 20 nM soluble MSLN compared to when it was absent. From this data, the clones selected for further testing included FS28-256-012, FS28-256-021, FS28-256-023 and FS28-256-024.

### 3.2 Affinity Maturation of Clone FS28-024 Using NNK Walk Strategy

Whereas FS28-024 bound to human MSLN with a subnanomolar affinity, its affinity for cyno MSLN was about 5-fold lower (see Example 4, Table 4). To improve binding to cyno MSLN, an NNK walk strategy on five residues in the VH CDR3 region was used.

The sequence of the FS28-024 VH and VL was optimised in the same mAb² format as described in section 3.1.2. Parsimonious mutagenesis libraries were generated by diversifying one amino acid residue at a time of the RATLF residues (kabat numbering 95-99) in the VH CDR3, leading to a total of five individual libraries. The libraries were made with low redundancy NNK codons to represent all possible amino acids in the position of interest. Forward and reverse primers were designed according to the guidelines of Quickchange Lightning Site-Directed Mutagenesis Kit (Agilent, 200518), which was used to create the libraries. Each mutant was expressed in small scale in HEK293-6E cells and supernatants were screened by BIAcore for retained or improved binding to human and cyno MSLN-His-Avi. Of the 84 clones screened, few retained binding, most of them being substitutes of T98 residue. Four clones, FS28-024-51, FS28-024-52, FS28-024-53 and FS28-024-060 were re-expressed, purified and their affinities for human and cyno MSLN determined. Only one clone, FS28-024-53 showed an improvement in cyno cross-reactivity which was achieved by a single T98V mutation (Kabat numbering, see Table 4, Example 4). All four clones were taken forward as they might provide alternative sequences and characteristics depending on the application.

### 3.3 Summary of Affinity Maturation

Overall, the affinity maturation and sequence optimisation strategies for the FS28-024, FS28-185 and FS28-256 lineages were successful by further expanding the panel and diversity of anti-MSLN mAbs.

### Example 4: Characterisation of Anti-MSLN Clones

### 4.1 Affinities

Binding of the selected anti-MSLN clones in mAb² format, to recombinant human and cyno MSLN-His-Avi antigen was measured by SPR using a Biacore T200 processing unit (GE Healthcare). As previously described, it was desirable to bind more strongly to immobilised antigen than to soluble antigen. To assess the binding properties of the clones, binding kinetics to immobilised MSLN antigen were determined as described in section 2.1.2 and compared to the kinetics obtained when MSLN antigens in solution bound to captured clones.

For the capture experiments, clones in mAb² format were captured using a BIAcore sensor series S chip Protein G (GE Healthcare, 29179315). mAb² diluted in HBS-EP buffer (GE Healthcare, BR100188) containing 900 mM NaCl₂ at 1 μg/ml, were injected individually on flow cell 2, 3 and 4 at 30 μl/min to achieve a response of approximately 100 RU. G1-AA/HeID1.3 was captured on flow cell 1. The recombinant human and cyno MSLN-His-Avi (section 1.1), diluted in HBS-EP buffer containing 900 mM NaCl₂, were injected on flow cell 1, 2, 3 or 4 as appropriate at a concentration range of 1000 nM to 0.051 nM with 3-fold dilutions for 5 minutes at 70 μl/min and then allowed to dissociate in buffer for 5 minutes. Regeneration was achieved by injecting 10 mM glycine pH1.5 (GE Healthcare, BR100354) and surfactant P20 (GE Healthcare, BR-1000-54) for 20 seconds at a rate of 30 μl/min. Subtracted data (flow cell 2−flow cell 1, flow cell 3−flow cell 1, or flow cell 4−flow cell 1) were analysed using BIAevaluation 3.2 Software (GE Healthcare) to identify binding using the model 1:1 binding, local Rmax and with refractive index (RI) constant 0.

TABLE 4
Affinities to immobilised and in-solution human
mesothelin as well as cyno crossreactivity

| | Affinity to | Affinity to | Ratio of K_{D} | Affinity |
|---|---|---|---|---|
| Clone (in | immobilised | human | in solution/ | to cyno |
| FS22- | human | MSLN-His-Avi | immobilised for | MSLN-His-Avi |
| 053-008-AA | MSLN-His-Avi | in solution | human | in solution |
| mAb² format) | K_{D} (nM) | K_{D} (nM) | MSLN-His-Avi | K_{D} (nM) |
| FS28-024 | 0.39 | 46.5 | 119.2 | 240.0 |
| FS28-024-51 | 0.31 | 47.5 | 154.2 | 148.1 |
| FS28-024-52 | 0.36 | 33.1 | 92.3 | 185.4 |
| FS28-024-53 | 0.89 | 49.2 | 55.3 | 78.3 |
| FS28-024-060 | 0.07 | 5.2 | 78.6 | 265.9 |
| FS28-026 | 0.14 | 700.2 | 5001.4 | 886.0 |
| FS28-091 | 1.1 | 614.9 | 580.1 | >1000 |
| FS28-185 | 30.1 | 452.4 | 15 | 361.5 |
| FS28-256 | 23.7 | 888.8 | 37.5 | 737.9 |
| FS28-256-012 | 4.1 | 861.1 | 209 | 716.7 |
| FS28-256-021 | 3.3 | 23.2 | 7.0 | 16.2 |
| FS28-256-023 | 3.3 | 68.6 | 20.8 | 32.6 |
| FS28-256-024 | 6.7 | 95.4 | 14.3 | 56.7 |
| FS28-256-026 | 3.2 | 62.3 | 19.5 | 33.2 |
| FS28-256-027 | 1.1 | 6.0 | 5.3 | 0.7 |

*Affinity of FS28-026 to 100 RU immobilised MSLN is likely overestimated as the on-rate measurements are outside the limits of the Biacore T200.

The kinetic data demonstrated that all clones tested, with the exception of FS28-024-060, were crossreactive to cyno MSLN-His-Avi, having affinity to the cyno antigen within 5-fold of that to human MSLN-His-Avi.

In addition, for each group of clones that bound to a different MSLN binding region, clones were identified that bound with low nanomolar affinity to immobilised human MSLN-His-Avi. Progeny clones of FS28-256 had a higher affinity than their respective parental antibodies, confirming that the affinity maturation of these clones had been successful. Of interest, all clones had a higher affinity for immobilised MSLN than for in-solution human MSLN-His-Avi, a characteristic that was quantified by calculating the fold difference of the in-solution K_{D} versus the immobilised K_{D} (see Table 4). It can therefore be hypothesised that the anti-MSLN antibodies are not binding with high affinity to the target, as observed by the low affinity for binding to soluble MSLN, but are thought to be binding more strongly to immobilised MSLN due to enhanced avidity interactions with the immobilised antigen. The avidity appears to be antibody specific.

### 4.2 Cell Binding to NCI-H226 in the Presence of Soluble MSLN

All selected clones, supplemented with other non-shuffled FS28-256 affinity matured clones (see Table 5), were then tested for binding to cell surface MSLN on NCI-H226 cells in the absence and presence of 20 nM MSLN. The method used was exactly as described in section 3.1.4. Both EC₅₀ and Eₘₐₓ values were determined (Table 5).

TABLE 5
Cell binding in the absence and presence of soluble mesothelin

| Clone (in | | | | |
|---|---|---|---|---|
| CD137 | NCI-H226 | | NCI-H226 + 20 nM soluble | |
| mAb² format) | EC₅₀ (nM) | Eₘₐₓ (MFI) | EC₅₀ (nM) | Eₘₐₓ (MFI) |
| FS28-024 | 1.47 | 980955 | 5.04 | 1023695 |
| FS28-024-051 | 0.78 | 800589 | 7.47 | 1100503 |
| FS28-024-052 | 1.32 | 957685 | 4.28 | 985916 |
| FS28-024-053 | 1.19 | 914344 | 7.52 | 964941 |
| FS28-024-060 | 1.46 | 1011600 | 25.69 | 1225277 |
| FS28-026 | 1.91 | 729876 | 2.65 | 701999 |
| FS28-091 | 2.72 | 739899 | 3.97 | 744939 |
| FS28-185* | 16.3 | 5155 | 14.3 | 5031 |
| FS28-256 | 15.78 | 275841 | 39.41 | 351430 |
| FS28-256-001 | 4.98 | 703342 | 5.36 | 608248 |
| FS28-256-005 | 5.30 | 759574 | 3.91 | 653731 |
| FS28-256-012 | 8.48 | 968727 | 4.83 | 790055 |
| FS28-256-014 | 3.11 | 771117 | 2.72 | 616427 |
| FS28-256-018 | 6.98 | 637266 | 4.60 | 493728 |
| FS28-256-021 | 3.29 | 919116 | 7.65 | 838717 |
| FS28-256-023 | 3.29 | 856041 | 3.90 | 716321 |
| FS28-256-024 | 3.88 | 812760 | 3.91 | 668584 |
| FS28-256-026 | 2.43 | 741080 | 4.59 | 714886 |
| FS28-256-027 | 3.93 | 986870 | 21.82 | 964426 |

*FS28-185 data were obtained on the BD Cantoll instead of iQue (Intellicyt) resulting in lower. Eₘₐₓ values due to differences in PMT voltages between machines.

The data showed that the anti-MSLN binding Fab arms of the mAb² tested bound with varying affinities to NCI-H226 cells ranging from 0.78 to greater than 16 nM, with the majority of clones exhibiting low nanomolar cell binding affinities, consistent with the affinities reported for the recombinant immobilised MSLN. The ranking of cell binding affinities of the naïve FS28-024, FS28-026, FS28-091, FS28-185 and FS28-256 was also consistent with the ranking data obtained in mAb format (Table 3, section 2.4). Of interest, the effect of 20 nM recombinant MSLN on cell binding affinity was low with minimal (less than 2.5-fold) increases in EC₅₀ observed for most clones. In particular, the cell binding affinities of FS28-256 derived clones, such as FS28-256-001, FS28-256-005, FS28-256-012, FS28-256-014, FS28-256-018, FS28-256-023, FS28-256-024 and FS28-256-026 were not affected by the presence of soluble MSLN. This demonstrated that even in the presence of an excess of soluble MSLN, most clones bound preferentially to the membrane bound form of MSLN.

For clones derived from FS28-024, a more variable effect of soluble MSLN was observed with increases in EC₅₀ ranging from 3.4 to 17.6 fold.

Two clones with the highest affinity for recombinant soluble MSLN (see Table 4), i.e. FS28-024-060 and FS28-256-027 (both having single digit nM K_{D} to MSLN-His-Avi in solution), were most affected when binding to cells in the presence of soluble MSLN, indicating that higher affinity binders are more likely to be affected by shed MSLN. As a result, increases in EC₅₀ in the presence of soluble MSLN are less preferred, since it is the final EC₅₀ in the presence of soluble MSLN that is thought to be most reflective of the affinity of the mAb for these tumour cells in the patient. The actual required affinity of the anti-MSLN antibodies is application dependent.

### Example 5: Sequence Optimisation of FS28-256 Affinity Matured Clones

### 5.1 Sequence Optimisation of FS28-256 Affinity Matured Clones

All FS28-256 lineage clones contained a potential N-linked glycosylation site in the VH CDR2 (IMGT numbering N55-X-S57, wherein X is any residue). Moreover, FS28-256-001, FS28-256-021 and FS28-256-023 harboured a potential deamidation site in the VL CDR3 region at position N116-T117 (IMGT numbering). Similar to the procedure described in section 3.2 an NNK walk strategy was employed to identify amino acid substitutions in clone FS28-256-021 which would remove these potential glycosylation and deamidation sites. Initially the VH CDR2 residue N55 and the VL CDR3 N116 residue were mutated in the respective clones, and mutants were screened for retained for optimised binding to human and cyno MSLN. For FS28-256-021, changes to the VL CDR3 N116 residue resulted in a loss of antigen binding. Of the changes in the VH CDR2 at N55, only four mutants (N55A, N55H, N55S, and N55T) retained binding to human MSLN, though their binding was weaker compared with the parental clone.

Since it was not possible to remove the potential deamidation site in the VL CDR3 by mutating the sequence, alternative strategies were adopted. Since some of the other affinity matured clones derived from FS28-256 shared the same heavy chain sequence as FS28-256-021 but had different light chain sequences, these other clones were explored further. Specifically, FS28-256-027 was selected for testing. As previously described, FS28-256-027 had a higher affinity to soluble MSLN than FS28-256-021 (6.0 nM, Table 4), resulting in reduced binding to cell surface expressed MSLN in the presence of soluble MSLN (Table 5) and consequently was not selected as a preferred clone at the time. To explore whether this clone could be optimised for binding to cell surface expressed MSLN, the N55A, N55H, N55S or N55T mutations identified for the FS28-256-021 clone were introduced into the heavy chain of FS28-256-027 and the binding of the 4 resulting clones was measured by SPR, in a similar manner to the protocol used in Example 4.1 but with an RU of approximately 40 instead of 100, for binding to immobilised and in-solution MSLN. The results are shown in Table 6. Introduction of mutation N55T into FS28-256-027 resulted in a clone, FS28-256-274, which had a much weaker affinity for immobilised MSLN than the other clones and was therefore not progressed further. Introduction of mutations N55H and N55S into FS28-256-027, resulted in clones FS28-256-272 and FS28-256-273, respectively, which bound to soluble MSLN either with a higher or comparable affinity as to immobilised MSLN. Consequently, it was considered likely that binding of both of these clones to cell surface expressed MSLN would be negatively impacted by the presence of soluble MSLN. In contrast, introduction of mutation N55A into FS28-256-027 resulted in a clone, FS28-256-271, which showed the highest affinity for immobilised MSLN of the four clones tested and weaker binding to soluble MSLN. These results showed that, surprisingly, the N55A mutation in the VH CDR2 of the parental clone FS28-256-027 reduced the affinity of binding to both immobilised and soluble MSLN such that FS28-258-271 preferentially targeted immobilised MSLN over soluble MSLN. Consistent with other clones, such as FS28-256-021, which bound with a K_{D} of at 10 nM or less to immobilised MSLN and 10 nM or greater K_{D} to soluble MSLN, it is expected that binding of FS28-258-271 to MSLN on cell surfaces will be less impacted by the presence of soluble MSLN.

TABLE 6
Affinities to immobilised and in-solution human mesothelin

| | | Affinity to | Affinity to | Ratio of K_{D} |
|---|---|---|---|---|
| | | immobilised | human | in solution/ |
| Clone (in | Mutation in | human | MSLN-His-Avi | immobilised for |
| CD137 | Heavy Chain | MSLN-His-Avi | in solution | human |
| mAb² format) | CDR2 | K_{D} (nM) | K_{D} (nM) | MSLN-His-Avi |
| FS28-256-027 | — | 4.7 | 3.2 | 0.68 |
| FS28-256-271 | N55A | 5.9 | 18.2 | 3 |
| FS28-256-272 | N55H | 10.7 | 7.6 | 0.7 |
| FS28-256-273 | N55S | 6.0 | 7.4 | 1.2 |
| FS28-256-274 | N55T | 19.8 | 55.9 | 2.8 |

For mAb² FS22-172-003-AA/FS28-256-271, cyno cross-reactivity was determined by SPR using a steady-state kinetic analysis. A CM5 chip (GE Healthcare BR-1005-30) was coated with hMSLN-His-Avi or cMSLN-His-Avi at approximately 50 RU according to manufacturer's instructions. mAb² were injected at a range of concentrations in a three-fold dilution series starting at 243 nM, at a flow rate of 10 μl/min. The association time was 1000 sec to steady state and the dissociation time was 30 sec. Running buffer was HBS-EP (GE Healthcare BR100188). Flow cells were regenerated by injecting Glycine-HCl pH1.5 at a flow rate of 30 μl/min for 30 seconds. Data were analysed by double referencing against a flow cell which was intentionally left blank (no antigen coating). Steady state affinity model was used to analyse kinetic data using the BiaEvaluation software version 3.2. Binding to cyno MSLN was within 3 fold of binding to human MSLN.

### 5.2 Antibody-Dependent Cellular Cytotoxicity (ADCC) Activity

To assess effector function of the MLSN mAbs, the molecules were tested in an ADCC in vitro assay. For this purpose, FS28-256-271 and FS28-024-052 antibodies were produced in human IgG1 format with or without LALA mutations (LALA or G1, respectively).

Raji cells expressing human MSLN (Raji.hMSLN cells) were generated by lentiviral transduction using the Lenti-X HTX Packaging system (Takara, cat. No. 631253). Lenti-X expression vector (pLVX) (Takara, cat. No. 631253) containing cDNA encoding human MLSN was co-transfected with a Lenti-X HTX Packaging Mix into the Lenti-X 293T cell line (Takara, cat. No. 632180) to generate virus. A Raji cell line (ATCC® CCL-86™) was then transduced with these lentiviral vectors. Expression of human MLSN on these cells was confirmed by binding of G1/SS1 positive control antibody for 1 hour and then a fluorescently-labelled anti-human Fc detection antibody (Stratech Scientific Ltd, cat. no. 109-546-098-JIR) was used to detect cell binding.

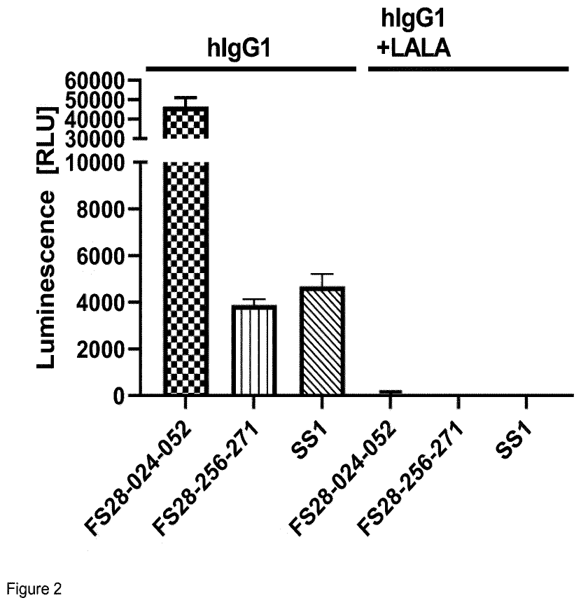

An ADCC reporter bioassay kit (Promega, cat. No. G7010) was used following the manufacturer's protocol. Effector cells from the ADCC kit were mixed in a 20:1 ratio with Raji.hMSLN cells. mAb² or control antibody SS1 were titrated on a 96-well plate and incubated at 37° C. 5% CO₂ for 6 hours. ADCC activity was measured by adding a luciferase substrate from the Bio-Glo assay system kit (Promega, cat. No. G7941) as per the manufacturer's instructions. Luminescence signal was plotted vs the log concentration of antibody and the resulting curves were fitted using the log (agonist) vs response equation in GraphPad Prism. The results are shown in FIG. 2 and demonstrate that both FS28-256-271 and FS28-024-052 were able to elicit ADCC activity when in human IgG1 format. As expected ADCC activity was lost when the LALA mutation was introduced into the hIgG1 backbone of these antibodies. This data demonstrates that the MSLN antibodies described here can be used to elicit effector functions to modulate immune responses.

### 5.3 Potency of FS28-256-271 in Primary T Cell Assays

5.3.1 Potency of FS28-256-271 in an OX40 mAb² Format In Vitro in a Primary T Cell Assay

Pan T cells were isolated from peripheral blood mononuclear cells (PBMCs) obtained from leucocyte depletion cones, a by-product of platelet donations. Briefly, leucocyte cone contents were flushed with PBS and overlaid on a Ficoll gradient (GE Lifesciences cat no 17144002). PBMCs were isolated by centrifugation and recovery of cells that did not cross the Ficoll gradient. PBMCs were further washed with PBS and the remaining red blood cells were lysed through the addition of 10 ml red blood cell lysis buffer (eBioscience) according to the manufacturer's instructions. PBMCs were counted and resuspended to 2.0×10\hat{ }6 cells/ml in T cell medium (RPMI medium (Life Technologies) with 10% FBS (Life Technologies), 1× Penicillin Streptomycin (Life Technologies), Sodium Pyruvate (Gibco), 10 mM Hepes (Gibco), 2 mM L-Glutamine (Gibco) and 50 μM 2-mercaptoethanol (Gibco).

T cells were then isolated from the PBMCs using a Pan T Cell Isolation Kit II (Miltenyi Biotec Ltd) according to the manufacturer's instructions. Human T-Activator CD3/CD28 Dynabeads (Invitrogen 111.32D) were resuspended by vortexing. Beads were washed twice with T cell medium (RPMI medium (Life Technologies) with 10% FBS (Life Technologies), 1× Penicillin Streptomycin (Life Technologies), Sodium Pyruvate (Gibco), 10 mM Hepes (Gibco), 2 mM L-Glutamine (Gibco) and 50 μM 2-mercaptoethanol (Gibco). T cells at a concentration of 1.0×10⁶ cells/ml in T cell medium were stimulated with the washed human T-Activator CD3/CD28 Dynabeads at a 2:1 cell to bead ratio in a T-25 flask (Sigma) and incubated overnight at 37° C., 5% CO₂ to activate the T cells. Activated T cells were washed from the Dynabeads and resuspended in T cell medium at a concentration of 2.0×10⁶ cells/ml. 96-well flat-bottomed plates were coated with anti-human CD3 antibody through incubation with 2.5 μg/ml anti-human CD3 antibody (R&D Systems clone UHCT1) diluted in PBS for 2 hours at 37° C., 5% CO₂ and then washed twice with PBS. Activated T cells were added to the plates at 6×10⁵ cells/well. Mesothelin expressing cells, NCI-H226, were added at 20,000 cells\well. A mAb² comprising an anti-OX40 Fcab as well as the anti-MSLN Fab FS28-256-271 was tested in this assay, together with control antibodies. The assay plates were incubated at 37° C., 5% CO₂ for 72 hours. Supernatants were collected and IL-2 release was measured by Human IL-2 ELISA (Life Technologies, 88-7025-88). The concentration of human IL-2 (hIL-2) was plotted vs the log concentration of antibody and the resulting curves were fitted using the log (agonist) vs response equation in GraphPad Prism. The results are shown in Table 7 and demonstrate that FS28-256-271 in a mAb² format is able to bind to MSLN expressed on a cell surface and crosslink the antibody such that the OX40 binding site can bind to and activate OX40.

5.3.2 Potency of FS28-256-271 in a CD137 mAb² Format in an In Vitro Primary T Cell Assay

Peripheral blood mononuclear cells (PBMCs) were obtained as described in 5.2. CD8+ T cells were isolated using the CD8+ T cell isolation kit II (Miltenyi Biotec Ltd, 130-096-495) according to the manufacturer's instructions. 96-well flat bottom tissue culture plates were coated with 8 μg/ml anti-CD3 antibody (Clone UCHT1, R&D Systems, MAB100-SP) in PBS overnight at 4° C. The plates were then washed 3 times with 200 μl PBS. NCI-H226 cells were plated at 2×10⁴ cells per well on to anti-CD3 antibody-coated (8 μg/ml) 96 well flat bottom plates in 100 μl T cell culture medium (RPMI medium (Life Technologies, 61870-044) with 10% FBS (Life Technologies), 1× Penicillin Streptomycin (Life Technologies, 15140122), 1 mM Sodium Pyruvate (Gibco, 11360-070), 10 mM Hepes (Sigma-Aldrich, H0887), 2 mM L-Glutamine (Sigma-Aldrich, G7513) and 50 μM 2-mercaptoethanol (Gibco, M6250)). Once cells had adhered after 4 hours incubation, all T cell culture medium was removed and replaced with 50 μl T cell culture medium containing T cells at a concentration of 4.0×10¹ cells/ml resulting in 2.0×10⁴ cells/well. Control antibodies or a mAb² comprising a CD137 Fcab and the MSLN Fab FS28-256-271 were diluted in T cell medium at a 4× final concentration starting at 60 nM and 1:3 or 1:7 serial dilutions were carried out. 50 μl of antibody titration was added to the cells for a total assay volume of 200 μl and 1× concentration of antibody. The assay was incubated at 37° C., 5% CO₂ for 72 hours. Supernatants were collected and assayed with a V-PLEX IL-2 kit from Meso Scale Discovery (K151QQD-4) following the manufacturer's instructions. The concentration of human IL-2 (hIL-2) was plotted vs the log concentration of antibody and the resulting curves were fitted using the log (agonist) vs response equation in GraphPad Prism. The results are shown in Table 7 and demonstrate that FS28-256-271 in a mAb² format is able to bind to MSLN on the cell surface and crosslink the antibody such that the CD137 binding site can bind to and activate CD137.

### 5.3.3 Summary of Sequence Optimised MSLN Clone FS28-256-271 and its Use to Drive Agonism of TNFRSF Members.

Table 7 shows the results of the T cell assays described in 5.3.1 and 5.3.2 which demonstrate the ability of anti-MSLN Fabs such as FS28-256-271 in a mAb² format to crosslink and activate different TNFSFRs such as OX40- or CD137 to induce agonism in T cells.

TABLE 7
T cell activation assay using FS28-256-271 as mesothelin-binding
Fab in a mAb² format, and using NCI-H226 cells for crosslinking

| Clone | EC₅₀ (nM) | Emax (pg/ml IL-2) |
|---|---|---|
| OX40/FS28-256-271 mAb² | 0.11 | 6509 |
| CD137/FS28-256-271 mAb² | 0.096 | 4871 |

Summary of Isolation Anti-Human Mesothelin mAbs (Examples 1 to 5)

Phage selection and antibody screening strategies aimed at isolating anti-human MSLN antibodies that bind to cell surface MSLN, led to the identification of a panel of specific anti-mesothelin binding antibodies with a range of affinities, MUC16-MSLN blocking activities, MSLN region binding bins and cell binding characteristics. Due to the screening cascade performed, most anti-MSLN binding Fabs, whether tested in mAb or mAb² format, preferentially bound to cell surface MSLN.

### Example 6: Selection and Characterisation of Anti-Mouse MSLN Antibodies

6.1 Naïve Selection of Anti-Mouse MSLN mAbs

The amino acid identity between mouse and human MSLN is low (60%). To enable in vivo Proof of Concept (PoC) studies in mice, the inventors set out to isolate anti-mouse MSLN mAbs with similar properties as the anti-human MSLN mAbs described in Example 1 to 5.

Phage selections, using the synthetic naive phagemid libraries displaying the Fab domain of human IgG1 germlines with randomisation in the CDR1, CDR2 and CDR3 (MSM Technologies) were used for selections with biotinylated mouse MSLN-His-Avi (SEQ ID NO 171, see section 1.1) as described in section 1.2. Four rounds of selections were performed with decreasing concentrations of biotinylated mMSLN-His-Avi and similarly to the anti-human MSLN selections, epitope masking strategies were performed in a subsequent campaign. In addition, after a first round of using recombinant antigen, HEK293-mMSLN cells were generated and used in round 2, 3 and 4.

Briefly, mouse MSLN sequence was subcloned into the pcDNA5/FRT/TO vector (Life technologies, V652020) and then co-transfected with the Flp recombinase expression plasmid, pOG44 (Life Technologies, V600520) into Flp-In TREx 293 cell lines (Life Technologies, R78007). Cells were grown in DMEM containing 10% FBS, 100 μg/ml Hygromycin B (Melford Laboratories Ltd, Z2475) and 15 μg/ml Blasticidin (Melford Laboratories Ltd, B1105) for 3-4 weeks until colonies of stably transformed cells had formed. These colonies were amplified in the presence of 1 μg/ml Doxycyclin (Sigma Aldrich, D9891) and tested for expression of MSLN using anti-mouse MSLN (LS Bio, LS-C179484).

In total 47 individual mAbs from enriched populations were screened for antigen binding and 45 unique positive binders were subcloned and expressed as soluble mAbs in IgG1 LALA format as previously described in Example 1.3. mAbs were characterised for specific binding to immobilised mMSLN-His-Avi by ELISA and ranked based on affinity to about 50 or 200 RU of immobilized mMSLN-His-Avi in kinetic experiments using Biacore analysis. This identified a panel of mAbs, including FS28m-194, FS28m-201, FS28m-209, FS28m-216, FS28m-228, FS28m-261 and FS28m-265 with affinities ranging from 1 to 25 nM. In addition, binding to different regions of MSLN was tested as described in section 2.1.3. A mouse cross-reactive mAb G1-AA/MOR6626, generated by cloning the VH and VL of MOR6626 clone (Patent publication no WO 2009/068204 A1) was used as a positive control. Most clones, amongst which FS28m-228 failed to bind to MSLN that was already bound to MOR6626, whereas others like FS28-194 or FS28-026 showed partial or full binding respectively. Thus, clones binding to different regions (bins) were isolated. The anti-MSLN binding regions of MOR6626 have been used for in vivo PoC studies (Patent publication no. US 2017/0342169 A1). Based on the obtained data, FS28m-228 might bind to a similar region on MSLN as MOR6626.

6.2 Affinity Maturation of Anti-Mouse MSLN mAbs

The VH and VL CDR3 regions of FS28m-228 were optimised in parallel in scFv format by randomising overlapping cassettes of five amino acids using NNK primers as described in example 3.1.1. Two rounds of selections were performed using 50 nM biotinylated mMSLN-His-Avi in round 1 and 0.2 nM mMSLN-His-Avi in round 2. For the second round, an off-rate selection pressure was also applied by adding a 1000-fold excess (200 nM) of mMSLN-His-Avi (non-biotinylated) and incubating the antigen/phage mixture for 2.5 hours at room temperature. Soluble scFv (single point concentration) were then tested for binding to mMSLN-His-Avi using Octet. Briefly, streptavidin sensors (ForteBio, 18-5019) were incubated with mMSLN-His-Avi (10 μg/ml) for 5 min. Association of soluble scFv, diluted with 10× kinetic buffer to final concentration of 1× buffer (ForteBio, 18-1092), to mMSLN was analysed for 5 min, followed by a dissociation step of 5 min. Compared to the parental FS28m-228 scFv, about 66 out of 85 clones tested showed improved binding. Nine clones were taken forward for expression into mAb² format, combining the Fab arms with an Fcab which binds to mouse CD137, called FS22-063-AA, using the same cloning and expression method as described (section 3.1.2). The resulting mAb² designated were tested for their MSLN binding affinities.

6.3 Binding Affinity of Anti-Mouse MSLN mAb² to Immobilised and Soluble Mouse MSLN

Like the anti-human MSLN binders, the affinity matured variants derived from FS28m-228 were tested for binding to immobilised and soluble MSLN using the Biacore. The procedure for binding to immobilised MSLN was similar to the method described in section 2.1.4 with mMSLN-His-Avi immobilised at 50 RU. To determine the affinity for soluble MSLN the mAb² were captured via anti-human Fc. Briefly, 25 μg/ml anti-human IgG (Fc) antibody (GE Healthcare, Human Antibody Capture Kit, BR100839) was coated on flow cells 1, 2, 3 and 4 of a Biacore sensor chip CM5 (GE Healthcare, BR100530) achieving a final response of approximately 750 RU. The mAb² clones, diluted in HBS-EP buffer (GE Healthcare, BR100188) at 50 nM, were injected individually on flows cell 2, 3 and 4 at 30 μl/min to achieve a response of approximately 100 RU. The recombinant mMSLN-His-Avi antigens, diluted in HBS-EP buffer, were injected on flow cell 1, 2, 3 or 4 as appropriate at a concentration range of 243 nM to 0.11 nM with 3-fold dilutions for 5 minutes at 70 μl/min and then allowed to dissociate in buffer for 5 minutes. Regeneration was achieved by injecting 3 M magnesium chloride (GE Healthcare, Human Antibody Capture Kit, BR100839) for 30 seconds at a rate of 30 μl/min.

The kinetic data of both the immobilised and in-solution affinities (Table 8) showed that in comparison to the parental FS28m-228 Fab arm, the affinity matured clones showed improved binding to mouse MSLN-His-Avi antigen. Moreover, all clones showed binding kinetics with an affinity to immobilised which was higher than to soluble MSLN, with varying ratios. Like the human clones described in sections 4.1 and 5.1, it was thought that these clones would have stronger binding to membrane bound than soluble shed MSLN due to enhanced avid binding interactions. Thus, the affinity maturation of FS28m-228 led to a panel of affinity matured clones that had increased affinity towards mouse MSLN and all bound more strongly to immobilised MSLN than soluble MSLN. Clone FS28m-228-010 was selected as the preferred clone, as it had the highest affinity to immobilised mouse MSLN and low affinity to in-solution mouse MSLN.

TABLE 8
Affinities to immobilised and in-solution human mesothelin

| | Affinity to | Affinity to | Ratio of K_{D} |
|---|---|---|---|
| Clone (in | immobilised | in-solution | in solution/ |
| FS22-063AA | mMSLN-His-Avi | mMSLN-His-Avi | immobilised for mouse |
| mAb² format) | K_{D} (nM) | K_{D} (nM) | MSLN-His-Avi |
| FS28m-228 | 7.90 | 252 | 31.9 |
| FS28m-228-010 | 2.6 | 60.24 | 23.2 |

6.4 Summary of Isolation Anti-Mouse Mesothelin mAbs

Phage selection and antibody screening strategies led to the identification of a panel of anti-mouse mesothelin binding clones with a range of affinities and which bind to different regions of mMSLN. Like the anti-human MSLN binders, the clones showed binding characteristics favouring binding to immobilised mMSLN than soluble mMSLN rendering them suitable molecules for studying in murine in vivo PoC studies.

6.5 In Vivo Efficacy of mAb² Containing the Anti-Mouse MSLN mAb FS28m-228-010

Having shown that anti-MLSN mAbs in hIgG1 isotype were able to elicit ADCC activity, it was desirable to demonstrate that the anti-MLSN mAbs were able to drive MLSN-dependent crosslinking in vivo in a CD137 mAb² format.

A syngeneic mouse tumour model expressing mouse MSLN was constructed. CT26 colon carcinoma cells (ATCC, CRL-2638) expressing full-length mouse mesothelin (SEQ ID NO: 171), were produced by lipofection (Lipofectamine 3000, Thermo Fisher Scientific, catalogue number L3000008) using the pcDNA3.1 vector (+) (Thermo Fisher Scientific, catalogue number V79020). Following the manufacturer's protocol, the CT26 cells were transfected with the pcDNA3.1 vectors containing the mouse MSLN cDNA. A stable transfection was achieved using geneticin as the selection antibiotic (at 600 μg/ml) in complete media (RPMI, 10% FBS). Expression of mouse MSLN on the CT26 cells was confirmed by flow cytometry by using the positive control antibody MOR6626 (WO 2009/068204 A1). Specifically, cells were incubated with the positive control antibody for 1 hour and then a fluorescently-labelled anti-human IgG detection antibody (Stratech Scientific Ltd, catalogue no. 109-546-098-JIR) was used to detect cell binding. Clonal populations were expanded and subsequently analysed to determine the relative expression levels using the same flow cytometric procedure, after which one clone was selected and denominated CT26.G10.

CT26.G10 tumour growth was confirmed in vivo. Balb/c female mice (Charles River) aged 8-10 weeks were micro-chipped and given a unique identifier. Each cohort had 17 mice and each animal received 1×10¹ cells injected subcutaneously in the dorsal left flank in 100 μl serum free medium. Tumour volume measurements were taken three times per week with calipers to determine the longest axis and the shortest axis of the longest axis and the shortest axis of the tumour. The following formula was used to calculate the tumour volume:

$$\begin{matrix}Volume = L \times S 22 \\ Where L = longest axis ; S = shortest axis\end{matrix}$$

The trial was halted when tumour volume reached the humane endpoint in accordance with the United Kingdom Animal (Scientific Procedures) Act and EU Directive EU86/609. Tumour tissues were collected at the termination of the study, and expression of membrane-bound mesothelin was confirmed by immunohistochemical staining in formalin fixed paraffin embedded (FFPE) tumour tissues as follows: 4 μm FFPE tissue sections were deparaffinised and antigen retrieved using low pH 6.1 at 97° C. (Dako PT Link) followed by a peroxidase block and protein block prior to incubation with a primary anti-mesothelin antibody (LifeSpan Biosciences, catalogue no. LS-C407883) at a concentration of 1 μg/ml. The anti-mesothelin antibody was detected using a labelled polymer-HRP anti-rabbit secondary reagent and a DAB (3,3′-diaminobenzidine) chromogenic endpoint (Dako EnVision+System).

To assess antibody FS28m-228-010, the following molecules or combinations were tested in vivo: FS28m-228-010 antibody in human IgG1 isotype with LALA mutations (G1-AA/FS28m-228-010), two “mock” CD137 mAb² (FS22m-063-AA/HeID1.3 and FS22m-063-AA/4420), a combination of the FS28m-228-010 antibody with a mock CD137 mAb² with LALA mutations (G1-AA/FS28m-228-010+FS22m-063-AA/HeID1.3), a human isotype control antibody (G1-AA/HeID1.3), and finally a CD137/MSLN mAb² (FS22m-063-AA/FS28m-228-010) with LALA mutations (SEQ ID NO: 196 and 197).

Balb/c female mice (Charles River) aged 8-10 weeks and weighing 20-25 g each were acclimatised for one week prior to the study start. All animals were micro-chipped and given a unique identifier. With the exception of FS22m-063-AA/4420 (n=10 mice), each cohort comprised 20 mice. The CT26.G10 colon carcinoma cell line was expanded and cell banks generated. Each animal received 1×10⁵ cells injected subcutaneously in the left flank in 100 μl serum free media. Any mice which did not have tumours 12 days following tumour cell inoculation were removed from the study.

200 μg doses of each antibody (˜10 mg/kg) were prepared and injected intraperitoneally (IP) into mice. In addition, both the G1-AA/FS28m-228-010+FS22m-063-AA/HeID1.3 were each prepared at 200 μg per dose (˜10 mg/kg) for the combination group. 200 μl doses were administered to the mice on days 12, 14 and 16 (q2dx3), following tumour inoculation. Tumour volume measurements were made three times per week using calipers and mice were monitored closely. The study endpoint was determined by humane endpoints based on tumour volume and condition of the mice.

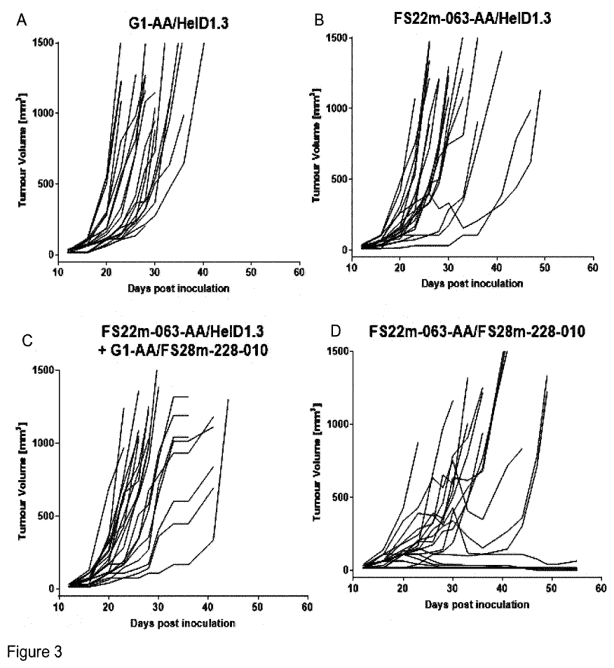

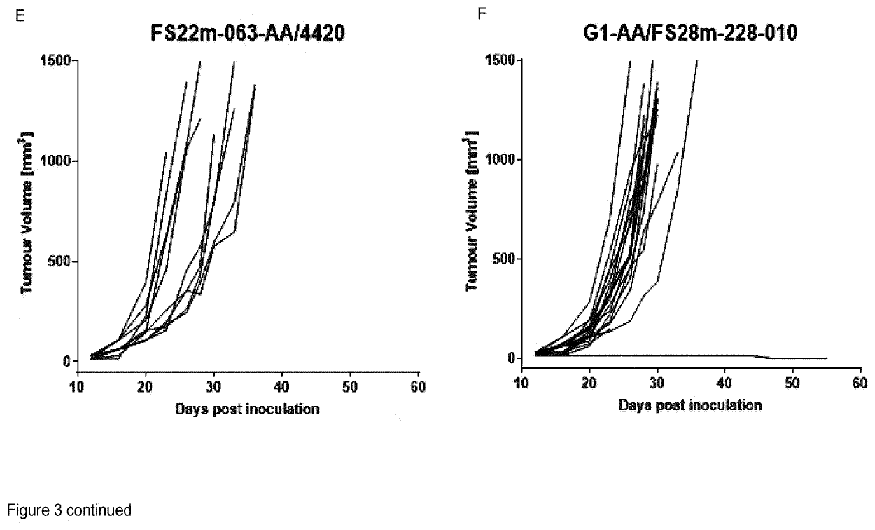

As shown in FIG. 3, the FS22m-063-AA/FS28m-228-010 mAb² significantly inhibited tumour growth compared to the G1-AA/HeID1.3 isotype control. Table 9 shows pairwise comparison of the tumour growth rates for all treatment groups over the full course of the study using Mixed Model analysis, comparing all groups to the G1-AA/HeID1.3 isotype control.

All animals bearing tumours measuring equal or below 62.5 mm³ at the end of the study were counted as fully responding animals (see Table 10). 35% of anti-CD137/MSLN mAb²-treated animals were complete responders to treatment at the end of study, compared to 0% in the G1-AA/HeID1.3 isotype control, FS22m-063-AA/HeID1.3, FS22m-063-AA/4420, and FS22m-063-AA/HeID1.3 and G1-AA/FS28m-228-010 combination groups.

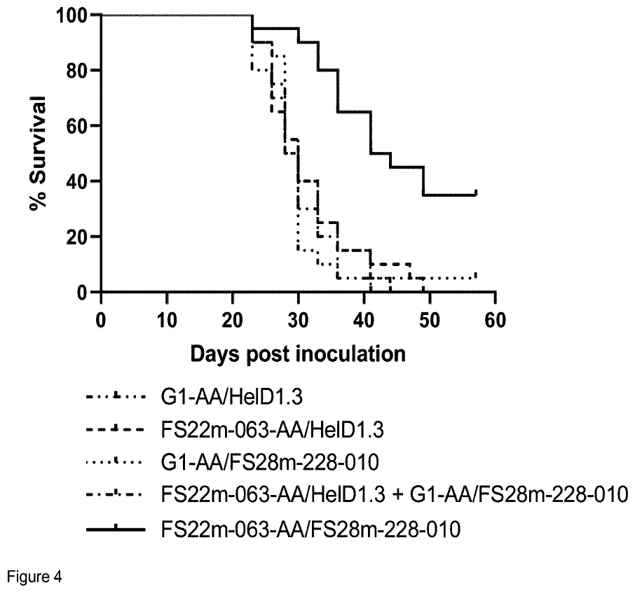

Survival analysis (FIG. 4 and Table 11) showed that the FS22m-063-AA/FS28m-228-010 mAb² induced a significant survival benefit compared with the G1-AA/HeID1.3 antibody, whereas the G1-AA/FS28m-228-010, FS22m-063-AA/HeID1.3 and FS22m-063-AA/4420) did not demonstrate a survival advantage. In addition, the FS22m-063-AA/FS28m-228-010 mAb² resulted in an improved median survival time of 42.5 days compared with G1-AA/HeID1.3 (29 days), FS22m-063-AA/HeID1.3 (30 days), FS22m-063-AA/4420 (29 days), G1-AA/FS28m-228-010 (30 days) and combination of FS22m-063-AA/HeID1.3 with G1-AA/FS28m-228-010 (29 days).

These data suggest that crosslinking of the mAb² via MSLN is capable of driving CD137 agonism in the tumour; an action of the bispecific antibody which is superior to targeting CD137 and/or MSLN alone (and even in combination), resulting in significantly improved survival in tumour-bearing mice. The mAb on its own (G1-AA/FS28m-228-010) with LALA mutation showed no intrinsic activity in this study. Without wishing to be bound by theory, it expected that an effector-competent version of antibody FS28m-228-010 (excluding the LALA mutation) would show ADCC activity and anti-tumour efficacy consistent with the in vitro results observed in section 5.2.

TABLE 9
Results of pairwise comparison of the tumour growth
using Mixed Model analysis, comparing all groups
to the G1-AA/HelD1.3 isotype control.

| Groups | Mixed Model Analysis | |
|---|---|---|
| G1-AA/HelD1.3 10 mg/kg (negative control) | P-value | Significance |
| G1-AA/FS28m-228-010 10 mg/kg | 0.4367 | NS |
| FS22m-063-AA/HelD1.3 10 mg/kg | 0.0017 | *** |
| FS22m-063-AA/4420 10 mg/kg | 0.7067 | NS |
| G1-AA/FS28m-228-010 10 mg/kg + FS22m- | 0.2093 | NS |
| 063-AA/HelD1.3 10 mg/kg | | |
| FS22m-063-AA/FS28m-228-010 10 mg/kg | 0.0000 | **** |

NS—not significant p ≥ 0.05;
**** p-value < 0.0001;
*** p-value 0.0001-0.001

TABLE 10
Number and percentage of tumour-free mice (tumours ≤62.mm³)
by the end of study in the CT26.G10 syngeneic tumour model.

| | |
|---|---|
| | Tumour-free mice |
| Groups | at study end |

| | | |
|---|---|---|
| G1-AA/HelD1.3 10 mg/kg (negative control) | 0/20 | (0%) |
| G1-AA/FS28m-228-010 10 mg/kg | 1/20 | (5%) |
| FS22m-063-AA/HelD1.3 10 mg/kg | 0/20 | (0%) |
| FS22m-063-AA/4420 10 mg/kg | 0/10 | (0%) |
| G1-AA/FS28m-228-010 10 mg/kg + | 0/20 | (0%) |

FS22m-063-AA/HelD1.3 10 mg/kg

| | | |
|---|---|---|
| FS22m-063-AA/FS28m-228-010 10 mg/kg | 7/20 | (35%) |

TABLE 11
Median survival times for animals treated with each
compound, and results of pairwise statistical analyses
(Log-rank) in CT26.G10 syngeneic tumour model

| | Median | Log-rank | |
|---|---|---|---|
| | Survival | | Sig- |
| Groups | (Days) | | nifi- |
| G1-AA/HelD1.3 10 mg/kg | 29 | P-value | cance |
| G1-AA/FS28m-228-010 10 mg/kg | 30 | 0.993 | NS |
| FS22m-063-AA/HelD1.3 10 mg/kg | 30 | 0.3952 | NS |
| FS22m-063-AA/4420 10 mg/kg | 29 | 0.9645 | NS |
| G1-AA/FS28m-228-010 10 mg/kg + | 29 | 0.4706 | NS |
| FS22m-063-AA/HelD1.3 10 mg/kg | | | |
| FS22m-063-AA/FS28m-228-010 10 mg/kg | 42.5 | <0.0001 | **** |

NS—not significant p ≥ 0.05;
**** p-value < 0.0001

6.6 Mechanism of Action of an Anti-Mouse CD137/MSLN mAb² Containing the Anti-Mouse MLSN mAb FS28m-228-010

To further understand the pharmacology of the anti-tumour response observed with FS22m-063-AA/FS28m-228-010 containing the anti-mouse MSLN mAb FS28m-228-010, the mechanism of action of CD137/MSLN mAb² in a MSLN-positive syngeneic tumour model was investigated.

Mice were prepared as described in Example 6.5, and inoculated with the CT26.G10 colon carcinoma cell line. Each cohort consisted of 20 mice. FS22m-063-AA/FS28-228-010 (CD137/MSLN) mAb², human IgG1 isotype control (G1-AA/4420) and an anti-CD137 agonist antibody (clone 3H3; G1/3H3; Rickert et al., 2016) was also included for comparison. All three antibodies were prepared at 134 μg per dose (approximately 6.7 mg/kg in a 20 g mouse) in DPBS+1 mM arginine+0.05% Tween 80 and injected intraperitoneally (IP) into mice. Each mouse received the antibodies by one 200 μl intraperitoneal injection at a fixed dose of 134 μg on day 20 following tumour inoculation. Tumour volume measurements were made three times per week using calipers as described in Example 6.5, and mice were monitored closely.

Six mice per group were necropsied 24, 72, 144 and 192 hours post dose at day 20 following tumour inoculation. Spleen, blood and tumour tissue were taken for analysis from CT26.G10 tumour-bearing mice treated with either FS22m-063-AA/FS28-228-010, G1-AA/4420, or G1/3H3. All samples were investigated for T cell abundance and proliferation by flow cytometry, as T cell activation and proliferation markers are known to be downstream effects of CD137 agonism (Fisher et al., 2012). In addition, serum from blood was also collected for detection and quantification of soluble MSLN expression. Spleen and tumour tissue were disaggregated to single cell suspension by standard mechanical and enzymatic methods, and red blood cells were lysed once in red blood cell lysis buffer (Miltenyi Biotec Ltd., 130-094-183). Blood was collected by terminal cardiac bleed, and half collected into EDTA-containing tubes for single cell analysis by flow cytometry and half of blood was collected into clotting activator/serum tubes for analysis of soluble MSLN. Whole blood collected in EDTA-containing tubes were lysed three times in red blood cell lysis buffer (Miltenyi Biotec Ltd., 130-094-183) according to Manufacturer's instructions. Blood collected in serum tubes was fractionated by centrifugation and serum removed for analysis of soluble MSLN.

Single cells from spleen, tumour and blood were then treated the same, and cells were washed once with PBS and samples stained with fixable viability dye (eBioscience, 65-0865-14). Cells were subsequently stained for cell surface markers with an antibody staining panel shown in Table 12 (all but intracellular markers, Ki67 and FoxP3), in the presence of Fc block (eBioscience, 16-0161-85 at 1:25) for 45 minutes at 4° C. Cells were then fixed and permeabilised with the eBioscience FoxP3 staining kit (eBioscience, 00-5523-00) according to manufacturer's instructions. Cells were resuspended in 100 μl permeabilization buffer with intracellular markers Ki67 and FoxP3 antibodies and incubated overnight at 4° C. in the dark. Prior to acquisition on a BD Fortessa flow cytometer, cells were washed once with permeabilization buffer and resuspended in 120 μl PBS containing 0.5% BSA. Data was acquired using BD FACS Diva software, and analysed with FlowJo (V10), and Microsoft Excel. The data shows the abundance and proliferation of CD8⁺ T cellsat 144 hours following dosing, as a percentage of the parental population.

TABLE 12
Flow Cytometry panel

| Target | Clone | Fluorophore | Manufacturer | Cat No. |
|---|---|---|---|---|
| CD45 | 30-F11 | Alexa700 | eBioscience | 56-0451 |
| CD3e | 145-2C11 | PE-Cy7 | eBioscience | 25-0031-82 |
| CD8 | 53-6.7 | BUV737 | BD Bioscience | 564297 |
| CD4 | RM4-5 | BUV395 | BD Bioscience | 740208 |
| FoxP3 | FJK-16s | PerCP-Cy5.5 | eBioscience | 45-5773 |
| CD49b | DX5 | BV421 | Biolegend | 563063 |
| CD103 | M290 | BV786 | BD Bioscience | 564322 |
| CD137 | 17B5 | APC | eBioscience | 106110 |
| CD69 | H1.2F3 | BV510 | Biolegend | 104505 |
| PD1 | 29F.1A12 | FITC | Biolegend | 135220 |
| Ki67 | SolA15 | PE | eBioscience | 12-5698-82 |
| Viability | N/A | eFluor780 | eBioscience | 65-0865-14 |

As shown in Table 13, an increase in the percentage of CD8⁺ T cells in the tumour was observed at 144 hours following dosing with G1/3H3 and FS22m-063-AA/FS28-228-010 mAb², compared to the control treatment group (G1-AA/4420). The mean percentage of CD8⁺ T cells in the tumour increased from 32.1% (G1-AA/4420) to 56.1% with G1/3H3 and 58.4% with FS22m-063-AA/FS28m-228-010 at 144 hours post dose.

In addition, an increase in the abundance of CD8⁺ T cells was also observed in the blood and spleen, but only with G1/3H3 in comparison to IgG1 control. In the blood at 144 hours post dose, the mean percentage of CD8⁺ T cells increased from 22.6% (G1-AA/4420) to 57.0% (G1/3H3), yet this increase was not observed with FS22m-063-AA/FS28m-228-010 (25.8%). Similarly, in the spleen, the mean percentage of CD8⁺ T cells increased from 28.8% (G1-AA/4420) to 38.0% with G1/3H3, yet this increase was not observed with FS22m-063-AA/FS28m-228-010 (29%).

This suggests that the FS22m-063-AA/FS28m-228-010 mAb² increases CD8⁺ T cells specifically in the tumour, where MSLN is expressed, whereas the CD137-targeting antibody, G1/3H3, also demonstrates peripheral (blood and spleen) increases in CD8⁺ T cells.

To identify whether there were any differences in the proliferation of CD8⁺ T cells following dosing, proliferation marker, Ki67, was analysed on CD8⁺ T cells in tumour, blood and spleen. As shown in Table 14, a high proportion of CD8⁺ T cells expressed Ki67⁺ in the control group (mean expression of 75.1%), suggesting a high level of proliferating CD8+ T cells in the tumour in the CT26.G10 model. This may contribute to the unclear differences in Ki67 expression on CD8⁺ T cells between dose groups in the tumour.

In comparison, a clear increase in Ki67⁺ expression on CD8⁺ T cells in the blood and spleen was observed at 144 hours post dosing with G1/3H3 in comparison to the IgG1 control. In the blood, whereas the isotype control-treated mice show a mean Ki67⁺ expression on CD8⁺ T cells of 10.4%, the mean expression of Ki67 on CD8⁺ T cells following dosing with G1/3H3 is shown to be 86.3% at 144 hours post dose. In comparison, this increase was not observed with FS22m-063-AA/FS28m-228-010, where the mean Ki67⁺ expression on CD8⁺ T cells was observed at 13.1% following dosing with mAb² in the blood. Similarly, in the spleen, mean Ki67⁺ expression was observed on 36.1% of CD8⁺ T cells following dosing with G1/3H3, in comparison to 8.1% observed following dosing with isotype control and 11.4% observed with FS22m-063-AA/FS28m-228-010.

TABLE 13
The mean percentage of CD8⁺ T cells of total CD3⁺ cells
in the tumour, blood and spleen at 144
hours post dosing with G1-AA/4420, G1/3H3 or
FS22m-063-AA/FS28m-228-010. Data shows mean percentage CD8⁺
T cells of total CD3⁺ T cells ± standard error of the mean.

| | G1-AA/4420 | G1/3H3 | FS22m-063-AA/FS28m-228-010 |
|---|---|---|---|
| | % ± SEM | % ± SEM | % ± SEM |
| Tumour | 32.1 ± 5.8 | 56.1 ± 2.8 | 58.4 ± 5.0 |
| Blood | 22.6 ± 0.6 | 57.0 ± 1.6 | 25.8 ± 0.6 |
| Spleen | 28.8 ± 0.4 | 38.0 ± 0.8 | 29.0 ± 0.9 |

TABLE 14
The mean percentage of Ki67 expressed on CD8⁺ T cells
in the tumour, blood and spleen at 144
hours post dosing with G1-AA/4420, G1/3H3 or
FS22m-063-AA/FS28m-228-010. Data shows mean percentage of Ki67⁺
of total CD8⁺ T cells ± standard error of the mean.

| | G1-AA/4420 | G1/3H3 | FS22m-063-AA/FS28m-228-010 |
|---|---|---|---|
| | % ± SEM | % ± SEM | % ± SEM |
| Tumour | 75.1% ± 2.9 | 85.1% ± 2.8 | 77.6% ± 5.0 |
| Blood | 10.4% ± 1.0 | 86.3% ± 0.7 | 13.1 ± 3.4 |
| Spleen | 8.1% ± 0.3 | 36.1 ± 1.7 | 11.4% ± 1.4 |

Taken together, these data show that the mAb² containing the anti-mouse MLSN mAb FS28m-228-010, FS22m-063-AA/FS28m-228-010, mediates a tumour-specific increase in cytotoxic CD8⁺ T cells in the tumour. Although this is also observed with G1/3H3, this CD137-targeted agonist also promotes a peripheral increase in CD8⁺ T cells in the blood and spleen. Furthermore, these CD8⁺ T cells also show increased proliferation following dosing with G1/3H3.

### REFERENCES

All documents mentioned in this specification are incorporated herein by reference in their entirety.
Altschul S F, Gish W, Miller W, Myers E W, Lipman D J. Basic local alignment search tool. J. Mol. Biol. 215(3), 403-10 (1990).
Altschul S F, Madden T L, SchAffer A A, Zhang J, Zhang Z, Miller W, Lipman D J. Gapped BLAST and PSI-BLAST: a new generation of protein database search programs. Nucleic Acids Res. 25(17), 3389-402 (1997).
Andrade V C, Vettore A L, Felix R S, Almeida M S, Carvalho F, Oliveira J S, Chauffaille M L, Andriolo A, Caballero O L, Zago M A, Colleoni G W. Prognostic impact of cancer/testis antigen expression in advanced stage multiple myeloma patients. Cancer Immun. 8, 2 (2008).
Asgarov K, Balland J, Tirole C, Bouard A, Mougey V, Ramos D, Barroso A, Zangiacomi V, Jary M, Kim S, Gonzalez-Pajuelo M, Royer B, de Haard H, Clark A, Wijdenes J and Borg C (2017). A new anti-mesothelin antibody targets selectively the membrane-associated form. Mabs 9:567-577.
Bagshawe K D, Sharma S K, Springer C J, Antoniw P, Rogers G T, Burke P J, Melton R. Antibody-enzyme conjugates can generate cytotoxic drugs from inactive precursors at tumor sites. Antibody, Immunoconjugates and Radiopharmaceuticals 4, 915-922 (1991).
Bhome R, Bullock M D, Al Saihati H A, Goh R W, Primrose J N, Sayan A E, Mirnezami A H. A top-down view of the tumor microenvironment: structure, cells and signaling. Front. Cel. Dev. Biol. 3, 33 (2015).
Brinkmann U, Kontermann R E. The making of bispecific antibodies. MAbs (9)2, 182-212 (2017) Bruhns P, lannascoli B, England P, Mancardi D A, Fernandez N, Jorieux S, Daëron M. Specificity and affinity of human Fcγ receptors and their polymorphic variants for human IgG subclasses. Blood 113(16), 3716-25 (2009).
Carter P, Smith L, Ryan M. Identification and validation of cell surface antigens for antibody targeting in oncology. Endocr. Relat. Cancer 11(4), 659-87 (2004).
Cheever M A, Allison J P, Ferris A S, Finn O J, Hastings B M, Hecht T T, Mellman I, Prindiville S A, Viner J L, Weiner L M, Matrisian L M. Clin. Cancer Res. 15(17), 5323-37 (2009).
Chen S H, Hung W C, Wang P, Paul C, Konstantopoulos K (2013). Mesothelin binding to CA125/MUC16 promotes pancreatic cancer cell motility and invasion via MMP-7 activation. Sci Rep. 3: 1870.
Chen D S, Mellman I. Oncology meets immunology: the cancer-immunity cycle. Immunity 39(1), 1-10 (2013).
Creaney J, Dick I M and Robinson B W (2015). Discovery of new biomarkers for malignant mesothelioma. Curr Pulmonol Rep. 4: 15-21.
Cui A, Jin X0G, Zhai K, Tong Z-H and Shi H-Z (2014). Diagnostic values of soluble mesothelin-related peptides for malignant pleural mesothelioma: updated meta-analysis. BMJ Open 4: e004145.
Grisshammer, R. and Nagai, K. (1995) Purification of overproduced proteins from E. coli cells In: DNA Cloning 2: Expression systems (Rickwood, D. and Hames, B. D., Eds.), The Practical Approach Series, pp. 59-92. IRL Press, Oxford University Press.
Gubin M M, Artyomov M N, Mardis E R, Schreiber R D. Tumor neoantigens: building a framework for personalized cancer immunotherapy. J. Clin. Invest. 125(9), 3413-21 (2015).
Gure A O, Chua R, Williamson B, Gonen M, Ferrera C A, Gnjatic S, Ritter G, Simpson A J, Chen Y T, Old L J, Altorki N K. Cancer-testis genes are coordinately expressed and are markers of poor outcome in non-small cell lung cancer. Clin. Cancer Res. 11(22), 8055-62 (2005).
Hassan R, Kindler H L, Jahan T, Bazhenova L, Reck M, Thomas A, Pastan I, Parno J, O'Shannessy D J, Fatato P, Maltzman J D and Wallin B A (2014). Phase II clinical trial of amatuximab, a chimeric antimesothelin antibody with pemetrexed and cisplatin in advanced unresectable pleural mesothelioma. Clin. Canc. Res. 20:5927-5936.
Hassan R, Kreitman R J, Pastan I and Willingham M C (2005). Localization of mesothelin in epithelial ovarian cancer. Appl Immunohistochem Mol Morphol AIMM Off Publ Soc Appl Immunohistochem 13:243-47;
Hassan R, Lerner M R, Benbrook D, Lightfoot S A, Brackett D J, Wang Q C and Pastan I (2002). Clin. Cancer Res.: 8: 3520-3526.
Hassan R, Thomas, A, Alewine, C, Le, DT, Jaffee, EM and Pastan I (2016). Mesothelin immunotherapy for cancer: ready for prime time?J. Clin. Onc. 34:4171-4180.
Hezareh M, Hessell A J, Jensen R C, van de Winkel J G and Parren P W. Effector function activities of a panel of mutants of a broadly neutralizing antibody against human immunodeficiency virus type 1. J. Virol. 75(24), 12161-8 (2001).
Hezareh M, Hessell A J, Jensen R C, van de Winkel J G, Parren P W. Effector function activities of a panel of mutants of a broadly neutralizing antibody against human immunodeficiency virus type 1. J. Virol. 75(24), 12161-8 (2001).
Hollevoet K, Reitsma J B, Creaney J, Grigoriu, BD, Robinson B W, Scherpereel A, Cristaudo A, Pass H I, Nackaerts K, Rodriques Portal J A, Schneider J, Muley, T, Di Serio F, Baas P, Tomasetti M, Rai A J and van Meerbeeck J P. Serum mesothelin for diagnosing malignant pleural mesothelioma: an individual patient data meta-analysis. J Clin. Oncol. 30 (13), 1541-1549 (2012).
Holliger P, Hudson P J. Engineered antibody fragments and the rise of single domains. Nat. Biotechnol. 23(9), 1126-36 (2005).
Idusogie E E, Presta L G, Gazzano-Santoro H, Totpal K, Wong P Y, Ultsch M, Meng Y G, Mulkerrin M G. Mapping of the Clq binding site on rituxan, a chimeric antibody with a human IgG1 Fc. J. Immunol. 164(8), 4178-84 (2000).
Kabat E A, Wu T T, Perry H M, Gottesman K S, Foeller C. Sequences of Proteins of Immunological Interest, 5th ed. NIH Publication No. 91-3242. Washington, D.C.: U.S. Department of Health and Human Services (1991).
Kaneko O, Gong, L, Zhang, J, Hansen, JK, Hassan, R, Lee B and Ho M (2009). A binding domain on mesothelin for CA125/MUC16. J. Biol. Chem. 284: 3739-3749.
Klein C, Schaefer W, Regula J T. The use of CrossMAb technology for the generation of bi- and multispecific antibodies. MAbs 8(6), 1010-20 (2016).
Kontermann (2012). Dual targeting strategies with bispecific antibodies. MAbs 4(2):182-97.
Ledermann J A, Begent R H, Massof C, Kelly A M, Adam T, Bagshawe K_{D}. A phase-I study of repeated therapy with radiolabelled antibody to carcinoembryonic antigen using intermittent or continuous administration of cyclosporin A to supress the immune response. Int. J. Cancer 47(5), 659-64 (1991).
Lee J-H, Kim H, Yao Z, Szajek L, Grasso L, Kim I and Paik C. H (2018). Tumour-Shed Antigen Affects Antibody Tumour Targeting: Comparison of Two ⁸⁹Zr-Labeled Antibodies Directed against Shed or Nonshed Antigens”. Contrast Media and Molecular Imaging. ID: 2461257.
Lefranc M P, Giudicelli V, Duroux P, Jabado-Michaloud J, Folch G, Aouinti S, Carillon E, Duvergey H, Houles A, Paysan-Lafosse T, Hadi-Saljoqi S, Sasorith S, Lefranc G, Kossida S. IMGT®, the international ImMunoGeneTics information system@25 years on. Nucleic Acids Res. 43 (Database issue), D413-22 (2015).
Ma J, Tang W K, Esser L, Pastan I, Xia D. (2012). Recognition of mesothelin by the therapeutic antibody MORAb-009: structural and mechanistic insights. J Biol. Chem. 287: 33123-31.
Malarkannan S, Horng T, Shih P P, Schwab S, Shastri N. Presentation of out-of-frame peptide/MHC class I complexes by a novel translation initiation mechanism. Immunity 10(6), 681-90 (1999).
Napoletano C, Bellati F, Tarquini E, Tomao F, Taurino F, Spagnoli G, Rughetti A, Muzii L, Nuti M, Benedetti Panici P. MAGE-A and NY-ESO-1 expression in cervical cancer: prognostic factors and effects of chemotherapy. Am. J. Obstet. Gynecol. 198(1), 99.e1-99.e7 (2008).
Pearson W R, Lipman D J. Improved tools for biological sequence comparison. Proc. Natl. Acad. Sci. U.S.A. 85(8), 2444-8 (1988).
Podojil J R, Miller S D. Potential targeting of B7-H4 for the treatment of cancer. Immunol. Rev. 276(1), 40-51 (2017).
Powers G A, Hudson P J, Wheatcroft M P. Design and production of multimeric antibody fragments, focused on diabodies with enhanced clinical efficacy. Methods Mol. Biol. 907, 699-712 (2012)
Rickert K W, Grinberg L, Woods R M, Wilson S, Bowen M A, Baca M. Combining phage display with de novo protein sequencing for reverse engineering of monoclonal antibodies. MAbs, 2016; 8(3):501-12
Rosenberg S. Development of Cancer Vaccines. ASCO Educational Book Spring: 60-62 (2000).
Sapede, C, Gauvrit A, Barbieux I, Padieu M, Cellerin L, Sagan C, Scherpereel A, Dabouis G, Gregoire M. Abberant splicing and protease involvement in mesothelin release from epithelioid mesothelioma cells. Canc. Sci 99(3): 590-594 (2008)
Scott A M, Renner C. Tumour Antigens Recognized by Antibodies. eLS (2001).
Simpson A J, Cabellero O L, Jungbluth O L, Chen Y T, Old L J. Cancer/testis antigens, gametogenesis and cancer. Nat. Rev. Cancer 5(8), 615-25 (2005).
Smith T F, Waterman M S. Identification of common molecular subsequences. J. Mol. Biol. 147(1), 195-7 (1981).
Spiess, Zhai, Carter (2015). Alternative molecular formats and therapeutic applications for bispecific antibodies. Molecular Immunology. 67:95-106.
Tai Y T, Anderson K C. Targeting B-cell maturation antigen in multiple myeloma. Immunotherapy 7(11), 1187-99 (2015).
Tang Z, Feng, M, Gao, W, Chen W, Chaudhary, A, Croix, BS, Qian M, Dimitrov D S and Ho M, (2013). A human single-domain antibody elicits potent antitumor activity by targeting an epitope in mesothelin close to the cancer cell surface. Mol. Canc. Ther. 12:416-426.
Tinguely M, Jenni B, Knights A, Lopes B, Korol D, Rousson V, Curioni Fontecedro A, Cogliatti Bittermann A G, Schmid U, Dommann-Scherrer C, Maurer R, Renner C, Probst-Hensch N M, Moch H, Knuth A, Zippelius A. MAGE-C1/CT-7 expression in plasma cell myeloma: sub-cellular localization impacts on clinical outcome. Cancer Sci. 99(4), 720-5 (2008).
Tomasetti M, Rai A J and van Meerbeeck J P (2012). Serum mesothelin for diagnosing malignant pleural mesothelioma: an individual patient data meta-analysis. J Clin. Oncol. 30:1541-1549.
Velazquez E F, Jungbluth A A, Yancovitz M, Gnjatic S, Adams S, O'Neill D, Zavilevich K, Albukh T, Christos P, Mazumdar M, Pavlick A, Polsky D, Shapiro R, Berman R, Spira J, Busam K, Osman I, Bhardwaj N. Expression of the cancer/testis antigen NY-ESO-1 in primary and metastatic malignant melanoma (MM)—correlation with prognostic factors. Cancer Immun. 7, 11 (2007).
Wang X, Mathieu M, Brezski R J. IgG Fc engineering to modulate antibody effector functions. Protein Cell 9(1), 63-73 (2018).
Wozniak-Knopp G, Bartl S, Bauer A, Mostageer M, Woisetschläger M, Antes B, Ettl K, Kainer M, Weberhofer G, Wiederkum S, Himmler G, Mudde G C, Rüker F. Introducing antigen-binding sites in structural loops of immunoglobulin constant domains: Fc fragments with engineered HER2/neu-binding sites and antibody properties. Protein Eng. Des. Sel. 23(4), 289-97 (2010).
Zhang, Y, Chertov, O, Zhang, J, Hassan, R and Pastan I (2011). Cytotoxic activity of immunotoxin SS1P is modulated by TACE-dependent mesothelin shedding. Cancer Res. 71: 5915-5922.
Zhao X Y, Subramanyam B, Sarapa N, Golfier S and Dinter H (2016). Novel antibody therapeutics targeting mesothelin in solid tumours. Clin. Cancer Drugs 3:76-86.

1. An antibody molecule that binds mesothelin (MSLN), wherein the antigen-binding site of the antibody molecule comprises the heavy chain variable domain (VH) CDR1, VH CDR2, VH CDR3, light chain variable domain (VL) CDR1, VL CDR2 and VL CDR3 set forth in:
(i) SEQ ID NO: 98, 73, 99, 20, 21, and 44, respectively;
(ii) SEQ ID NO: 10, 11, 41, 20, 21 and 22, respectively;
(iii) SEQ ID NO: 98, 73, 99, 20, 21 and 34, respectively;
(iv) SEQ ID NO: 98, 73, 99, 20, 21 and 25, respectively;
(v) SEQ ID NO: 101, 73, 103, 20, 21 and 34, respectively;
(vi) SEQ ID NO: 98, 73, 99, 20, 21 and 43, respectively;
(vii) SEQ ID NO: 101, 73, 103, 20, 21 and 43, respectively;
(viii) SEQ ID NO: 98, 73, 99, 20, 21 and 44, respectively;
(ix) SEQ ID NO: 85, 73, 75, 20, 21 and 34, respectively;
(x) SEQ ID NO: 85, 73, 75, 20, 21 and 43, respectively;
(xi) SEQ ID NO: 111, 73, 113, 20, 21 and 25, respectively;
(xii) SEQ ID NO: 101, 73, 103, 20, 21 and 25, respectively;
(xiii) SEQ ID NO: 71, 73, 75, 20, 21 and 25, respectively;
(xiv) SEQ ID NO: 10, 11, 32, 20, 21 and 22, respectively;
(xv) SEQ ID NO: 10, 11, 51, 20, 21 and 22, respectively; or
(xvi) SEQ ID NO: 10, 11, 12, 20, 21 and 22, respectively; and
wherein the CDR sequences are defined according to the ImMunoGeneTics (IMGT) numbering scheme; and/or
wherein the antigen-binding site of the antibody molecule comprises the VH CDR1, VH CDR2, VH CDR3, VL CDR1, VL CDR2 and VL CDR3 set forth in:
(i) SEQ ID NO: 97, 182, 100, 23, 24 and 44, respectively;
(ii) SEQ ID NO: 13, 14, 42, 23, 24 and 22, respectively;
(iii) SEQ ID NO: 97, 74, 100, 23, 24 and 34, respectively;
(iv) SEQ ID NO: 97, 74, 100, 23, 24 and 25, respectively;
(v) SEQ ID NO: 102, 74, 104, 23, 24 and 34, respectively;
(vi) SEQ ID NO: 97, 74, 100, 23, 24 and 43, respectively;
(vii) SEQ ID NO: 102, 74, 104, 23, 24 and 43, respectively;
(viii) SEQ ID NO: 97, 74, 100, 23, 24 and 44, respectively;
(ix) SEQ ID NO: 86, 74, 76, 23, 24 and 34, respectively;
(x) SEQ ID NO: 86, 74, 76, 23, 24 and 43, respectively;
(xi) SEQ ID NO: 112, 74, 114, 23, 24 and 25, respectively;
(xii) SEQ ID NO: 102, 74, 104, 23, 24 and 25, respectively;
(xiii) SEQ ID NO: 72, 74, 76, 23, 24 and 25, respectively;
(xiv) SEQ ID NO: 13, 14, 33, 23, 24 and 22, respectively;
(xv) SEQ ID NO: 13, 14, 52, 23, 24 and 22, respectively;
(xvi) SEQ ID NO: 13, 14, 15, 23, 24 and 22, respectively; and
wherein the CDR sequences are defined according to the Kabat numbering scheme.

2. The antibody molecule according to claim 1, comprising the heavy chain variable (VH) domain and the light chain variable (VL) domain set forth in:
(i) FS28-256-271 set forth in SEQ ID NO: 180, and 56, respectively;
(ii) FS28-024-052 set forth in SEQ ID NO: 39 and 18, respectively;
(iii) FS28-256-021 set forth in SEQ ID NO: 109 and 93, respectively;
(iv) FS28-256-012 set forth in SEQ ID NO: 109 and 79, respectively;
(v) FS28-256-023 set forth in SEQ ID NO: 121 and 93, respectively;
(vi) FS28-256-024 set forth in SEQ ID NO: 109 and 53, respectively;
(vii) FS28-256-026 set forth in SEQ ID NO: 121 and 53, respectively;
(viii) FS28-256-027 set forth in SEQ ID NO: 109 and 56, respectively;
(ix) FS28-256-001 set forth in SEQ ID NO: 63 and 93, respectively;
(x) FS28-256-005 set forth in SEQ ID NO: 63 and 53, respectively;
(xi) FS28-256-014 set forth in SEQ ID NO: 115 and 79, respectively;
(xii) FS28-256-018 set forth in SEQ ID NO: 121 and 79, respectively;
(xiii) FS28-256 set forth in SEQ ID NO: 69 and 79, respectively;
(xiv) FS28-024-051 set forth in SEQ ID NO: 30 and 18, respectively;
(xv) FS28-024-053 set forth in SEQ ID NO: 49 and 18, respectively; or
(xvi) FS28-024 set forth in SEQ ID NO: 8 and 18, respectively.

3. The antibody molecule according to claim 1, wherein the antibody molecule comprises the VH CDR1, VH CDR2, VH CDR3, VL CDR1, VL CDR2 and VL CDR3 set forth in SEQ ID NO: 98, 73, 99, 20, 21 and 44, respectively, wherein the CDR sequences are defined according to the IMGT numbering scheme; the VH CDR1, VH CDR2, VH CDR3, VL CDR1, VL CDR2 and VL CDR3 set forth in SEQ ID NO: 97, 182, 100, 23, 24 and 44, respectively, wherein the CDR sequences are defined according to the Kabat numbering scheme; and/or the VH domain and the VL domain set forth in SEQ ID NO: 180 and 56, respectively.

4. The antibody molecule according to claim 1, wherein the antibody molecule comprises the VH CDR1, VH CDR2, VH CDR3, VL CDR1, VL CDR2 and VL CDR3 set forth in SEQ ID NO: 10, 11, 41, 20, 21 and 22, respectively, wherein the CDR sequences are defined according to the IMGT numbering scheme; the VH CDR1, VH CDR2, VH CDR3, VL CDR1, VL CDR2 and VL CDR3 set forth in SEQ ID NO: 13, 14, 42, 23, 24 and 22, respectively, wherein the CDR sequences are defined according to the Kabat numbering scheme; and/or the VH domain and the VL domain set forth in SEQ ID NO: 39 and 18, respectively.

5. The antibody molecule according to claim 1, wherein the antibody molecule is a multispecific antibody molecule and comprises a second antigen-binding site that binds a second antigen.

6. The antibody molecule according to claim 5, wherein the second antigen-binding site is located in a constant domain of the antibody molecule.

7. The antibody molecule according to claim 6, wherein the constant domain is a CH3 domain.

8. The antibody molecule according to claim 5, wherein the second antigen-binding site binds an immune cell antigen.

9. The antibody molecule according to claim 8, wherein the immune cell antigen is a member of the tumour necrosis factor receptor superfamily (TNFRSF).

10. The antibody molecule according to claim 9, wherein the member of the TNFRSF is CD137.

11. The antibody molecule according to claim 6, wherein the second antigen-binding site comprises a first sequence, a second sequence, and/or a third sequence, wherein the first sequence, the second sequence and the third sequence are located in the AB structural loop, the CD structural loop and the EF structural loop of the constant domain, respectively.

12. The antibody molecule according to claim 8, wherein the antibody molecule is capable of activating an immune cell in the presence of MSLN.

13. The antibody molecule according to claim 12, wherein the immune cell is a T cell, B cell, natural killer (NK) cell, natural killer T (NKT) cell, or dendritic cell (DC).

14. The antibody molecule according to claim 1, wherein the antibody molecule has been modified to reduce or abrogate binding of the CH2 domain of the antibody molecule to one or more Fcγ receptors.

15. The antibody molecule according to claim 14, wherein the antibody molecule does not bind to one or more Fcγ receptors.

16. (canceled)

17. A nucleic acid molecule or molecules encoding the antibody molecule according to claim 1.

18. (canceled)

19. A recombinant host cell comprising the nucleic acid molecule(s) according to claim 17.

20. (canceled)

21. A pharmaceutical composition comprising the antibody molecule according to claim 1 and a pharmaceutically acceptable excipient.

22. (canceled)

23. A method of detecting or diagnosing a cancer in an individual, the method comprising the use of the antibody molecule according to claim 1.

24. A method of treating cancer in an individual comprising administering to the individual a therapeutically effective amount of the antibody molecule according to claim 1.
